(19) United States
(12) Patent Application Publication
(10) Pub. No.: US 2023/0114305 A1
(43) Pub. Date: Apr. 13, 2023

(54) RECOMBINANT MYXOMA VIRUSES AND USES THEREOF

(71) Applicants: Eric Bartee, John's Island, SC (US); Ramon MORENO, Scottsdale, AZ (US); Spyro MOUSSES, Scottsdale, AZ (US)

(72) Inventors: Eric Bartee, John's Island, SC (US); Ramon MORENO, Scottsdale, AZ (US); Spyro MOUSSES, Scottsdale, AZ (US)

(21) Appl. No.: 17/801,206

(22) PCT Filed: Feb. 19, 2021
(86) PCT No.: PCT/US2021/018686

§ 371 Date: Aug. 19, 2022

Related U.S. Application Data
(60) Provisional application No. 62/979,232, filed on Feb. 20, 2020.

(51) Int. Cl. A61K 35/768; C12N 15/86

(52) U.S. Cl. CPC A61K 35/768; C12N 15/86; A61K 2039/5256

(57) ABSTRACT

The present disclosure provides a recombinant oncolytic myxoma vims engineered to express a soluble form of an immune checkpoint protein in conjunction with a cytokine/chemokine and/or a tumor antigen. In certain aspects, the oncolytic myxoma virus is a replication competent virus such as myxoma vims. Methods of cancer treatment comprising administering the recombinant oncolytic myxoma virus expressing the soluble form of the immune checkpoint protein are also provided.

### PRIORITY CLAIM

This application claims benefit of priority to U.S. Provisional Application Ser. No. 62/979,232, filed Feb. 20, 2020, the entire contents of which is hereby incorporated by reference.

### BACKGROUND

### 1. Field

The present disclosure relates generally to the field of molecular biology and medicine. More particularly, it concerns oncolytic viruses expressing the immune checkpoint protein PD1 and cytokines/chemokines and/or tumor antigens.

### 2. Description of Related Art

Current treatments used to treat various types of cancer tend to work by poisoning or killing the cancerous cell. Unfortunately, treatments that are toxic to cancer cells typically tend to be toxic to healthy cells as well. Moreover, the heterogeneous nature of tumors is one of the primary reasons that effective treatments for cancer remain elusive. Current mainstream therapies such as chemotherapy and radiotherapy tend to be used within a narrow therapeutic window of toxicity. These types of therapies have limited applicability due to the varying types of tumor cells and the limited window in which these treatments can be administered. Modern anticancer therapies currently being developed attempt to selectively target tumor cells while being less toxic to healthy cells, thereby being more likely to leave healthy cells unaffected.

Metastatic melanoma is an aggressive disease with a 16% 5-year survival rate and responds poorly to most standard chemotherapies. Interferon and interleukin 2 (IL-2) have both been approved by the U.S. Food and Drug Administration for the treatment of melanoma. Both mediate their benefit by stimulating an antitumor immune response. However, toxicity and low response rates have limited their use significantly. The first immune-checkpoint inhibitor approved by the U.S. Food and Drug Administration (FDA) was ipilimumab, a fully human immunoglobulin G1 monoclonal antibody that blocks cytotoxic T-lymphocyte antigen (CTLA)-4 and consequently the PD-1 pathway for the treatment of metastatic melanoma in 2011. The finding that programmed cell death protein 1 ligand 1 (PDL1 or PD-L1) and PDL2 are expressed by melanoma cells, T cells, B cells and natural killer cells led to the development of programmed cell death protein 1 (PD1 or PD-1)-specific antibodies (e.g., nivolumab and pembrolizumab).

Thus, PD1 pathway blockade has become a major focus in anticancer drug development beyond melanoma. In addition to benefiting patients with renal cell carcinoma, it has reported benefit in patients with tumors previously not considered sensitive to immunotherapies, including non-small cell lung cancer. However, there are still limitations due to toxicity associated with these immunotherapies. Thus, there is a need for an immunotherapy blocking the PD1 pathway with the best balance of high efficacy and low toxicity.

Immune checkpoint inhibition in cancer therapy has been shown to be effective for the treatment of a number of different types of cancer. However, not all cancers cells respond equally. Additionally, toxicity and the development of resistance to individual checkpoint inhibitors are problematic (Pardoll, 2012; Topalian et al., 2015). Improvements for immune checkpoint inhibitors are needed to combat aforementioned drawbacks.

Another promising therapeutic approach for cancer therapy is the use of oncolytic viruses. Treatment with oncolytic viruses by themselves and combined with other therapies elicit direct tumor cytotoxicity and potentiate activation of immune cells against tumor cells. Oncolytic viruses possess novelty in that they can also be ‘armed’ to express proteins to make them more effective (Kaufman et al., 2015).

Recent work has experimentally shown the efficacy of combining oncolytic virus with immune checkpoint inhibitor by engineering a myxoma virus to express a human extracellular portion of the PD1 checkpoint molecule (Bartee et al., 2017). PD1 is a membrane protein on T-cells that binds to PDL1 on tumor cells. This interaction triggers signaling through PD1 leading to inhibition of activation of T-cells toward tumor cells, thus protecting tumor cells from immune cell elimination (Pardoll, 2012). Upon infection of tumor cells, through direct injection in the tumor, of the myxoma virus expressing the extracellular PD1 protein (vPD1), the interaction of PDL1 on tumor and PD1 on T-cells in inhibited locally. This occurs by the extracellular PD1 protein directly binding to tumor cell PDL1 blocking T-cell PD1 from binding PDL1, leading to T-cell immune activation and anti-tumor effect. There is an unmet need for improved methods of inhibiting immune checkpoints.

### SUMMARY

Certain embodiments of the present disclosure provide a recombinant oncolytic myxoma virus comprising expression cassettes encoding (a) a soluble form of programmed cell death protein 1 (PD1); (b) interleukin 12 (IL-12); and (c) a tumor antigen or a cytokine/chemokine other than interleukin 12 (IL-12), wherein the myxoma virus is replication competent and wherein two of said expression cassettes are provided a dicistronic expression cassette.

In some aspects, the virus comprises a cytokine/chemokine selected from the group consisting of IL-2, IL-4, IL-15, IL-17, IL-18 (mutated), IL-23, IL-35, IL-36, IFN-γ, IFN-β, RANTES/CCL5, GM-CSF, cGAS, or Ebola GP (aa1-298). In particular aspects, the virus comprises a tumor antigen selected from the group consisting of p53, MUC1, PSMA, mRAS or S100P.

In certain aspects, the soluble PD1/mutant soluble PD1 comprises an extracellular region of human PD1. In some aspects, the soluble PD1/mutant soluble PD1 and the IL-12 are encoded in the dicistronic expression cassette. For example, the soluble PD1/mutant soluble PD1 and the IL-12 may be encoded in distinct expression cassettes. In some aspects, the soluble PD1/mutant soluble PD1 and the chemokine/cytokine are encoded in the dicistronic expression cassette. In certain aspects, the soluble PD1/mutant soluble PD1 and the chemokine/cytokine are encoded in distinct expression cassettes. In certain aspects, the soluble PD1/mutant soluble PD1 and the tumor antigen are encoded in the dicistronic expression cassette. In particular aspects, the soluble PD1/mutant soluble PD1 and the tumor antigen are encoded in distinct expression cassettes. In some aspects, the IL-12 and the tumor antigen are encoded in the dicistronic expression cassette. In specific aspects, the IL-12 and the tumor antigen are encoded in distinct expression cassettes.

In some aspects, a first expression cassette is inserted in the intergenic region between the m135r and m136r ORFs and a second expression cassette is inserted between the m152r and m154r ORFs and replaces the mR153r ORF.

In certain aspects, the dicistronic expression cassette comprises an internal ribosome entry site (IRES) between the coding sequences of the expression cassettes. For example, the IRES is a cellular IRES, such as an IRES from eIF4G, BCL2, BiP, or c-IAP1. In certain aspects, the IRES is a viral IRES. In some aspects, the IRES is an IRES from poliovirus (PV), encephalomyelocarditis virus (EMCV), classical swine-fever virus (CSFV), foot-and-mouth disease virus (FMDV), human immunodeficiency virus (HIV), bovine viral diarrhea virus (BVDV), hepatitis C virus (HCV) or cricket paralysis virus (CrPV). In particular aspects, the IRES is an IRES from HCV.

In some aspects, the dicistronic expression cassette comprises a polyprotein of the coding sequences of the expression cassettes. In certain aspects, the polyprotein comprises a protease cleavage site between the proteins encoded by the two expression cassettes. The protease cleavage site may be cleaved by cellular protease. For example, the protease cleavage site is a self-cleaving peptide, such as a viral self-cleaving peptide, such as a T2A, P2A, E2A or F2A peptide.

In certain aspects, the expression cassette(s) is/are under the control of one or more viral promoters. In some aspects, the one or more viral promoters is/are synthetic early/late poxvirus promoter. For example, the synthetic early/late poxvirus promoter is at least 90% identical to SEQ ID NO: 14.

In additional aspects, the virus further comprises a marker gene. In some aspects, IL-12 is fused to a transmembrane domain. For example, the transmembrane domain is encoded by SEQ ID NO: 12. In particular aspects, the oncolytic myxoma virus is encoded by SEQ ID NO: 13.

A further embodiment provides a recombinant myxoma oncolytic virus comprising one or more expression cassettes encoding a (a) mutant soluble form of PD1 (mutPD1), (b) interleukin 12 (IL-12), and (c) a cytokine/chemokine other than interleukin 12 (IL-12) or a tumor antigen, wherein the myxoma virus is replication competent, and wherein the mutPD1 prevents recognition of mutPD1 by an anti-PD1 antibody.

In certain aspects, the virus comprises a cytokine/chemokine selected from the group consisting of IL-2, IL-4, IL-15, IL-17, IL-18 (mutated), IL-23, IL-35, IL-36, IFN-γ, IFN-β, RANTES/CCL5, GM-CSF, cGAS, or Ebola GP (aa1-298). In some aspects, the virus comprises a tumor antigen selected from the group consisting of p53, MUC1, PSMA, mRAS or S100P.

In particular aspects, the mutPD1 contains a mutation in the CD loop that prevents antibody recognition by anti-PD1 antibodies. In specific aspects, the mutPD1 contains a point mutation in the CD loop comprising D85G. In some aspects, the mutPD1 is not recognized by pembrolizumab.

In certain aspects, the soluble PD1/mutant soluble PD1 comprises an extracellular region of human PD1. In some aspects, the soluble PD1/mutant soluble PD1 and the IL-12 are encoded in the dicistronic expression cassette. For example, the soluble PD1/mutant soluble PD1 and the IL-12 may be encoded in distinct expression cassettes. In some aspects, the soluble PD1/mutant soluble PD1 and the chemokine/cytokine are encoded in the dicistronic expression cassette. In certain aspects, the soluble PD1/mutant soluble PD1 and the chemokine/cytokine are encoded in distinct expression cassettes. In certain aspects, the soluble PD1/mutant soluble PD1 and the tumor antigen are encoded in the dicistronic expression cassette. In particular aspects, the soluble PD1/mutant soluble PD1 and the tumor antigen are encoded in distinct expression cassettes. In some aspects, the IL-12 and the tumor antigen are encoded in the dicistronic expression cassette. In specific aspects, the IL-12 and the tumor antigen are encoded in distinct expression cassettes.

In some aspects, a first expression cassette is inserted in the intergenic region between the m135r and m136r ORFs and a second expression cassette is inserted between the m152r and m154r ORFs and replaces the mR153r ORF.

In certain aspects, the dicistronic expression cassette comprises an internal ribosome entry site (IRES) between the coding sequences of the expression cassettes. For example, the IRES is a cellular IRES, such as an IRES from eIF4G, BCL2, BiP, or c-IAP1. In certain aspects, the IRES is a viral IRES. In some aspects, the IRES is an IRES from poliovirus (PV), encephalomyelocarditis virus (EMCV), classical swine-fever virus (CSFV), foot-and-mouth disease virus (FMDV), human immunodeficiency virus (HIV), bovine viral diarrhea virus (BVDV), hepatitis C virus (HCV) or cricket paralysis virus (CrPV). In particular aspects, the IRES is an IRES from HCV.

In some aspects, the dicistronic expression cassette comprises a polyprotein of the coding sequences of the expression cassettes. In certain aspects, the polyprotein comprises a protease cleavage site between the proteins encoded by the two expression cassettes. The protease cleavage site may be cleaved by cellular protease. For example, the protease cleavage site is a self-cleaving peptide, such as a viral self-cleaving peptide, such as a T2A, P2A, E2A or F2A peptide.

In certain aspects, the expression cassette(s) is/are under the control of one or more viral promoters. In some aspects, the one or more viral promoters is/are synthetic early/late poxvirus promoter. For example, the synthetic early/late poxvirus promoter is at least 85%, 90% or 95% identical to AAAATTGAAATTTTATTTTTTTTTTTTGGAATATAAATA (SEQ ID NO: 14).

In additional aspects, the virus further comprises a marker gene. In some aspects, IL-12 is fused to a transmembrane domain. For example, the transmembrane domain is encoded by SEQ ID NO: 12. In particular aspects, the oncolytic myxoma virus comprises an expression construct selected from those of FIGS. 22-24.

Further provided herein is a pharmaceutical composition of the oncolytic myxoma virus of the present embodiments and aspects thereof.

Another embodiment provides a method of treating a disease in a subject in need thereof comprising administering an effective amount of the oncolytic myxoma virus of the present embodiment or aspects thereof.

In some aspects, the disease is cancer. The cancer may have increased expression of programmed death-ligand 1 (PDL1). In certain aspects, the subject has been determined to have a cancer that expresses increased PDL1. In other aspects, the cancer does not have increased expression of PDL1. In particular aspects, the cancer is melanoma (e.g., metastatic melanoma), kidney cancer, colorectal cancer, breast cancer, lung cancer, head and neck cancer, brain cancer, leukemia, prostate cancer, bladder cancer, and ovarian cancer.

In certain aspects, the oncolytic myxoma virus is administered intra-arterially, intravenously, intraperitoneally, or intratumorally. In some aspects, the oncolytic myxoma virus is administered two or more times.

In additional aspects, the method further comprises administering at least a second anti-cancer therapy to the subject. In some aspects, the second anti-cancer therapy is administered concurrently or sequentially with the recombinant virus. In particular aspects, the second anti-cancer therapy is an immunomodulator. In some aspects, the second anti-cancer therapy is immunotherapy, chemotherapy, radiotherapy, gene therapy, surgery, hormonal therapy, anti-angiogenic therapy or cytokine therapy. In certain aspects, the immunotherapy is immune checkpoint inhibitor therapy. In specific aspects, the immune checkpoint inhibitor therapy comprises treatment with an antibody directed to PD1, PDL1, or CTLA4. For example, the antibody is Pembrolizumab, Nivolumab, Atezolizumab, Avelumab, Durvalumab, or Ipilimumab.

In yet another embodiment, there is provided a method of treating a disease in a subject in need thereof comprising (a) testing the subject for overexpression of PDL1; and (b) administering to a subject with increased expression of PDL1 a therapeutically effective amount of the oncolytic myxoma virus of claim 1 or 4.

In some aspects, the disease is cancer. The cancer may have increased expression of programmed death-ligand 1 (PDL1). In certain aspects, the subject has been determined to have a cancer that expresses increased PDL1. In other aspects, the cancer does not have increased expression of PDL1. In particular aspects, the cancer is melanoma (e.g., metastatic melanoma), kidney cancer, colorectal cancer, breast cancer, lung cancer, head and neck cancer, brain cancer, leukemia, prostate cancer, bladder cancer, and ovarian cancer.

In certain aspects, the oncolytic myxoma virus is administered intra-arterially, intravenously, intraperitoneally, or intratumorally. In some aspects, the oncolytic myxoma virus is administered two or more times.

In additional aspects, the method further comprises administering at least a second anti-cancer therapy to the subject. In some aspects, the second anti-cancer therapy is administered concurrently or sequentially with the recombinant virus. In particular aspects, the second anti-cancer therapy is an immunomodulator. In some aspects, the second anti-cancer therapy is immunotherapy, chemotherapy, radiotherapy, gene therapy, surgery, hormonal therapy, anti-angiogenic therapy or cytokine therapy. In certain aspects, the immunotherapy is immune checkpoint inhibitor therapy. In specific aspects, the immune checkpoint inhibitor therapy comprises treatment with an antibody directed to PD1, PDL1, or CTLA4. For example, the antibody is Pembrolizumab, Nivolumab, Atezolizumab, Avelumab, Durvalumab, or Ipilimumab.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1—Schematics of recombinant viral genomic structures.

FIG. 2—vPD1-IL2 efficacy study in subcutaneous B16F10 (B16F10 PD1L-KO) contralateral xenograft model.

FIG. 3—vPD1-IL12 efficacy study in subcutaneous B16F10 (B16F10 PD1L-KO) contralateral xenograft model.

FIG. 4—vPD1-IL15 efficacy study in subcutaneous B16F10 (B16F10 PD1L-KO) contralateral xenograft model.

FIG. 5—vPD1-IL18 efficacy study in subcutaneous B16F10 (B16F10 PD1L-KO) contralateral xenograft model.

FIG. 6—In vivo SC contralateral model. Starting tumor size.

FIG. 7—In vivo SC contralateral model. Treatment results are shown. The studies demonstrate that vPD1/IL12 constructs were superior to other constructs tested.

FIG. 8—Sequence alignment of the C′D loop in ectodomains of PD-1. Secondary structural elements of human PD-1 (hPD-1) are shown on top of the alignment while those of murine PD-1 (mPD-1) are shown at the bottom. (SEQ ID NOS: 19-23)

FIG. 9—Schematic depicting therapy with soluble TIM3 myxoma virus.

FIG. 10: MYXV therapy induces TIM3 on CD8+ T cells and NK cells. Analysis of TIM3 expression on the indicated immunological subsets six days after initiation of viral treatment.

FIGS. 11A-11C—TIM3 blockade improves MYXV treatment of melanoma. SQ B16/F10 tumors were treated as indicated. (FIG. 11A) Tumor volume as a percent of starting volume. Complete eradication of visible tumor is marked with white circles. (FIG. 11B) Overall survival of animals. (FIG. 11C) Example of alopecia observed in animals.

FIGS. 12A-12D—vTIM3 secretes soluble TIM3 from infected cells. (FIG. 12A) Schematic of the genomic structure of vGFP and vTIM3. (FIG. 12B) Production of new virus in B16/F10 cells. (FIG. 12C) MTT assay analyzing cellular viability 24 hours post infection. (FIG. 12D) Expression of TIM3 transgene.

FIGS. 13A-13C—vTIM3 duplicates efficacy of combination therapy with reduced toxicities. (FIG. 13A) Tumor volume as a percent of starting volume. Complete eradication of visible tumor is marked with white circles. (FIG. 13B) Overall survival of animals (FIG. 13C) Average alopecia score observed in animals treated with the indicated therapy.

FIGS. 14A-14B—Generation of mutations in TIM3. (FIG. 14A) Schematic of proposed mutations for TIM3 transgenes. (SEQ ID NO: 24) (FIG. 14B) Expression of mutated TIM3 out of newly generated recombinant vTIM3 mutant viruses. Note that the GALS mutant runs at a lower MW due to the loss of glycosylation.

FIGS. 15A-15C—(FIG. 15A) Schematic depicting mouse study. (FIG. 15B) Individual tumor growth over time. (FIG. 15C) Overall survival.

FIGS. 16A-B—vPD1 is effective against localized but not metastatic tumors. Single (FIG. 16A) or contralateral (FIG. 16B) B16/F10 tumors were established in syngeneic mice. Tumors on the left flank were then treated with either control virus (vGFP) or vPD1. Tumors on the right flank in contralateral model were left untreated. Responsiveness of individual tumors and overall survival were then monitored.

FIGS. 17A-17D—MYXV expressing both soluble PD1 and IL12 is highly effective against metastatic disease. (FIG. 17A) Genomic structure of viruses expressing both soluble PD1 and proinflammatory cytokines. Contralateral LLC tumors were established in syngeneic mice. Tumors on the left flank were then treated as indicated and tumors on the right flank were left untreated. (FIG. 17B) Responsiveness of individual tumors and (FIG. 17C) overall survival were then monitored. (FIG. 17D) Picture of mouse bearing bulky, contralateral LLC tumors treated as above.

FIGS. 18A-18C—vPD1/IL12 is effective against metastatic lung cancer. (FIG. 18A) Contralateral LLC tumors were established in syngeneic mice. Tumors on the left flank were then treated as indicated and tumors on the right flank were left untreated. (FIG. 18B) Responsiveness of individual tumors and (FIG. 18C) overall survival were then monitored.

FIGS. 19A-19C—vPD1/IL12 is effective against metastatic melanoma. (FIG. 19A) Contralateral B16/F10 tumors were established in syngeneic mice. Tumors on the left flank were then treated as indicated and tumors on the right flank were left untreated. (FIG. 19B) Responsiveness of individual tumors and (FIG. 19C) overall survival were then monitored.

FIGS. 20A-20C—vPD1/IL12 is effective against spontaneously metastatic breast cancer. (FIG. 20A) Single 4T1 tumors were established in syngeneic mice and allowed to establish and metastasize. Primary tumors were then treated as indicated. (FIG. 20B) Responsiveness of individual tumors and (FIG. 20C) overall survival were then monitored.

FIG. 21—Elements for use in multi-cistronic expression constructs.

FIG. 22—Proposed Bicistronic vPD1/IL12 Constructs (6 potential Constructs).

FIG. 23—Proposed Triply Recombinant vPD1/IL12/GeneX Constructs based on vPD1-bicistronic-IL12+Gene X Method (90 potential constructs for x=15).

FIG. 24—Proposed Triply Recombinant vPD1/GeneX/IL12 Constructs based on vPD1-bicistronic-GeneX+IL12 Method (90 potential constructs for x=15).

FIG. 25—Analysis of selected bicistronic elements. Plasmids encoding five previously proposed biscistronic constructs (vPBS-135-GFP-PD1-E2A-IL12-136, vPBS-135-GFP-PD1-F2A-IL12-136, vPBS-135-GFP-PD1-P2A-IL12-136, vPBS-135-GFP-PD1-HCV^{IRES}-IL12-136, vPBS-135-GFP-PD1-pcDNA3^{IRES}-IL12-136; see FIG. 22) were transfected into BSC40 cells. 24 hours after transfection, cells were infected with myxoma virus (Strain Lausanne). 48 hours after infection, both the cells and supernatant were harvested. Samples were subsequently analyzed for expression of both the PD1 and IL12 transgenes via western blot.

### DESCRIPTION OF ILLUSTRATIVE EMBODIMENTS

A major inhibitory pathway present in tumor microenvironments are the PD1-PDL1 checkpoint in which PDL1 expressed on tumor cells binds to PD1 on anti-tumor T cells resulting in T cell exhaustion. Current methods to overcome these pathways include systemic injection of antibodies which block the PD1-PDL1 interaction; however, these systemic treatments are costly, time consuming and associated with low response rates and noticeable toxicities.

Certain embodiments of the present disclosure provide compositions and methods for targeting the PD1-PDL1 pathway in cancer. In some aspects, a recombinant oncolytic myxoma virus is provided, which has been engineered to express the extracellular portion of the human PD1 protein along with IL-12. In certain aspects, the oncolytic myxoma virus is a replication competent virus such as myxoma virus. In particular, the extracellular region of PD1 and IL-12 can be encoded by one or more expression cassettes that is integrated into a region of the viral genome that is not necessary for replication. In the present studies, the oncolytic myxoma virus provided tumor inhibition that can significantly improve outcomes during oncolytic virotherapy. Additional variations where the oncolytic myxoma virus further contains an additional cytokine/chemokine other than IL-2 or a tumor antigen are also proposed.

Accordingly, further embodiments of the present disclosure provide methods of cancer treatment comprising administering the recombinant oncolytic myxoma virus expressing PD1, IL-12 and either a further chemokine/cytokine or a tumor antigen are also provided. Thus, the present aspects of the disclosure provide methods and compositions for a therapy targeting the PD1-PDL1 pathway, in combination with cytokine therapy, and optionally a tumor antigen vaccination with a low toxicity and high response rate.

### I. Definitions

The term “oncolytic virus,” as used herein, refers to a virus capable of selectively replicating in and slowing the growth or inducing the death of a cancerous or hyperproliferative cell, either in vitro or in vivo, while having no or minimal effect on normal cells. Exemplary oncolytic viruses include vesicular stomatitis virus (VSV), Newcastle disease virus (NDV), herpes simplex virus (HSV), reovirus, measles virus, retrovirus, influenza virus, Sinbis virus, vaccinia virus, and adenovirus.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The term “innate immunity” or “innate immune response” refers to the repertoire of host defenses, both immunological and nonimmunological, that exist prior to or independent of exposure to specific environmental antigens, such as a microorganism or macromolecule, etc. For example, the first host immune response to an antigen involves the innate immune system.

The term “immunogen” or “antigen,” as used herein, refers to an agent that is recognized by the immune system when introduced into a subject and is capable of eliciting an immune response. In certain embodiments, the immune response generated is an innate cellular immune response and the recombinant oncolytic viruses of the instant disclosure are capable of suppressing or reducing the innate cellular immune response.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences.

As used herein the term “multiplicity of infection” (MOI) means the number of infectious virus particles added per cell.

### II. Oncolytic Myxoma Viruses

Myxoma virus (MYVX) is a member of the Poxviridae family and prototype for the genus Leporipoxvirus. It is pathogenic only for European rabbits (Oryctolagus cuniculus), in which it causes a lethal disease called myxomatosis, and for two North American species, Sylvilagus audubonni and Sylvilagus nuttalli, in which it causes a less severe disease. Myxoma virus replicates exclusively in the cytoplasm of the host cell, and its genome encodes 171 open reading frames (Smallwood et al., 2010). A number of these genes encode proteins that can interfere with or modulate host defense mechanisms, and several show promise in a clinical setting.

Like other members of the poxvirus family, the myxoma virus genome consists of a single double stranded DNA (dsDNA), the central part of the which encodes approximately 100 essential genes that are conserved among the members of poxvirus genera. The rest of the genes, including two copies each of the 12 genes that map within the terminal inverted repeats, encode proteins that interfere with and modulate host defense mechanisms. A number of these proteins share a sequence similarity with host cellular genes, suggesting a coevolutionary path (Johnston and McFadden, 2003). Some, called viroceptors, are secreted and able to bind specific ligands such as TNF, for example. Others, known as virokines, are also secreted, and imitate host immune inhibitors, while viromitigators function as host range factors that inhibit apoptosis (Johnston and McFadden, 2003; Kerr and McFadden, 2002). These characteristics give myxoma virus possible utility in a number of therapeutic settings. One of the myxoma virus-encoded immunomodulatory proteins, Serp-1, is in clinical trials for acute unstable coronary syndromes (e.g., unstable angina and small heart attacks). The M-T7 protein of myxoma virus, a secreted glycoprotein that inhibits rabbit γ interferon, has also been shown to inhibit inflammatory responses in rabbit models of balloon angioplasty injury to arteries (Liu et al., 2000), and it is likely that a variety of other immunomodulatory proteins can be developed as anti-inflammatory or anti-immune therapeutics.

Myxoma virus has been shown to productively infect a variety of human cancer cell lines originated from a diverse group of tissues (Sypula et al., 2004), and therefore has the potential for development as an oncolytic virus useful in treatment against a variety of cancers. Wildtype myxoma virus can selectively infect and kill cells, including human cells, which have a deficient innate anti-viral response, for example, cells that are non-responsive to interferon, as described in the application PCT/CA2004/000341, which is herein fully incorporated by reference. Furthermore, myxoma virus is adept at evading and interfering with the host immune response and might serve as a source of immunomodulatory proteins that can be used as therapeutic agents in a variety of clinical settings (Lucas and McFadden, 2004). Additionally, although myxoma virus is not infectious in humans, it is able to productively infect a number of human cancer cell lines, but not normal human cells, and has also been shown to increase survival time in mouse models of human glioma. These characteristics suggest that myxoma virus could prove to be a viable therapeutic agent in a variety of clinical settings, including as an anti-inflammatory or anti-immune therapy, or as an oncolytic agent.

Myxoma virus has established oncolytic potential against a variety of malignancies including myeloma, melanoma, glioblastoma, pancreatic cancer, and others. The virus is thought to exhibit anti-tumor effects through two distinct mechanisms. First, the virus directly infects and kills tumor cells. Second, viral infection of tumor cells induces a secondary anti-tumor immune response. While the combination of these mechanisms is effective at debulking primary tumors, it often fails to produce long-term cures due to immune inhibition within the tumor microenvironment.

The myxoma virus of the present disclosure can be attenuated to enhance anti-tumor activity. For example, the myxoma virus can be genetically modified to inactivate one or more genes. In particular, myxoma virus that does not express functional M135R is useful for treatment of cells having a deficient innate anti-viral response, including for oncolytic studies, since this virus provides a safer alternative for oncolytic viral therapy as no unusual containment strategies should be needed for patients undergoing treatment (U.S. Patent Publication No. 20090035276, incorporated herein by reference). In certain aspects, the myxoma virus is an attenuated strain of myxoma virus such as the SG33 strain (U.S. Pat. No. 8,613,915, incorporated herein by reference). An attenuated myxoma virus which can be used in accordance with the disclosure may be obtained from a virulent wild-type myxoma virus, especially by deletion of one or more of the genes M151R, M152R, M153R, M154L, M156R, and M001R, and preferably by the additional deletion of one or more of the genes M008.1R, M008R, M007R, M006R, M005R, M004.1R, M004R, M003.2R, M003.1R, and M002R.

Myxoma virus can be propagated in a number of cell lines, including adherent cells and suspension cultures, and minimal purification is required. For example, myxoma virus can grow in several cell lines, including RK13 (rabbit kidney epithelial), BHK-21 (baby hamster kidney), BGMK (Buffalo green monkey kidney), Vero (African green monkey kidney epithelial), BSC-40 (African green monkey kidney), and CV-1 (African green monkey kidney fibroblast) cells. Minimal purification is needed to provide a stock that is appropriate for both in vitro and in vivo work. Protocols for propagating, purifying, and quantifying stocks of myxoma virus are known in the art (Smallwood et al., 2010, incorporated herein by reference).

B. Recombinant Oncolytic Viruses

The recombinant virus can be constructed by procedures known in the art to generate recombinant viruses. An expression cassette encoding PD1, such as mutant PD1, is inserted into the genome of an oncolytic virus at a region nonessential for viral replication. For example, the expression cassette can be integrated in myxoma virus at an intergenic region, such as between the M135 and M136 open reading frames. The recombinant virus can comprise an expression cassette comprising a nucleotide sequence which is at least about 50%, 55%, 60%, 65%, 70%, 75%, 80%, 85%, 90%, 91%, 92%, 93%, 94%, 95%, 96%, 97%, 98%, 99% or more identical to the nucleotide sequence (e.g., to the entire length of the nucleotide sequence) of the extracellular portion of human PD1, which is shown in SEQ ID NO: 3. The nucleotide sequence of SEQ ID NO: 3 can be optimized for expression in the recombinant virus, for example, through codon optimization. The expression cassette can encode for soluble TIM3 (SEQ ID NO: 11) or sequence with at least 50%, 55%, 60%, 65%, 70%, 75%, 80%, 85%, 90%, 91%, 92%, 93%, 94%, 95%, 96%, 97%, 98%, 99% or more identical to SEQ ID NO: 11).

Homologous recombination (HR), also known as general recombination, is a type of genetic recombination used in all forms of life in which nucleotide sequences are exchanged between two similar or identical strands of DNA. The technique has been the standard method for genome engineering in mammalian cells since the mid-1980s. The process involves several steps of physical breaking and the eventual rejoining of DNA. This process is most widely used to repair potentially lethal double-strand breaks in DNA. In addition, homologous recombination produces new combinations of DNA sequences during meiosis, the process by which eukaryotes make germ cells like sperm and ova. These new combinations of DNA represent genetic variation in offspring which allow populations to evolutionarily adapt to changing environmental conditions over time. Homologous recombination is also used in horizontal gene transfer to exchange genetic material between different strains and species of bacteria and viruses. Homologous recombination is also used as a technique in molecular biology for introducing genetic changes into target organisms.

Expression cassettes included in vectors useful in the disclosure preferably contain (in a 5′-to-3′ direction) a eukaryotic transcriptional promoter operably linked to a protein-coding sequence. Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., β actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a synthetic early/late (sE/L) poxvirus promoter (see, e.g., the promoter of the construct to SEQ ID NO: 10).

The expression cassette is introduced to cells which are then infected with the unmodified oncolytic virus to produce the recombinant virus. Introduction of the expression cassette into cells may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

The recombinant virus is then purified from the cells such as by a selectable marker. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker. Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. The marker used is not believed to be important, so long as it is capable of being expressed simultaneously with the nucleic acid encoding a gene product. For example, the recombinant oncolytic virus can be untagged or express fluorescent proteins such as green fluorescent protein (GFP), red fluorescent protein (RFP), tomato Red (tdRed), or other fluorescent proteins. Further examples of selection and screenable markers are well known to one of skill in the art.

The transgene expressing tomato red fluorescent (tdTr), which serves as a fluorescent marker for myxoma replication in vitro and in vivo, has been described in Liu et al. (2009) J. Virology 83:5933-5938. Liu observed that a myxovirus expressing IL-15 fused to tdTr (vMyx-IL-15-tdTr) was significantly attenuated and failed to induce lethal myxomatosis in rabbits. The construct secreted IL-15 and supported normal virus replication. Thus, Liu concluded that vMyx-IL-15-tdTr was a safe candidate for in vivo animal studies of oncolytic virotherapy, and tdTr is a suitable marker for use in recombinant myxovirus.

If desired, one or more genetic elements, such as transgenes expressing fluorescent markers, can be excised from a viral transposon, using methods known in the art, such as Flp recombinase or Cre-lox recombination-based systems.

C. Polycystonic Expression Cassettes

The present application contemplates the insertion of multiple heterologous sequences into the myxoma virus vectors. This may involve inserting different sequences at different myxoma genomic locations, inserting different sequences at the same myxoma genomic location, or both. When sequences are inserted at the same genomic location, they may be constructed such that a polycistronic message is generated, i.e., a single mRNA transcript encoding separate coding regions.

In certain embodiments, the use of internal ribosome entry sites (IRES) elements are used to create polycistronic, messages. IRES elements are able to bypass the ribosome scanning model of 5′-methylated Cap-dependent translation and begin translation at internal sites. Examples of IRES elements include both mammalian and viral IRESs. For example, members of the picornavirus family (poliovirus, rhinovirus and encephalomyocarditis) as well as a large number of other viruses, such as hepatitis C virus (HCV) encode IRES elements. Example IRESs for use according to the embodiments include, but are not limited to the IRES from eIF4G, BCL2, BiP, c-IAP1, poliovirus (PV), encephalomyelocarditis virus (EMCV), classical swine-fever virus (CSFV), foot-and-mouth disease virus (FMDV), human immunodeficiency virus (HIV), bovine viral diarrhoea virus (BVDV), HCV and cricket paralysis virus (CrPV). A comprehensive list of IRES elements can be found on the world wide web at iresite.org, incorporated herein by reference.

IRES elements can be linked to heterologous open reading frames. Multiple open reading frames can be transcribed together, each separated by an IRES, thereby creating polycistronic messages. By virtue of the IRES element, each open reading frame is accessible to ribosomes for efficient translation. Multiple genes can be efficiently expressed using a single promoter/enhancer to transcribe a single message.

Another approach to employing dicistronic or polycistronic messages involves expressing as a polyprotein. In some aspects, the proteins of the polyprotein are separated by protease cleavage sites. In certain aspects, the protease cleavage sites are cleaved by a protease that is active in target cell of the oncolytic virus. In certain aspects, the protease cleavage site can be a self-cleaving peptide, such as the 2A self-cleaving peptides, or 2A peptides. These peptides are members of a class of 18-22 aa-long peptides that can induce the cleaving of the recombinant protein in cell. 2A peptides are derived from the 2A region in the genome of certain viruses. The members of 2A peptides are named after the virus in which they have been first described. For example, F2A, the first described 2A peptide, is derived from foot-and-mouth disease virus. Four members of 2A peptides family are frequently used. They are P2A, E2A, F2A and T2A. F2A is derived from foot-and-mouth disease virus 18; E2A is derived from equine rhinitis A virus; P2A is derived from porcine teschovirus-1 2A; T2A is derived from Thoseaasigna virus 2A. Example sequences are shown below, with the GSG being an optional sequence that can improve efficiency.

T2A:
(SEQ ID NO: 15)
(GSG)EGRGSLLTCGDVEENPGP
P2A:
(SEQ ID NO: 16)
(GSG)ATNFSLLKQAGDVEENPGP
E2A:
(SEQ ID NO: 17)
(GSG)QCTNYALLKLAGDVESNPGP
F2A:
(SEQ ID NO: 18)
(GSG)VKQTLNFDLLKLAGDVESNPGP

The 2A-peptide-mediated cleavage commences after the translation. The cleavage is trigged by breaking of peptide bond between the Proline (P) and Glycine (G) in C-terminal of 2A peptide. The exact molecular mechanism of 2A-peptide-mediated cleavage is still unknown; however, it is believed to involve ribosomal “skipping” of glycyl-prolyl peptide bond formation rather than true proteolytic cleavage. Adding the sequence “GSG” (Gly-Ser-Gly) to the N-terminus of a 2A is a common modification.

D. PD-1

Programmed cell death protein 1, also known as PD-1 and CD279 (cluster of differentiation 279), is a protein found on the surface of cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells.

The amino acid sequence of the extracellular domain of human PD-1 is found at Uniprot Accession Number Q15116, SEQ ID NO: 4, and is 168 amino acids in length, which includes a 20 amino acid signal sequence which may be replaced by a different signal sequence, or omitted from the PD-1 sequences of the present disclosure, when not needed in order to direct secretion.

PD-1 is an immune checkpoint and guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells). PD-1 inhibitors, a new class of drugs that block PD-1, activate the immune system to attack tumors and are used to treat certain types of cancer.

The PD-1 protein in humans is encoded by the PDCD1 gene. PD-1 is a cell surface receptor that belongs to the immunoglobulin superfamily and is expressed on T cells and pro-B cells. PD-1 binds two ligands, PD-L1 and PD-L2. PD-1 is a type I membrane protein of 268 amino acids. PD-1 is a member of the extended CD28/CTLA-4 family of T cell regulators. The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates T-cell receptor TCR signals. This is consistent with binding of SHP-1 and SHP-2 phosphatases to the cytoplasmic tail of PD-1 upon ligand binding. In addition, PD-1 ligation up-regulates E3-ubiquitin ligases CBL-b and c-CBL that trigger T cell receptor down-modulation. PD-1 is expressed on the surface of activated T cells, B cells, and macrophages, suggesting that compared to CTLA-4, PD-1 more broadly negatively regulates immune responses.

PD-1 has two ligands, PD-L1 and PD-L2, which are members of the B7 family PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

Several lines of evidence suggest that PD-1 and its ligands negatively regulate immune responses. PD-1 knockout mice have been shown to develop lupus-like glomerulonephritis and dilated cardiomyopathy on the C57BL/6 and BALB/c backgrounds, respectively. In vitro, treatment of anti-CD3 stimulated T cells with PD-L1-Ig results in reduced T cell proliferation and IFN-γ secretion. IFN-γ is a key pro-inflammatory cytokine that promotes T cell inflammatory activity. Reduced T cell proliferation was also correlated with attenuated IL-2 secretion and together, these data suggest that PD-1 negatively regulates T cell responses.

Experiments using PD-L1 transfected DCs and PD-1 expressing transgenic (Tg) CD4⁺ and CD8⁺ T cells suggest that CD8⁺ T cells are more susceptible to inhibition by PD-L1, although this could be dependent on the strength of TCR signaling. Consistent with a role in negatively regulating CD8⁺ T cell responses, it has been shown the PD-1-PD-L1 interaction inhibits activation, expansion and acquisition of effector functions of virus specific CD8⁺ T cells, which can be reversed by blocking the PD-1-PD-L1 interaction.

Expression of PD-L1 on tumor cells inhibits anti-tumor activity through engagement of PD-1 on effector T cells. Expression of PD-L1 on tumors is correlated with reduced survival in esophageal, pancreatic and other types of cancers, highlighting this pathway as a target for immunotherapy. Triggering PD-1, expressed on monocytes and upregulated upon monocytes activation, by its ligand PD-L1 induces IL-10 production which inhibits CD4 T-cell function.

In mice, expression of this gene is induced in the thymus when anti-CD3 antibodies are injected and large numbers of thymocytes undergo apoptosis. Mice deficient for this gene bred on a BALB/c background developed dilated cardiomyopathy and died from congestive heart failure. These studies suggest that this gene product may also be important in T cell function and contribute to the prevention of autoimmune diseases. Overexpression of PD1 on CD8+ T cells is one of the indicators of T-cell exhaustion (e.g., in chronic infection or cancer).

PD-L1, the primary ligand for PD1, is highly expressed in several cancers and hence the role of PD1 in cancer immune evasion is well established. Monoclonal antibodies targeting PD-1 that boost the immune system are being developed for the treatment of cancer. Many tumor cells express PD-L1, an immunosuppressive PD-1 ligand; inhibition of the interaction between PD-1 and PD-L1 can enhance T-cell responses in vitro and mediate preclinical antitumor activity. This is known as immune checkpoint blockade.

Combination therapy using both anti-PD1 along with anti-CTLA4 therapeutics have emerged as important tumor treatments within the field of checkpoint inhibition. A combination of PD1 and CTLA4 antibodies has been shown to be more effective than either antibody alone in the treatment of a variety of cancers. The effects of the two antibodies do not appear to be redundant. Anti-CTLA4 treatment leads to an enhanced antigen specific T cell dependent immune reaction while anti-PD-1 appears to reactivate CD8+ T cells ability to lyse cancer cells.

In clinical trials, combination therapy has been shown to be effective in reducing tumor size in patients that are unresponsive to single co-inhibitory blockade, despite increasing levels of toxicity due to anti-CTLA4 treatment. A combination of PD1 and CTLA4 induced up to a ten-fold higher number of CD8+ T cells that are actively infiltrating the tumor tissue. The authors hypothesized that the higher levels of CD8+ T cell infiltration was due to anti-CTLA-4 inhibited the conversion of CD4 T cells to T regulator cells and further reduced T regulatory suppression with anti-PD-1. This combination promoted a more robust inflammatory response to the tumor that reduced the size of the cancer. Most recently, the FDA has approved a combination therapy with both anti-CTLA4 (ipilimumab) and anti-PD1 (nivolumab) in October 2015.

The molecular factors and receptors necessary making a tumor receptive to anti-PD1 treatment remains unknown. PD-L1 expression on the surface on cancer cells plays a significant role. PD-L1 positive tumors were twice as likely to respond to combination treatment. However patients with PD-L1 negative tumors also have limited response to anti-PD1, demonstrating that PD-L1 expression is not an absolute determinant of the effectiveness of therapy.

Higher mutational burden in the tumor is correlated with a greater effect of the anti-PD1 treatment. In clinical trials, patients who benefited from anti-PD1 treatment had cancers, such as melanoma, bladder cancer, and gastric cancer, that had a median higher average number of mutations than the patients who do did not respond to the therapy. However, the correlation between higher tumor burden and the clinical effectiveness of PD-1 immune blockade is still uncertain.

E. IL-12

Interleukin 12 (IL-12) is an interleukin that is naturally produced by dendritic cells, macrophages, neutrophils, and human B-lymphoblastoid cells (NC-37) in response to antigenic stimulation. IL-12 is composed of a bundle of four α helices. It is a heterodimeric cytokine encoded by two separate genes, IL-12A (p35) and IL-12B (p40). The active heterodimer (referred to as ‘p70’), and a homodimer of p40 are formed following protein synthesis. The amino acid sequence of human IL-12 α subunit is found at Uniprot Accession Number P29459, SEQ ID NO: 7, and is 219 amino acids in length, which includes a 22 amino acid signal sequence which may be replaced by a different signal sequence, or omitted from the IL-12 α subunit sequences of the present invention, when not needed in order to direct secretion. The amino acid sequence of human IL-12 β subunit is found at Uniprot Accession Number P29460, SEQ ID NO: 8, and is 328 amino acids in length, which includes a 22 amino acid signal sequence, which may be replaced by a different signal sequence, or omitted from the IL-12 β subunit sequences of the present invention, when not needed in order to direct secretion. The nucleotide sequences encoding IL-12 α and β subunits can be optimized for expression in the recombinant virus, for example, through codon optimization.

In certain embodiments, the IL-12 α subunit and IL-12 β subunit may be expressed as a fusion protein from a single DNA construct. In such cases, only a single signal peptide is required, preferably at the N-terminal end of the expressed fusion protein. In such cases, a flexible linker peptide may be used to join the IL-12 α subunit and IL-12 β subunits. Suitable linker peptide sequences are known in the art, and include, for example (GGGS)ₙ, where n=1 to 4.

IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ. T cells that produce IL-12 have a coreceptor, CD30, which is associated with IL-12 activity.

IL-12 plays an important role in the activities of natural killer cells and T lymphocytes. IL-12 mediates enhancement of the cytotoxic activity of NK cells and CD8+ cytotoxic T lymphocytes. There also seems to be a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signaling in NK cells Enhanced functional response is demonstrated by IFN-γ production and killing of target cells.

IL-12 also has anti-angiogenic activity, which means it can block the formation of new blood vessels. It does this by increasing production of interferon γ, which in turn increases the production of a chemokine called inducible protein-10 (IP-10 or CXCL10). IP-10 then mediates this anti-angiogenic effect. Because of its ability to induce immune responses and its anti-angiogenic activity, there has been an interest in testing IL-12 as a possible anti-cancer drug. However, it has not been shown to have substantial activity in the tumors tested to this date. There is a link that may be useful in treatment between IL-12 and the diseases psoriasis and inflammatory bowel disease.

IL-12 binds to the IL-12 receptor, which is a heterodimeric receptor formed by IL-12Rβ1 and IL-12Rβ2. IL-12Rβ2 is considered to play a key role in IL-12 function, since it is found on activated T cells and is stimulated by cytokines that promote Th1 cells development and inhibited by those that promote Th2 cells development. Upon binding, IL-12R-β2 becomes tyrosine phosphorylated and provides binding sites for kinases, Tyk2 and Jak2. These kinases are important in activating critical transcription factor proteins such as STAT4 that are implicated in IL-12 signaling in T cells and NK cells. This pathway is known as the JAK-STAT pathway.

IL-12 is linked with autoimmunity. Administration of IL-12 to people suffering from autoimmune diseases was shown to worsen the autoimmune phenomena. This is believed to be due to its key role in induction of Th1 immune responses. In contrast, IL-12 gene knock-out in mice or a treatment of mice with IL-12 specific antibodies ameliorated the disease.

Interleukin 12 (IL-12) is produced by activated antigen-presenting cells (dendritic cells, macrophages). It promotes the development of Th1 responses and is a powerful inducer of IFNγ production by T and NK cells.

A child with Bacillus Calmette-Guérin and Salmonella enteritidis infection was found to have a large homozygous deletion within the IL-12 p40 subunit gene, precluding expression of functional IL-12 p70 cytokine by activated dendritic cells and phagocytes. As a result, IFNγ production by the child's lymphocytes was markedly impaired. This suggested that IL-12 is essential for protective immunity to intracellular bacteria such as mycobacteria and Salmonella.

Support is lent to this idea by the observation that a receptor for IL-12 is important for IFNγ production by lymphocytes. T and NK cells from seven unrelated patients who had severe idiopathic mycobacterial and Salmonella infections failed to produce IFNγ when stimulated with IL-12. The patients were otherwise healthy. They were found to have mutations in the IL-12 receptor β1 chain, resulting in premature stop codons in the extracellular domain, resulting in unresponsiveness to this cytokine, again demonstrating IL-12's crucial role in host defense.

Defective Th1 and Th17 immune responses leading to chronic mucocutaneous candidiasis result from a mutation further downstream in the IL-12 signaling pathway. The trait was mapped to mutations in the STAT1 gene, which were associated with lower production of interferon-γ, IL-17, and IL-22 in response to IL-12 or IL-23 receptor associated Jak2 and Tyk2 activity.

F. Other Chemokines/Cytokines

IL-2. Interleukin-2 (IL-2) is an interleukin, a type of cytokine signaling molecule in the immune system. It is a protein that regulates the activities of white blood cells (leukocytes, often lymphocytes) that are responsible for immunity. IL-2 is part of the body's natural response to microbial infection, and in discriminating between foreign (“non-self”) and “self”. IL-2 mediates its effects by binding to IL-2 receptors, which are expressed by lymphocytes. The amino acid sequence of human IL-2 is found at Uniprot Accession Number P60568, SEQ ID NO: 6, and is 153 amino acids in length, which includes a 20 amino acid signal sequence, which may be replaced by a different signal sequence, or omitted from the IL-2 sequences of the present invention, when not needed in order to direct secretion. The nucleotide sequence encoding IL-2 can be optimized for expression in the recombinant virus, for example, through codon optimization.

In a preferred embodiment, the IL-2 useful in the present invention is the high affinity variant IL-2 amino acid sequence of SEQ ID NO: 9, which includes a 20 amino acid signal sequence, which may be replaced by a different signal sequence, and which also contains C-terminal His tag. Levin et al. (2012) Nature 484:529-533. Either or both of the signal sequence and His tag may be omitted, if not required for function of the IL-2.

IL-2 is a member of a cytokine family, each member of which has a four α helix bundle; the family also includes IL-4, IL-7, IL-9, IL-15 and IL-21. IL-2 signals through the IL-2 receptor, a complex consisting of three chains, termed α, β and γ. They chain is shared by all family members.

The IL-2 Receptor (IL-2R) a subunit has low affinity for its ligand but has the ability (when bound to the β and γ subunit) to increase the IL-2R affinity 100-fold. Heterodimerization of the β and γ subunits of IL-2R is essential for signaling in T cells.

Gene expression regulation for IL-2 can be on multiple levels or by different ways. One of the checkpoints is signaling through TCR receptor, antigen receptor of T-lymphocytes after recognizing MHC-peptide complex. Signaling pathway from TCR then goes through phospholipase-C (PLC) dependent pathway. PLC activates 3 major transcription factors and their pathways: NFAT, NFkB and AP-1. After costimulation from CD28 the optimal activation of expression of IL-2 and these pathways is induced.

At the same time Oct-1 is expressed. It helps the activation. Oct1 is expressed in T-lymphocytes and Oct2 is induced after cell activation. NFAT has multiple family members, all of them are located in cytoplasm and signaling goes through calcineurin, NFAT is dephosphorylated and therefore translocated to the nucleus. AP-1 is a dimer and is composed of c-Jun and c-Fos proteins. It cooperates with other transcription factors including NFkB and Oct. NFkB is translocated to the nucleus after costimulation through CD28. NFkB is a heterodimer and there are two binding sites on the IL-2 promoter.

IL-2 has essential roles in key functions of the immune system, tolerance and immunity, primarily via its direct effects on T cells. In the thymus, where T cells mature, it prevents autoimmune diseases by promoting the differentiation of certain immature T cells into regulatory T cells, which suppress other T cells that are otherwise primed to attack normal healthy cells in the body. IL-2 also promotes the differentiation of T cells into effector T cells and into memory T cells when the initial T cell is also stimulated by an antigen, thus helping the body fight off infections. Its expression and secretion is tightly regulated and functions as part of both transient positive and negative feedback loops in mounting and dampening immune responses. Through its role in the development of T cell immunologic memory, which depends upon the expansion of the number and function of antigen-selected T cell clones, it plays a key role in enduring cell-mediated immunity.

Aldesleukin is a form of recombinant interleukin-2. It is manufactured using recombinant DNA technology and is marketed as a protein therapeutic and branded as Proleukin. It has been approved by the Food and Drug Administration (FDA) and in several European countries for the treatment of cancers (malignant melanoma, renal cell cancer) in large intermittent doses and has been extensively used in continuous doses. Interking is a recombinant IL-2 with a serine at residue 125, sold by Shenzhen Neptunus.

Various dosages of IL-2 across the United States and across the world are used. The efficiency and side effects of different dosages is often a point of disagreement. Usually, in the U.S., the higher dosage option is used, affected by cancer type, response to treatment and general patient health. Patients are typically treated for five consecutive days, three times a day, for fifteen minutes. The following approximately 10 days help the patient to recover between treatments. IL-2 is delivered intravenously on an inpatient basis to enable proper monitoring of side effects.

A lower dose regimen involves injection of IL-2 under the skin typically on an outpatient basis. It may alternatively be given on an inpatient basis over 1-3 days, similar to and often including the delivery of chemotherapy. Intralesional IL-2 is commonly used to treat in-transit melanoma metastases and has a high complete response rate and is generally well-tolerated.

IL-2 has a narrow therapeutic window, and the level of dosing usually determines the severity of the side effects. Some common side effects include flu-like symptoms (fever, headache, muscle and joint pain, fatigue), nausea/vomiting, dry, itchy skin or rash, weakness or shortness of breath, diarrhea, low blood pressure, drowsiness or confusion, and loss of appetite. More serious and dangerous side effects sometimes are seen, such as capillary leak syndrome, breathing problems, serious infections, seizures, allergic reactions, heart problems or a variety of other possible complications.

Other Interleukins. IL-4, IL-15, IL-17, IL-18 (mutated form), IL-23, IL-35, and IL-36 are also contemplated for inclusion in the oncolytic myxoma virus vectors described herein.

Interleukin 4 (IL-4) is a cytokine that induces differentiation of naive helper T cells (Th0 cells) to Th2 cells. Upon activation by IL-4, Th2 cells subsequently produce additional IL-4 in a positive feedback loop. The cell that initially produces IL-4, thus inducing Th2 differentiation, has not been identified, but recent studies suggest that basophils may be the effector cell. It is closely related and has functions similar to interleukin 13. Interleukin 4 has many biological roles, including the stimulation of activated B-cell and T-cell proliferation, and the differentiation of B cells into plasma cells. It is a key regulator in humoral and adaptive immunity. IL-4 induces B-cell class switching to IgE, and up-regulates MHC class II production. IL-4 decreases the production of Th1 cells, macrophages, IFN-γ, and dendritic cell IL-12. Overproduction of IL-4 is associated with allergies.

The receptor for interleukin-4 is known as the IL-4Rα. This receptor exists in 3 different complexes throughout the body. Type 1 receptors are composed of the IL-4Rα subunit with a common γ chain and specifically bind IL-4. Type 2 receptors consist of an IL-4Rα subunit bound to a different subunit known as IL-13Rα1. These type 2 receptors have the ability to bind both IL-4 and IL-13, two cytokines with closely related biological functions.

IL-4 has a significant effect on tumor progression. Increased IL-4 production was found in breast, prostate, lung, renal cells and other types of cancer. Many overexpression of IL-4R has been found in many types of cancer. Renal cells and glioblastoma modify 10,000-13,000 receptors per cell depending on tumor type. IL-4 can primitively motivate tumor cells and increase their apoptosis resistance by increasing tumor growth.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (γ-C, CD132). IL-15 is secreted by mononuclear phagocytes (and some other cells) following infection by virus(es). This cytokine induces cell proliferation of natural killer cells; cells of the innate immune system whose principal role is to kill virally infected cells.

IL-15 was discovered in 1994 and characterized as T cell growth factor. Together with Interleukin-2 (IL-2), Interleukin-4 (IL-4), Interleukin-7 (IL-7), Interleukin-9 (IL-9), granulocyte colony-stimulating factor (G-CSF), and granulocyte-macrophage colony-stimulating factor (GM-CSF), IL-15 belongs to the four α-helix bundle family of cytokine. IL-15 is constitutively expressed by a large number of cell types and tissues, including monocytes, macrophages, dendritic cells (DC), keratinocytes, fibroblasts, myocyte and nerve cells. As a pleiotropic cytokine, it plays an important role in innate and adaptive immunity.

IL-15 regulates the activation and proliferation of T and natural killer (NK) cells. Survival signals that maintain memory T cells in the absence of antigen are provided by IL-15. This cytokine is also implicated in NK cell development. In rodent lymphocytes, IL-15 prevents apoptosis by inducing BCL2L1/BCL-x(L), an inhibitor of the apoptosis pathway.^{[10]} In humans with celiac disease IL-15 similarly suppresses apoptosis in T-lymphocytes by inducing Bcl-2 and/or Bcl-xL.

A hematopoietin receptor, the IL-15 receptor, that binds IL-15 propagates its function. Some subunits of the IL-15 receptor are shared in common with the receptor for a structurally related cytokine called Interleukin 2 (IL-2) allowing both cytokines to compete for and negatively regulate each other's activity. CD8+ memory T cell number is controlled by a balance between IL-15 and IL-2. When IL-15 binds its receptor, JAK kinase, STAT3, STAT5, and STAT6 transcription factors are activated to elicit downstream signaling events.

IL-15 and its receptor subunit alpha (IL-15Rα) are also produced by skeletal muscle in response to different exercise doses (myokine), playing significant roles in visceral (intra-abdominal or interstitial) fat reduction and myofibrillar protein synthesis (hypertrophy).

Interleukin 17A (IL-17 or IL-17A) is a pro-inflammatory cytokine. This cytokine is produced by a group of T helper cell known as T helper 17 cell in response to their stimulation with IL-23. The protein encoded by IL17A is a founding member of IL-17 family (see below). IL17 protein exhibits a high homology with a viral IL-17-like protein encoded in the genome of T-lymphotropic rhadinovirus Herpesvirus saimiri. In rodents, IL-17 is often referred to as CTLA8.

The biologically active IL-17 interacts with type I cell surface receptor IL-17R. In turn, there are at least three variants of IL-17R referred to as IL17RA, IL17RB, and IL17RC. After binding to the receptor, IL-17 activates several signalling cascades that, in turn, lead to the induction of chemokines. Acting as chemoattractants, these chemokines recruit the immune cells, such as monocytes and neutrophils to the site of inflammation. Typically, the signaling events mentioned above follow an invasion of the body by pathogens. Promoting the inflammation, IL-17 acts in concert with tumor necrosis factor and interleukin-1. Moreover, an activation of IL-17 signaling is often observed in the pathogenesis of various autoimmune disorders, such as psoriasis.

The IL-17 family comprises IL17A, IL-17B, IL-17C, IL-17D, IL-17E and IL-17F. IL-17E is also known as IL-25. All members of the IL-17 family have a similar protein structure. Their protein sequences contain four highly conserved cysteine residues. These conserved cysteine residues are critical to the right 3-dimensional shape of the entire protein molecule. To the reference, the members of IL-17 family do not exhibit a significant sequence homology with other cytokines. Among IL-17 family members, the IL-17F isoforms 1 and 2 (ML-1) have the highest sequence homology with IL-17A (55 and 40%, respectively). They follow by IL-17B, which has 29% similarity to IL-17A, IL-17D (25%), IL-17C (23%), and IL-17E (17%). In mammals, the sequences of these cytokines are highly conserved. For instance, the sequence homology between the corresponding human and mouse proteins is usually between 62-88%.

Numerous immune regulatory functions have been reported for the IL-17 family of cytokines, presumably due to their induction of many immune signaling molecules. The most notable role of IL-17 is its involvement in inducing and mediating proinflammatory responses. IL-17 is commonly associated with allergic responses. IL-17 induces the production of many other cytokines (such as IL-6, G-CSF, GM-CSF, IL-1β, TGF-β, TNF-α), chemokines (including IL-8, GRO-α, and MCP-1), and prostaglandins (e.g., PGE₂) from many cell types (fibroblasts, endothelial cells, epithelial cells, keratinocytes, and macrophages). IL-17 acts with IL-22 (produced mainly by T helper 22 cells in humans, but by T-helper 17 in mice) to induce expression of antimicrobial peptide by keratinocytes.

The release of cytokines causes many functions, such as airway remodeling, a characteristic of IL-17 responses. The increased expression of chemokines attracts other cells including neutrophils but not eosinophils. IL-17 function is also essential to a subset of CD4+ T-Cells called T helper 17 (Th17) cells. As a result of these roles, the IL-17 family has been linked to many immune/autoimmune related diseases including rheumatoid arthritis, asthma, lupus, allograft rejection, anti-tumor immunity and recently psoriasis and multiple sclerosis.

The IL-17 receptor family consists of five, broadly distributed receptors (IL-17RA, B, C, D and E) that present with individual ligand specificities. Within this family of receptors, IL-17RA is the best-described. IL-17RA binds both IL-17A and IL-17F and is expressed in multiple tissues: vascular endothelial cells, peripheral T cells, B cell lineages, fibroblast, lung, myelomonocytic cells, and marrow stromal cells. Signal transduction for both IL-17A and IL-17F requires the presence of a heterodimeric complex consisting of both IL-17RA and IL-17RC and the absence of either receptor results in ineffective signal transduction. This pattern is reciprocated for other members of the IL-17 family such as IL-17E, which requires an IL-17RA-IL-17RB complex (also known as IL-17Rh1, IL-17BR or IL-25R) for effective function.

Another member of this receptor family, IL-17RB, binds both IL-17B and IL-17E. Furthermore, it is expressed in the kidney, pancreas, liver, brain, and intestine. IL-17RC is expressed by the prostate, cartilage, kidney, liver, heart, and muscle, and its gene may undergo alternate splicing to produce a soluble receptor in addition to its cell membrane-bound form. In similar manner, the gene for IL-17RD may undergo alternative splicing to yield a soluble receptor. This feature may allow these receptors to inhibit the stimulatory effects of their yet-undefined ligands. The least-described of these receptors, IL-17RE, is known to be expressed in the pancreas, brain, and prostate.

Signal transduction by these receptors is as diverse as their distribution. These receptors do not exhibit a significant similarity in extracellular or intracellular amino acid sequence when compared to other cytokine receptors. Transcription factors such as TRAF6, JNK, Erk1/2, p38, AP-1 and NF-κB have been implicated in IL-17 mediated signaling in a stimulation-dependent, tissue-specific manner Other signaling mechanisms have also been proposed, but more work is needed to fully elucidate the true signaling pathways used by these diverse receptors.

Interleukin-18 (IL18, also known as interferon-gamma inducing factor) is a protein which in humans is encoded by the IL18 gene. The protein encoded by this gene is a proinflammatory cytokine. Many cell types, both hematopoietic cells and non-hematopoietic cells, have the potential to produce IL-18. It was first described in 1989 as a factor that induced interferon-γ (IFN-γ) production in mouse spleen cells. Originally, IL-18 production was recognized in Kupffer cells, liver-resident macrophages. However, IL-18 is constitutively expressed in non-hematopoietic cells, such as intestinal epithelial cells, keratinocytes, and endothelial cells. IL-18 can modulate both innate and adaptive immunity and its dysregulation can cause autoimmune or inflammatory diseases.

IL-18 receptor consists of the inducible component IL-18Rα, which binds the mature IL-18 with low affinity and the constitutively expressed co-receptor IL-18Rβ. IL-18 binds the ligand receptor IL-18Rα, inducing the recruitment of IL-18Rβ to form a high affinity complex, which signals through the toll/interleukin-1 receptor (TIR) domain. This signaling domain recruits MyD88 adaptor protein that activates proinflammatory programs and NF-κB pathway. The activity of IL-18 can be suppressed by extracellular interleukin 18 binding protein (IL-18BP) that binds soluble IL-18 with a higher affinity than IL-18Ra thus prevents IL-18 binding to IL-18 receptor. IL-37 is another endogenous factor that suppresses the action of IL-18. IL-37 has high homology with IL-18 and can bind to IL-18Rα, which then forms a complex with IL-18BP, thereby reduces the activity of IL-18. Moreover, IL-37 binds to single immunoglobulin IL-1 receptor related protein (SIGIRR), also known as IL-1R8 or TIRE, which forms a complex with IL-18Ra and induces an anti-inflammatory response. The IL-37/IL-18Ra/IL-1R8 complex activates the STAT3 signaling pathway, decreases NF-κB and AP-1 activation and reduces IFNγ production. Thus, IL-37 and IL-18 have opposing roles and IL-37 can modulate pro-inflammatory effects of IL-18.

IL-18 belongs to the IL-1 superfamily and is produced mainly by macrophages but also other cell types, stimulates various cell types and has pleiotropic functions. IL-18 is a proinflammatory cytokine that facilitates type 1 responses. Together with IL-12, it induces cell-mediated immunity following infection with microbial products like lipopolysaccharide (LPS). IL-18 in combination with IL12 acts on CD4, CD8 T cells and NK cells to induce IFNγ production, type II interferon that plays an important role in activating the macrophages or other cells. The combination of this IL-18 and IL-12 has been shown to inhibit IL-4 dependent IgE and IgG1 production and enhance IgG2a production in B cells. Importantly, without IL-12 or IL-15, IL-18 does not induce IFNγ production, but plays an important role in the differentiation of naive T cells into Th2 cells and stimulates mast cells and basophils to produce IL-4, IL-13, and chemical mediators such as histamine.

Interleukin-23 (IL-23) is a heterodimeric cytokine composed of an IL12B (IL-12p40) subunit (that is shared with IL12) and the IL23A (IL-23p19) subunit. A functional receptor for IL-23 (the IL-23 receptor) has been identified and is composed of IL-12R (31 and IL-23R.

IL-23 is a proinflammatory cytokine. IL-23 has been shown to be a key cytokine for Th17 maintenance and expansion. Th17 are polarised by IL-6 and TGF-β which activate Th17 transcription factor RORγt. IL-23 stabilises RORγt and thus enables Th17 to properly function and release their effector cytokines such as IL-17, IL-21, IL-22 and GM-CSF which mediate protection against extracellular parasites (fungi and bacteria) and participate in barrier immunity. Similar effects as IL-23 has on Th17 cells were described on type 3 innate lymphoid cells which actively secrete Th17 cytokines upon IL-23 stimulation. NK cells express IL-23 receptor too. They respond with increased IFN-γ secretion and enhanced antibody-dependent cellular cytotoxicity. IL-23 also induces proliferation of CD4 memory T cells (not naïve cells). Along with mentioned proinflammatory effects IL-23 promotes angiogenesis.

IL-23 is mainly secreted by activated dendritic cells, macrophages or monocytes. Secretion is stimulated by an antigen stimulus recognized by a pattern recognition receptor. IL-23 imbalance and increase is associated with autoimmune and cancerous diseases. It is thus a target for therapeutic research.

Prior to the discovery of IL-23, IL-12 had been proposed to represent a key mediator of inflammation in mouse models of inflammation. However, many studies aimed at assessing the role of IL-12 had blocked the activity of IL-12p40 and were therefore not as specific as thought. Studies which blocked the function of IL-12p35 did not produce the same results as those targeting IL-12p40 as would have been expected if both subunits formed part of IL-12 only.

The discovery of an additional potential binding partner for IL-12p40 led to a reassessment of this role for IL-12. Seminal studies in experimental autoimmune encephalomyelitis, a mouse model of multiple sclerosis, showed that IL-23 was responsible for the inflammation observed, not IL-12 as previously thought. Subsequently, IL-23 was shown to facilitate development of inflammation in numerous other models of immune pathology where IL-12 had previously been implicated including models of arthritis, intestinal inflammation, and psoriasis. Ustekinumab, a monoclonal antibody directed against this cytokine, is used clinically to treat certain autoimmune conditions.

IL-23 heterodimer binds the receptor complex—p19 subunit binds IL-23R while p40 subunit binds IL-12RB1 which leads to recruitment of Janus kinase 2 and Tyrosine kinase 2 kinases. Janus kinase 2 and Tyrosine kinase 2 transduce the signal and phosphorylate STAT3 and STAT4. STATs dimerise and activate transcription of target genes in nucleus. STAT3 is responsible for key Th17 development attributes like RORγt expression or transcription of Th17 cytokines.

Interleukin 35 (IL-35) is a recently discovered cytokine from the IL-12 family IL-35 is produced by wide range of regulatory lymphocytes and plays a role in immune suppression. IL-35 is a dimeric protein composed of IL-12α and IL-27β chains, which are encoded by two separate genes called IL12A and EBI3, respectively. IL-35 receptor consists of IL-12Rβ2 (part of the IL-12R) and gp130 (part of IL-27R) chains. Compared to these two related interleukins, IL-35 is also able to signal through only one of the aforementioned chains. This was proven in vivo when absence of either of the receptor chains did not influence effects of IL-35.

Secreted by regulatory T-cells (T_{regs}), regulatory B-cells (B_{regs}) or even CD8+ regulatory T cells, IL-35 suppresses inflammatory responses of immune cells. IL-35 is not constitutively expressed in tissues, but the gene encoding IL-35 is transcribed by vascular endothelial cells, smooth muscle cells and monocytes after activation with proinflammatory stimuli. IL-35 has selective activities on different T-cell subsets; it induces proliferation of T_{reg} cell populations but reduces activity of Tₕ17 cell populations.

Interleukin 36, or IL-36, is a group of cytokines in the IL-1 family with pro-inflammatory effects. The role of IL-36 in inflammatory diseases is under investigation. There are four members of the IL-36 family which bind to the IL-36 receptor (IL1RL2/IL-1Rrp2/IL-36 receptor dimer) with varying affinities. IL36A, IL36B, and IL36G are IL-36 receptor agonists. IL36RA is an IL-36 receptor antagonist, inhibiting IL-36R signaling. The agonists are known to activate NF-κB and mitogen-activated protein kinases to induce various proinflammatory mediators.^{[3]} Binding of the IL-36R agonists to IL-1Rp2 recruits IL-1RAcP, activating the signaling pathway. IL-36Ra binds to IL-36R, preventing the recruitment of IL-1RAcP.

It has been found to activate T cell proliferation and release of IL-2. Before the functions of the IL-36 cytokines were determined, they were named as derivatives of IL-1F. The genes encoding for the IL-36 cytokines are found on chromosome 2q13.

Due to their predominant expression in epithelial tissues, IL-36 cytokines are believed to play a significant role in the pathogenesis of skin diseases, especially that of psoriasis. IL-36 has also been linked to psoriatic arthritis, systemic lupus erythematosus, inflammatory bowel disease, ulcerative colitis, Crohn's disease, and Sjögren's syndrome.

IL-36 must be cleaved at the N-terminus to become active, but the enzyme responsible for this is not known. IL-36 is expressed by many cell types, most predominately keratinocytes, respiratory epithelium, various nervous tissue, and monocytes.

IFNγ. Interferon gamma (IFNγ) is a dimerized soluble cytokine that is the only member of the type II class of interferons. The existence of this interferon, which early in its history was known as immune interferon, was first described as a product of human leukocytes stimulated with phytohemagglutinin, and as a product of antigen-stimulated lymphocytes. It was also shown to be produced in human lymphocytes. or tuberculin-sensitized mouse peritoneal lymphocytes challenged with PPD; the resulting supernatants were shown to inhibit growth of vesicular stomatitis virus. Those reports also contained the basic observation underlying the now widely employed interferon gamma release assay used to test for tuberculosis. In humans, the IFNγ protein is encoded by the IFNG gene.

IFNγ, or type II interferon, is a cytokine that is critical for innate and adaptive immunity against viral, some bacterial and protozoal infections. IFNγ is an important activator of macrophages and inducer of Class II major histocompatibility complex (MHC) molecule expression. Aberrant IFNγ expression is associated with a number of autoinflammatory and autoimmune diseases. The importance of IFNγ in the immune system stems in part from its ability to inhibit viral replication directly, and most importantly from its immunostimulatory and immunomodulatory effects. IFNγ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops. IFNγ is also produced by non-cytotoxic innate lymphoid cells (ILC).¹

IFNγ is secreted by T helper cells (specifically, Tₕ1 cells), cytotoxic T cells (T_{c} cells), macrophages, mucosal epithelial cells and NK cells. IFNγ is the only Type II interferon and it is serologically distinct from Type I interferons; it is acid-labile, while the type I variants are acid-stable.

IFNγ has antiviral, immunoregulatory, and anti-tumor properties.^{[19]} It alters transcription in up to 30 genes producing a variety of physiological and cellular responses. Among the effects are that it promotes NK cell activity, increases antigen presentation and lysosome activity of macrophages, activates inducible nitric oxide synthase (iNOS), induces the production of IgG2a and IgG3 from activated plasma B cells, causes normal cells to increase expression of class I MHC molecules as well as class II MHC on antigen-presenting cells—to be specific, through induction of antigen processing genes, including subunits of the immunoproteasome (MECL1, LMP2, LMP7), as well as TAP and ERAAP in addition possibly to the direct upregulation of MHC heavy chains and B2-microglobulin itself and promotes adhesion and binding required for leukocyte migration.

IFNγ is not approved yet for the treatment in any cancer immunotherapy. However, improved survival was observed when Interferon gamma was administrated to patients with bladder carcinoma and melanoma cancers. The most promising result was achieved in patients with stage 2 and 3 of ovarian carcinoma. In addition, it has been reported that mammalian glycosylation of recombinant human IFNγ, expressed in HEK293, improves its therapeutic efficacy compared to the unglycosylated form that is expressed in E. coli.

IFNβ. Interferon beta-1a (also interferon beta 1-alpha) is a cytokine in the interferon family used to treat multiple sclerosis (MS). It is produced by mammalian cells, while interferon beta-1b is produced in modified E. coli. Some claims have been made that Interferons produce about an 18-38% reduction in the rate of MS relapses. IFNβ has not been shown to slow the advance of disability. Interferons are not a cure for MS (there is no known cure); the claim is that interferons may slow the progress of the disease if started early and continued for the duration of the disease.

IFNβ balances the expression of pro- and anti-inflammatory agents in the brain and reduces the number of inflammatory cells that cross the blood brain barrier. Overall, therapy with interferon beta leads to a reduction of neuron inflammation. Moreover, it is also thought to increase the production of nerve growth factor and consequently improve neuronal survival. In vitro, IFNβ reduces production of Th17 cells which are a subset of T lymphocytes believed to have a role in the pathophysiology of MS.

A variety of commercial IFNβ forms are sold. Avonex was approved in the U.S. in 1996, and in Europe in 1997, and is registered in more than 80 countries worldwide. It is the leading MS therapy in the US, with around 40% of the overall market, and in Europe, with around 30% of the overall market. Avonex is sold in three formulations, a lyophilized powder requiring reconstitution, a pre-mixed liquid syringe kit, and a pen; it is administered once per week via intramuscular injection.

Rebif is a disease-modifying drug (DMD) used to treat multiple sclerosis in cases of clinically isolated syndromes as well as relapsing forms of multiple sclerosis and is similar to the IFNβ protein produced by the human body. It is co-marketed by Merck Serono and Pfizer in the U.S. under an exception to the Orphan Drug Act. Rebif is administered via subcutaneous injection three times per week and can be stored at room temperature for up to 30 days.

CinnoVex is the trade name of recombinant interferon beta-la, which is manufactured as biosimilar/biogeneric in Iran. It is produced in a lyophilized form and sold with distilled water for injection. There are several clinical studies to prove the similarity of CinnoVex and Avonex. A more water-soluble variant is currently being investigated.

Plegridy is the trade name of a pegylated form of Interferon beta-la. Plegridy's advantage is it only needs injecting once every two weeks.

Closely related to interferon beta-la is interferon beta-1b, which is also indicated for MS, but is formulated with a different dose and administered with a different frequency. Each drug has a different safety/efficacy profile. Interferon beta-1b is marketed only by Bayer in the U.S. as Betaseron, and outside the U.S. as Betaferon.

CCL5/RANTES. Chemokine (C—C motif) ligand 5 (also CCL5) is a protein which in humans is encoded by the CCL5 gene. It is also known as RANTES (regulated on activation, normal T cell expressed and secreted). CCL5 is an 8 kDa protein classified as a chemotactic cytokine or chemokine. CCL5 is chemotactic for T cells, eosinophils, and basophils, and plays an active role in recruiting leukocytes into inflammatory sites. With the help of particular cytokines (i.e., IL-2 and IFN-γ) that are released by T cells, CCL5 also induces the proliferation and activation of certain natural-killer (NK) cells to form CHAK (CC-Chemokine-activated killer) cells. It is also an HIV-suppressive factor released from CD8+ T cells. This chemokine has been localized to chromosome 17 in humans.

RANTES was first identified in a search for genes expressed “late” (3-5 days) after T cell activation. It was subsequently determined to be a CC chemokine and expressed in more than 100 human diseases. RANTES expression is regulated in T lymphocytes by Kruppel like factor 13 (KLF13). RANTES, along with the related chemokines MIP-1alpha and MIP-1beta, has been identified as a natural HIV-suppressive factor secreted by activated CD8+ T cells and other immune cells. Recently, the RANTES protein has been engineered for in vivo production by Lactobacillus bacteria, and this solution is being developed into a possible HIV entry-inhibiting topical microbicide.

cGAS. Cyclic GMP-AMP synthase (cGAS, cGAMP synthase), belonging to the nucleotidyltransferase family, is a cytosolic DNA sensor that activates a type-I interferon response. It is part of the cGAS-STING DNA sensing pathway. It binds to microbial DNA as well as self-DNA that invades the cytoplasm and catalyzes cGAMP synthesis. cGAMP then functions as a second messenger that binds to and activates the endoplasmic reticulum protein STING to trigger type-I IFNs production. Mice lacking cGAS were more vulnerable to lethal infection by DNA viruses. In addition, cGAS has been shown to be an innate immune sensor of retroviruses including HIV.

Ebola GP (aa1-298). Ebola virus (EBOV), a member of the Filoviridae family, causes severe disease with high fatality in humans. The RNA genome of EBOV encodes seven viral structural proteins including nucleoprotein (NP), and virion protein (VP) 35, VP40, glycoprotein (GP), VP30, VP24, and RNA-dependent RNA polymerase (L). The coding region for EBOV GP also gives rise to non-structural soluble GP (sGP) and shed GP, which is generated from the mature trimeric surface GP via proteolytic cleavage of the transmembrane region. GP is the only viral protein exposed on the surface of mature viral particles and associated with induction of protective immune responses. EBOV GP has been shown to induce pro-inflammatory cykines and chemokines via TLR4-mediated signaling including TNF-α, IL-1β, IL-6, MIP-1α, MCP-1 as well as the anti-inflammatory IL-10 molecule.

G. Tumor Antigens

A tumor antigen is an antigenic substance produced in tumor cells, i.e., it triggers an immune response in the host. Tumor antigens are useful tumor markers in identifying tumor cells with diagnostic tests and are potential candidates for use in cancer therapy. Normal proteins in the body are not antigenic because of self-tolerance, a process in which self-reacting cytotoxic T lymphocytes (CTLs) and autoantibody-producing B lymphocytes are called “centrally” in primary lymphatic tissue (BM) and “peripherally” in secondary lymphatic tissue (mostly thymus for T-cells and spleen/lymph nodes for B cells). Thus, any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

Initially tumor antigens were broadly classified into two categories based on their pattern of expression: Tumor-Specific Antigens (TSA), which are present only on tumor cells and not on any other cell, and Tumor-Associated Antigens (TAA), which are present on some tumor cells and also some normal cells. This classification, however, is imperfect because many antigens thought to be tumor-specific turned out to be expressed on some normal cells as well. The modern classification of tumor antigens is based on their molecular structure and source. Accordingly, they can be classified as follows:

Products of Mutated Oncogenes and Tumor Suppressor Genes

Products of Other Mutated Genes
Overexpressed or Aberrantly Expressed Cellular Proteins
Tumor Antigens Produced by Oncogenic Viruses
Oncofetal Antigens
Altered Cell Surface Glycolipids and Glycoproteins
Cell Type-Specific Differentiation Antigens

p53. Tumor protein p53, also known as p53, cellular tumor antigen p53, phosphoprotein p53, tumor suppressor p53, antigen NY-CO-13, or transformation-related protein 53 (TRP53), is any isoform of a protein encoded by homologous genes in various organisms, such as TP53 (humans) and Trp53 (mice). This homolog (originally thought to be, and often spoken of as, a single protein) is crucial in multicellular organisms, where it prevents cancer formation, and thus functions as a tumor suppressor. As such, p53 has been described as “the guardian of the genome” because of its role in conserving stability by preventing genome mutation. Hence, TP53 is classified as a tumor suppressor gene.

The name p53 was given in 1979 describing the apparent molecular mass; SDS-PAGE analysis indicates that it is a 53-kilodalton (kDa) protein. However, the actual mass of the full-length p53 protein (p53a) based on the sum of masses of the amino acid residues is only 43.7 kDa. This difference is due to the high number of proline residues in the protein, which slow its migration on SDS-PAGE, thus making it appear heavier than it actually is. In addition to the full-length protein, the human TP53 gene encodes at least 15 protein isoforms, ranging in size from 3.5 to 43.7 kDa. All these p53 proteins are called the p53 isoforms. The TP53 gene is the most frequently mutated gene (>50%) in human cancer, indicating that the TP53 gene plays a crucial role in preventing cancer formation. TP53 gene encodes proteins that bind to DNA and regulate gene expression to prevent mutations of the genome.

In humans, the TP53 gene is located on the short arm of chromosome 17 (17p13.1). The gene spans 20 kb, with a non-coding exon 1 and a very long first intron of kb. The coding sequence contains five regions showing a high degree of conservation in vertebrates, predominantly in exons 2, 5, 6, 7 and 8, but the sequences found in invertebrates show only distant resemblance to mammalian TP53. TP53 orthologs have been identified in most mammals for which complete genome data are available.

In humans, a common polymorphism involves the substitution of an arginine for a proline at codon position 72. Many studies have investigated a genetic link between this variation and cancer susceptibility; however, the results have been controversial. For instance, a meta-analysis from 2009 failed to show a link for cervical cancer. A 2011 study found that the TP53 proline mutation did have a profound effect on pancreatic cancer risk among males. A study of Arab women found that proline homozygosity at TP53 codon 72 is associated with a decreased risk for breast cancer. One study suggested that TP53 codon 72 polymorphisms, MDM2 SNP309, and A2164G may collectively be associated with non-oropharyngeal cancer susceptibility and that MDM2 SNP309 in combination with TP53 codon 72 may accelerate the development of non-oropharyngeal cancer in women. A 2011 study found that TP53 codon 72 polymorphism was associated with an increased risk of lung cancer.

Meta-analyses from 2011 found no significant associations between TP53 codon 72 polymorphisms and both colorectal cancer risk and endometrial cancer risk. A 2011 study of a Brazilian birth cohort found an association between the non-mutant arginine TP53 and individuals without a family history of cancer. Another 2011 study found that the p53 homozygous (Pro/Pro) genotype was associated with a significantly increased risk for renal cell carcinoma.

MUC1. Mucin 1, cell surface associated (MUC1), also called polymorphic epithelial mucin (PEM) or epithelial membrane antigen or EMA, is a mucin encoded by the MUC1 gene in humans. MUC1 is a glycoprotein with extensive O-linked glycosylation of its extracellular domain. Mucins line the apical surface of epithelial cells in the lungs, stomach, intestines, eyes and several other organs. Mucins protect the body from infection by pathogen binding to oligosaccharides in the extracellular domain, preventing the pathogen from reaching the cell surface. Overexpression of MUC1 is often associated with colon, breast, ovarian, lung and pancreatic cancers. Joyce Taylor-Papadimitriou identified and characterised the antigen during her work with breast and ovarian tumors. MUC1 is a member of the mucin family and encodes a membrane bound, glycosylated phosphoprotein. MUC1 has a core protein mass of 120-225 kDa which increases to 250-500 kDa with glycosylation. It extends 200-500 nm beyond the surface of the cell.

The protein is anchored to the apical surface of many epithelia by a transmembrane domain. Beyond the transmembrane domain is a SEA domain that contains a cleavage site for release of the large extracellular domain. The release of mucins is performed by sheddases. The extracellular domain includes a 20 amino acid variable number tandem repeat (VNTR) domain, with the number of repeats varying from 20 to 120 in different individuals. These repeats are rich in serine, threonine and proline residues which permits heavy o-glycosylation.

Multiple alternatively spliced transcript variants that encode different isoforms of this gene have been reported, but the full-length nature of only some has been determined.

MUC1 is cleaved in the endoplasmic reticulum into two pieces, the cytoplasmic tail including the transmembrane domain and the extracellular domain. These domains tightly associate in a non-covalent fashion. This tight, non-covalent association is not broken by treatment with urea, low pH, high salt or boiling. Treatment with sodium dodecyl sulfate triggers dissociation of the subunits. The cytoplasmic tail of MUC1 is 72 amino acids long and contains several phosphorylation sites.

The protein serves a protective function by binding to pathogens and also functions in a cell signaling capacity.

Overexpression, aberrant intracellular localization, and changes in glycosylation of this protein have been associated with carcinomas. e.g., the CanAg tumor antigen is a novel glycoform of MUC1. In the cell nucleus, the protein MUC1 regulates the activity of transcription factor complexes that have a documented role in tumor-induced changes of host immunity.

PSMA. Prostate-specific membrane antigen (PSMA), glutamate carboxypeptidase II (GCPII), also known as N-acetyl-L-aspartyl-L-glutamate peptidase I (NAALADase I) or NAAG peptidase, is an enzyme that in humans is encoded by the FOLH1 (folate hydrolase 1) gene. Human PSMA contains 750 amino acids and weighs approximately 84 kDa. PSMA is a zinc metalloenzyme that resides in membranes. Most of the enzyme resides in the extracellular space. PSMA is a class II membrane glycoprotein. It catalyzes the hydrolysis of N-acetylaspartylglutamate (NAAG) to glutamate and N-acetylaspartate (NAA) according to the reaction scheme to the right.

Neuroscientists primarily use the term NAALADase in their studies, while those studying folate metabolism use folate hydrolase, and those studying prostate cancer or oncology, PSMA, all of which refer to the same protein.

PSMA is mainly expressed in four tissues of the body, including prostate epithelium, the proximal tubules of the kidney, the jejunal brush border of the small intestine and ganglia of the nervous system.

Indeed, the initial cloning of the cDNA encoding the gene expressing PSMA was accomplished with RNA from a prostate tumor cell line, LNCaP. PSMA shares homology with the transferrin receptor and undergoes endocytosis but the ligand for inducing internalization has not been identified. It was found that PSMA was the same as the membrane protein in the small intestine responsible for removal of gamma-linked glutamates from polygammaglutamate folate. This enables the freeing of folic acid, which then can be transported into the body for use as a vitamin. This resulted in the cloned genomic designation of PSMA as FOLH1 for folate hydrolase.

The three domains of the extracellular portion of PSMA—the protease, apical and C-terminal domains—collaborate in substrate recognition. The protease domain is a central seven-stranded mixed β-sheet. The β-sheet is flanked by 10 α-helices. The apical domain is located between the first and the second strands of the central β-sheet of the protease domain. The apical domain creates a pocket that facilitates substrate binding. The C-terminal domain is an Up-Down-Up-Down four-helix bundle.

The central pocket is approximately 2 nanometers in depth and opens from the extracellular space to the active site. This active site contains two zinc ions. During inhibition, each acts as a ligand to an oxygen in 2-PMPA or phosphate. There is also one calcium ion coordinated in PSMA, far from the active site. It has been proposed that calcium holds together the protease and apical domains. In addition, human PSMA has ten sites of potential glycosylation, and many of these sites (including some far from the catalytic domain) affect the ability of PSMA to hydrolyze NAAG.

The FOLH1 gene has multiple potential start sites and splice forms, giving rise to differences in membrane protein structure, localization, and carboxypeptidase activity based on the parent tissue.

Human PSMA is highly expressed in the prostate, roughly a hundred times greater than in most other tissues. In some prostate cancers, PSMA is the second-most upregulated gene product, with an 8- to 12-fold increase over levels in noncancerous prostate cells. Because of this high expression, PSMA is being developed as potential biomarker for therapy and imaging of some cancers. In human prostate cancer, the higher expressing tumors are associated with quicker time to progression and a greater percentage of patients suffering relapse. In vitro studies using prostate and breast cancer cell lines with decreased PSMA levels showed a significant decrease in the proliferation, migration, invasion, adhesion and survival of the cells.

PSMA is the target of several nuclear medicine imaging agents for prostate cancer. Capromabpentide (marketed as PROSTASCINT) is bound to indium-111 for detection by a gamma camera. Second-generation antibodies and low-molecular-weight ligands for imaging and therapy are under development. PSMA can also be used experimentally to target treatment. Lutetium-177 is a beta emitter, bound to PSMA to deliver treat prostate tumors. In addition to the human prostate and prostate cancer, PSMA is highly expressed in tumor neovasculature, but not corresponding normal vasculature of all types of solid tumors including the kidney, breast and colon.

mRAS. Ras-related protein M-Ras, also known as muscle RAS oncogene homolog and R-Ras3, is a protein that in humans is encoded by the MRAS gene on chromosome 3. It is ubiquitously expressed in many tissues and cell types. This protein functions as a signal transducer for a wide variety of signaling pathways, including those promoting neural and bone formation as well as tumor growth. The MRAS gene also contains one of 27 SNPs associated with increased risk of coronary artery disease.

The MRAS gene resides on chromosome 3 at the band 3q22.3 and includes 10 exons. This gene produces 2 isoforms through alternative splicing. M-Ras is a member of the small GTPase superfamily under the Ras family, which also includes Rap1, Rap2, R-Ras, and R-Ras2 (TC21). This protein spans a length of 209 residues. Its N-terminal amino acid sequence shares 60-75% identity with that in the Ras protein while its effector region is identical with that in Ras. M-Ras shares a similar structure with H-Ras and Rap2A with the exception of its switch 1 conformation when bound to guanosine 5′-(beta,gamma-imido)triphosphate (Gpp(NH)p). Of the two states M-Ras can switch between, M-Ras is predominantly found in its state 1 conformation, which does not bind Ras effectors.

The MRAS gene is expressed specifically in brain, heart, myoblasts, myotubes, fibroblasts, skeletal muscles, and uterus, suggesting a specific role of M-Ras in these tissue and cells. M-Ras is involved in many biological processes by activating a wide variety of proteins. For instance, it is activated by Ras guanine nucleotide exchange factors and can bind/activate some Ras protein effectors. M-Ras also weakly stimulates the mitogen-activated protein kinase (MAPK) activity and ERK2 activity, but modestly stimulates trans-activation from different nuclear response elements which bind transcription factors, such as SRF, ETS/TCF, Jun/Fos, and NF-kB/Rel. M-Ras has been found to induce Akt kinase activity in the PI3-K pathway, and it may play a role in cell survival of neural-derived cells. Moreover, M-Ras plays a crucial role in the downregulation of OCT4 and NANOG protein levels upon differentiation and has been demonstrated to modulate cell fate at early steps of development during neurogenesis. M-Ras, induced and activated by BMP-2 signaling, also participates in the osteoblastic determination, differentiation, and transdifferentiation under p38 MAPK and JNK regulation. M-Ras is involved in TNF-α-stimulated and Rap1-mediated LFA-1 activation in splenocytes. More generally, cells transfected with M-Ras exhibit dendritic appearances with microspikes, suggesting that M-Ras may participate in reorganization of the actin cytoskeleton. In addition, it is reported that M-Ras forms a complex with SCRIB and SHOC2, a polarity protein with tumor suppressor properties, and may play a key role in tumorigenic growth.

S100P. S100 calcium-binding protein P (S100P) is a protein that in humans is encoded by the S100P gene. The protein encoded by this gene is a member of the S100 family of proteins containing 2 EF-hand calcium-binding motifs. S100 proteins are localized in the cytoplasm and/or nucleus of a wide range of cells and involved in the regulation of a number of cellular processes such as cell cycle progression and differentiation. S100 genes include at least 13 members which are located as a cluster on chromosome 1q21; however, this gene is located at 4p16. This protein, in addition to binding Ca²⁺, also binds Zn²⁺ and Mg²⁺. This protein may play a role in the etiology of prostate cancer. S100P has been shown to interact with EZR and RAGE. The interactions between S100P and RAGE are disrupted by cromolyn and pentamidine.

### III. Therapeutic Administration

In another aspect, the present disclosure provides methods of inhibiting the growth or promoting the killing of a tumor cell or treating cancer, such as melanoma, by administering a recombinant oncolytic virus according to the instant disclosure at a multiplicity of infection sufficient to inhibit the growth of a tumor cell or to kill a tumor cell. In certain embodiments, the recombinant oncolytic virus is administered more than once, preferably twice, three times, or up to 10 times.

Examples of tumor cells or cancers that may be treated using the methods of this disclosure include breast cancer, ovarian cancer, renal cell carcinoma (RCC), melanoma (e.g., metastatic malignant melanoma), prostate cancer, colon cancer, lung cancer (including small cell lung cancer and non-small cell lung cancer), bone cancer, osteosarcoma, rhabdomyosarcoma, leiomyosarcoma, chondrosarcoma, pancreatic cancer, skin cancer, fibrosarcoma, chronic or acute leukemias including acute lymphocytic leukemia (ALL), adult T-cell leukemia (T-ALL), acute myeloid leukemia, chronic myeloid leukemia, acute lymphoblastic leukemia, chronic lymphocytic leukemia, lymphangiosarcoma, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), Castleman's disease, Kaposi's Sarcoma, hemangiosarcoma, multiple myeloma, Waldenstrom's macroglobulinemia and other B-cell lymphomas, nasopharangeal carcinomas, head or neck cancer, myxosarcoma, liposarcoma, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, cervical carcinoma, vaginal carcinoma, vulvar carcinoma, transitional cell carcinoma, esophageal cancer, malignant gastrinoma, small intestine cancer, cholangiocellular carcinoma, adenocarcinoma, endocrine system cancer, thyroid gland cancer, parathyroid gland cancer, adrenal gland cancer, sarcoma of soft tissue, urethral, penile cancer, testicular cancer, malignant teratoma, solid tumors of childhood, bladder cancer, kidney or ureter cancer, carcinoma of the renal pelvis, malignant meningioma, neoplasm of the central nervous system (CNS), tumor angiogenesis, spinal axis tumor, pituitary adenoma, epidermoid cancer, squamous cell cancer, environmentally induced cancers including those induced by asbestos, e.g., mesothelioma, and combinations of these cancers. Many cancers overexpress immune checkpoint proteins, such as PDL1 (PDL1⁺). The methods of this disclosure may be used to treat tumors or cancers regardless of PDL1 status.

Oncolytic viruses according to the disclosure may be administered locally or systemically. For example, without limitation, oncolytic viruses according to the disclosure can be administered intravascularly (intraarterially or intravenously), intratumorally, intramuscularly, intradermally, intraperitoneally, subcutaneously, orally, parenterally, intranasally, intratracheally, percutaneously, intraspinally, ocularly, or intracranially.

In still another embodiment, the methods involve parenteral administration of a recombinant oncolytic virus, preferably via an artery or via an in-dwelling medical device. The recombinant oncolytic virus can be administered with an immunotherapeutic agent or immunomodulator, such as an antibody that binds to a tumor-specific antigen (e.g., chimeric, humanized or human monoclonal antibodies). In another embodiment, the recombinant oncolytic virus treatment may be combined with surgery (e.g., tumor excision), radiation therapy, chemotherapy, or immunotherapy, and can be administered before, during or after a complementary treatment.

In other embodiments, the method involves ex vivo transduction of cells with a myxoma virus of the present invention, followed by administration of a composition comprising the cells into a subject. In certain embodiments, the cells may be autologous, i.e., the subject's own cells. In autologous embodiments, the cells may be obtained from the subject, transduced with a myxoma virus of the present invention, and re-administered into the subject, in a process similar to apheresis. Exemplary formulations for ex vivo delivery of the virus into cells may include the use of various transduction agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In certain embodiments, the recombinant oncolytic virus and an immunotherapeutic agent or immunomodulator can be administered concurrently or sequentially in a way that the agent does not interfere with the activity of the virus. In certain embodiments, the recombinant oncolytic virus is administered intra-arterially, intravenously, intraperitoneally, intratumorally, or any combination thereof. In still another embodiment, an interferon, such as interferon-α or pegylated interferon, is administered prior to administering the recombinant oncolytic virus according to the instant disclosure.

Oncolytic viruses according to the disclosure may be administered in a single administration or multiple administrations. The virus may be administered at dosage of 1×10⁵ plaque forming units (PFU), 5×10⁵ PFU, at least 1×10⁶ PFU, 5×10⁶ or about 5×10⁶ PFU, 1×10⁷, at least 1×10⁷ PFU, 1×10⁸ or about 1×10⁸ PFU, at least 1×10⁸ PFU, about or at least 5×10⁸ PFU, 1×10⁹ or at least 1×10⁹ PFU, 5×10⁹ or at least 5×10⁹ PFU, 1×10¹⁰ PFU or at least 1×10¹⁰ PFU, 5×10¹⁰ or at least 5×10¹⁰ PFU, 1×10¹¹ or at least 1×10¹¹, 1×10¹² or at least 1×10¹², 1×10¹³ or at least 1×10¹³. For example, the virus may be administered at a dosage of between about 10⁷-10¹³, between about 10⁸-10¹³, between about 10⁹-10¹², or between about 10⁸-10¹².

A. Combination Therapies

Additional therapies may be combined with any of the methods of the disclosure heretofore described in order to increase the killing of cancer cells, the inhibition of cancer cell growth, the inhibition of angiogenesis or otherwise improve the reverse or reduction of malignant phenotype of tumor cells. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the oncolytic virus and the other includes a second agent therapy.

Alternatively, the treatment may precede or follow the other agent or treatment by intervals ranging from minutes to weeks. In embodiments where the agents are applied separately to the cell, one would generally ensure that a significant period of time did not expire between each delivery, such that the agents would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) to several months (1, 2, 3, 4, 5, 6, 7 or 8) lapse between the respective administrations.

It also is conceivable that more than one administration of either agent will be desired. Various combinations may be employed, e.g. where one or more oncolytic virus treatment is administered before the administration of a second agent; or the second agent may be administered prior to oncolytic virus administration. Successive administration can include one or more administration of the oncolytic virus therapy or second agent. Again, to achieve cell killing, both agents are delivered to a cell in a combined amount effective to kill the cell. For example, the combination of the claimed PD1-expressing constructs and an immune modulator.

In accordance with certain embodiments of the present disclosure, methods for treating cancer are provided that can be used in conjunction with oncolytic virus therapy once a subject is identified as a responder or likely to respond to such therapy (e.g. vMYX-PD1 therapy). Such therapies may be utilized when the assays of the present disclosure indicate that a subject is unlikely to respond to treatment with a replication competent oncolytic virus such as myxoma virus. Alternatively, such therapies may be utilized in combination with replication competent oncolytic virus such as adenovirus in the case that a subject is identified by the present methods as unlikely to respond to treatment with only replication competent oncolytic virus.

Approximately 60% of persons with cancer will undergo surgery of some type, which includes preventative, diagnostic, staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

In certain aspects, a therapy is administered by intratumoral injection prior to surgery or upon excision of a part of or all of cancerous cells, tissue or tumor. Treatment may also be accomplished by perfusion, direct injection or local application of these areas with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages.

A wide variety of chemotherapeutic agents may be used in accordance with the present disclosure. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall within the following categories: alkylating agents, antimetabolites, antitumor antibiotics, topoisomerase inhibitors, and mitotic inhibitors.

Alkylating agents direct interact with genomic DNA to prevent the cancer cell from proliferating. This category of drugs includes agents that affect all phases of the cell cycle and are commonly used to treat chronic leukemia, non-Hodgkin's lymphoma, Hodgkin's disease, malignant melanoma, multiple myeloma, and particular cancers of the breast, lung, and ovary. They include nitrogen mustards such as mechlorethamine (nitrogen mustard), chlorambucil, cyclophosphamide (Cytoxan®), ifosfamide and melphalan, nitrosoureas such as streptozocin, carmustine (BCNU) and lomustine, alkyl sulfonates such as busulfan, triazines such as dacarbazine (DTIC) and temozolomide (Temodar), ethylenimines such as thiotepa and altretamine (hexamethylmelamine), and platinum drugs such as cisplatin, carboplatin, and oxalaplatin.

Antimetabolites disrupt DNA and RNA synthesis. Unlike alkylating agents, they specifically influence the cell cycle during S phase. They have been used to combat chronic leukemias, and tumors of the breast, ovary and gastrointestinal tract. Antimetabolites include 5-fluorouracil (5-FU), 6-mercaptopurine (6-MP), capecitabine (Xeloda®), cladribine, clofarabine, cytarabine (Ara-C®), floxuridine, fludarabine, gemcitabine (Gemzar®), hydroxyurea, methotrexate, pemetrexed, pentostatin and thioguanine.

Antitumor antibiotics have both antimicrobial and cytotoxic activity. These drugs also interfere with DNA by chemically inhibiting enzymes and mitosis or altering cellular membranes. These agents work in all phases of the cell cycle and are used to treat a variety of cancers. Representative examples include daunorubicin, doxorubicin (Adriamycin®), epirubicin, idarubicin, actinomycin-D, bleomycin and mitomycin-C. Generally, these compounds are administered by bolus i.v. injections at doses ranging from 25-100 mg/kg

Topoisomerase inhibitors interfere with topoisomerases, enzymes which help separate DNA strands so they can be copied and are used to treat certain leukemias, as well as lung, ovarian, gastrointestinal and other cancers and include topotecan, irinotecan, etoposide (VP-16) and teniposide.

Mitotic inhibitors, often plant alkaloids, work during M phase of the cell cycle and prevent mitosis or inhibit enzymes from producing proteins required for cell reproduction. Representative examples include taxanes such as paclitaxel (Taxol®) and docetaxel (Taxotere®), epothilones such as ixabepilone (Ixempra®), vinca alkaloids such as vinblastine (Velban®), vincristine (Oncovin®) and vinorelbine (Navelbine®), and Estramustine (Emcyt®).

In some embodiments, immunotherapy may be treatment with an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA).

The immune checkpoint inhibitors may be drugs such as small molecules, recombinant forms of ligand or receptors, or, in particular, are antibodies, such as human antibodies directed to the immune checkpoint proteins (e.g., International Patent Publication WO2015016718; Pardon, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab. Exemplary immune checkpoint inhibitors include PD-1 inhibitors, such as Pembrolizumab and Nivolumab; PD-L1 inhibitors, such as Atezolizumab, Avelumab, and Durvalumab; and CTLA-4 inhibitors, such as Ipilimumab.

In certain preferred embodiments, additive anti-tumor effects can be achieved by combining myxoma vPD1 with blockade of PD1 on T-cells directly. Clinically, this may be achieved through the use of antibodies that bind PD1 blocking interaction with PDL1. It is observed clinically that combination of individual immune checkpoint inhibition achieves much better antitumor activity (Johnson and Win, 2017, incorporated herein by reference in its entirety). An additional benefit of combing myxoma vPD1 and anti-PD1 antibodies according to the present invention may be in the setting of metastatic disease. Locally administered myxoma vPD1, via intra-tumoral injection, may not be optimal for metastatic disease due to PK/PD issues.

While combining the oncolytic viruses according to the present invention with anti-PD1 antibodies is a promising approach, possible complications might occur through interaction of the anti-PD1 antibody and soluble PD1 expressed from the myxoma virus. To ameliorate this possibility, a myxoma virus was produced expressing a PD1 construct containing mutations in the CD loop that prevents antibody recognition between the two clinically approved anti-PD1 antibodies. In one embodiment, a site mutation at position D85G in the PD1 protein will completely abolish the binding of anti-PD1 antibody pembrolizumab to PD1 (Tan et al., 2017, incorporated herein by reference in its entirety; and Na et al., incorporated herein by reference in its entirety). Thus, in this embodiment, introducing a single point mutation or combinations of single point mutations between the CD loop in the truncated PD1 myxoma construct will decrease any inhibitory binding of anti-PD1 antibody.

Other chemotherapeutic agents include targeted therapies such as imatinib (Gleevec®), gefitinib (Iressa®), sunitinib (Sutent®), sorafenib (Nexavar®), bortezomib (Velcade®), bevacizumab (Avastin®), trastuzumab (Herceptin®), cetuximab (Erbitux®), and panitumumab (Vectibix®), hormone therapies including antiestrogens such as fulvestrant (Faslodex®), tamoxifen, toremifine, aromatase inhibitors such as anastrozole, exemstane and letrozole, progestins such as megestrol acetate, and gonadotropin-releasing hormone and immunotherapies such as antibodies against tumor specific antigens (e.g. prostate specific antigen, carcinoembryonic antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155) which may be conjugated to a drug or toxin (e.g. radionuclide, ricin A chain, cholera toxin, pertussis toxin).

Radiotherapy, also called radiation therapy, is the treatment of cancer and other diseases with ionizing radiation which may be used to treat localized solid tumors such as cancers of the skin, tongue, larynx, brain, breast or cervix, or may be used to treat cancers of the blood-forming cells (leukemia) and lymphatic system (lymphoma). Radiation therapy includes, without limitation, the use of y-rays, X-rays and/or the directed delivery of radioisotopes to tumor cells. Other forms of DNA damaging factors are contemplated such as microwaves and UV-irradiation. Dosage ranges for X-rays range from daily doses of 50-200 roentgens for prolonged periods of time (3 to 4 weeks), to single doses of 2000-6000 roentgens.

Radiotherapy also comprises the use of radiolabeled antibodies to deliver doses of radiation directly to the cancer site (e.g. radioimmunotherapy, conformal radiotherapy), high resolution intensity modulated radiotherapy, and stereotactic radio-surgery. Stereotactic radio-surgery (gamma knife) for brain and other tumors employs precisely targeted beams of gamma radiotherapy from hundreds of different angles. Only one session, taking about 4-5 hours is required.

B. Pharmaceutical Compositions

The recombinant oncolytic virus described herein can be administered as a pharmaceutical or medicament formulated with a pharmaceutically acceptable carrier. Accordingly, the recombinant oncolytic virus may be used in the manufacture of a medicament or pharmaceutical composition. Pharmaceutical compositions of the disclosure may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, sodium citrate, and the like.

Alternately, therapeutic agents may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation may be in the form of a syrup, elixir, emulsion, or an aqueous or non-aqueous suspension. For rectal administration, the disclosure compounds may be combined with excipients such as cocoa butter, glycerin, gelatin, or polyethylene glycols and molded into a suppository.

Therapeutic agents may be formulated to include other medically useful drugs or biological agents. The therapeutic agents also may be administered in conjunction with the administration of other drugs or biological agents useful for the disease or condition to which the disclosure compounds are directed.

The biologic or pharmaceutical compositions of the present disclosure can be formulated to allow the recombinant oncolytic virus contained therein to be bioavailable upon administration of the composition to a subject. The level of recombinant oncolytic virus in serum, tumors, and other tissues after administration can be monitored by various well-established techniques, such as antibody-based assays (e.g., ELISA). In certain embodiments, recombinant oncolytic virus compositions are formulated for parenteral administration to a subject in need thereof (e.g., a subject having a tumor), such as a non-human animal or a human. Preferred routes of administration include intravenous, intra-arterial, subcutaneous, intratumoral, or intramuscular.

Proper formulation is dependent upon the route of administration chosen, as is known in the art. For example, systemic formulations are an embodiment that includes those designed for administration by injection, e.g., subcutaneous, intra-arterial, intravenous, intramuscular, intrathecal or intraperitoneal injection, as well as those designed for intratumoral, transdermal, transmucosal, oral, intranasal, or pulmonary administration. In one embodiment, the systemic or intratumoral formulation is sterile. In embodiments for injection, the recombinant oncolytic virus compositions of the instant disclosure may be formulated in aqueous solutions, or in physiologically compatible solutions or buffers such as Hanks's solution, Ringer's solution, mannitol solutions or physiological saline buffer. In certain embodiments, any of the recombinant oncolytic virus compositions described herein may contain formulator agents, such as suspending, stabilizing or dispersing agents. In embodiments for transmucosal administration, penetrants, solubilizers or emollients appropriate to the harrier to be permeated may be used in the formulation. For example, 1-dodecylhexahydro-2H-azepin-2-one (Azon®), oleic acid, propylene glycol, menthol, diethyleneglycol ethoxyglycol monoethyl ether (Transcutol®), polysorbate polyethylenesorbitan monolaurate (Tween®-20), and the drug 7-chloro-1-methyl-5-phenyl-3H-1,4-benzodiazepin-2-one (Diazepam), isopropyl myristate, and other such penetrants, solubilizers or emollients generally known in the art may be used in any of the compositions of the instant disclosure.

Administration can be achieved using a combination of routes, e.g., first administration using an intra-arterial route and subsequent administration via an intravenous or intratumoral route, or any combination thereof.

### IV. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Generation and Characterization of vMYX-PD1 Constructs

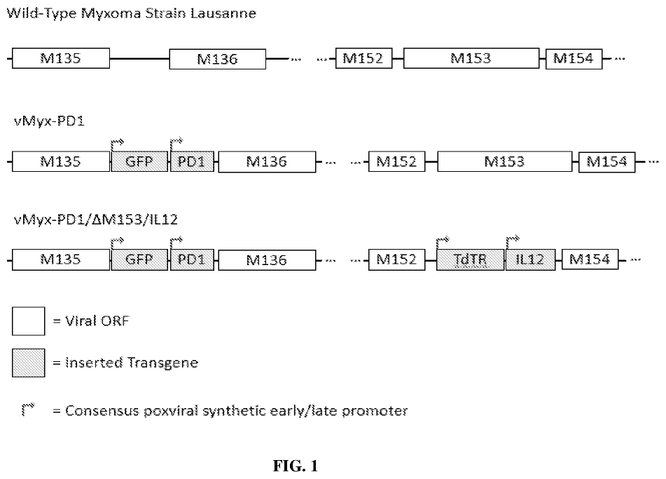

Recombinant virus construct may be made with soluble PD1 or soluble PD1 and optionally various cytokines/chemokines and/or tumor antigens. See, e.g., the schematics of a representative recombinant viral genomic structure in FIG. 1.

To construct the vPD1 and mutant vPD1, the extracellular region of human PD1 (amino acids 1-168) was amplified from a preconstructed template plasmid (PlasmID database, clone HsCD00345685) by PCR using the following primers.

Forward Primer:
[SEQ ID NO: 1]
ATCGCCCGGGAAAAATTGAAATTTTATTTTTTTTTTTTGGAATATAAATAACCATG
CAGATCCCACAGGCGCC
Reverse Primer:
[SEQ ID NO: 2]
ATCGGAATTCTCAGGTTTGGAACTGGCCGGCTG
Soluble PD1 nucleotide sequence:
[SEQ ID NO: 3]
ATGCAGATCCCACAGGCGCCCTGGCCAGTCGTCTGGGCGGTGCTACAACTGGGC
TGGCGGCCAGGATGGTTCTTAGACTCCCCAGACAGGCCCTGGAACCCCCCCACCT
TCTCCCCAGCCCTGCTCGTGGTGACCGAAGGGGACAACGCCACCTTCACCTGCAG
CTTCTCCAACACATCGGAGAGCTTCGTGCTAAACTGGTACCGCATGAGCCCCAGC
AACCAGACGGACAAGCTGGCCGCTTTCCCCGAGGACCGCAGCCAGCCCGGCCAG
GACTGCCGCTTCCGTGTCACACAACTGCCCAACGGGCGTGACTTCCACATGAGCG
TGGTCAGGGCCCGGCGCAATGACAGCGGCACCTACCTCTGTGGGGCCATCTCCCT
GGCCCCCAAGGCGCAGATCAAAGAGAGCCTGCGGGCAGAGCTCAGGGTGACAG
AGAGAAGGGCAGAAGTGCCCACAGCCCACCCCAGCCCCTCACCCAGGCCAGCCG
GCCAGTTCCAAACC
Native Soluble PD1 amino acid sequence (1-168)
Q15116 20 amino signal peptide
[SEQ ID NO: 4]
MQIPQAPWPV VWAVLQLGWR PGWFLDSPDR PWNPPTFSPA LLVVTEGDNA
TFTCSFSNTS ESFVLNWYRM SPSNQTDKLA AFPEDRSQPG QDCRFRVTQL
PNGRDFHMSV VRARRNDSGT YLCGAISLAP KAQIKESLRA ELRVTERRAE
VPTAHPSPSP RPAGQFQT
Mutated Soluble PD1 amino acid sequence (1-168) (D85G substitution
abolishes the binding of pembrolizumab to PD1)
20 amino signal peptide
[SEQ ID NO: 5]
MQIPQAPWPV VWAVLQLGWR PGWFLDSPDR PWNPPTFSPA LLVVTEGDNA
TFTCSFSNTS ESFVLNWYRM SPSNQTDKLA AFPEGRSQPG QDCRFRVTQL
PNGRDFHMSV VRARRNDSGT YLCGAISLAP KAQIKESLRA ELRVTERRAE
VPTAHPSPSP RPAGQFQT
Human IL-2 amino acid sequence (1-153)
P60568 20 amino signal peptide
[SEQ ID NO: 6]
MYRMQLLSCI ALSLALVTNS APTSSSTKKT QLQLEHLLLD LQMILNGINN
YKNPKLTRML TFKFYMPKKA TELKHLQCLE EELKPLEEVL NLAQSKNFHL
RPRDLISNIN VIVLELKGSE TTFMCEYADE TATIVEFLNR WITFCQSIIS TLT
Human IL-12, subunit alpha amino acid sequence (1-219)
P29459 22 amino signal peptide
[SEQ ID NO: 7]
MCPARSLLLV ATLVLLDHLS LARNLPVATP DPGMFPCLHH SQNLLRAVSN
MLQKARQTLE FYPCTSEEID HEDITKDKTS TVEACLPLEL TKNESCLNSR
ETSFITNGSC LASRKTSFMM ALCLSSIYED LKMYQVEFKT MNAKLLMDPK
RQIFLDQNML AVIDELMQAL NFNSETVPQK SSLEEPDFYK TKIKLCILLH
AFRIRAVTID RVMSYLNAS
Human IL-12, subunit beta amino acid sequence (1-328)
P29460 22 amino signal peptide
[SEQ ID NO: 8]
MCHQQLVISW FSLVFLASPL VA IWELKKDV YVVELDWYPD APGEMVVLTC
DTPEEDGITW TLDQSSEVLG SGKTLTIQVK EFGDAGQYTC HKGGEVLSHS
LLLLHKKEDG IWSTDILKDQ KEPKNKTFLR CEAKNYSGRF TCWWLTTIST
DLTFSVKSSR GSSDPQGVTC GAATLSAERV RGDNKEYEYS VECQEDSACP
AAEESLPIEV MVDAVHKLKY ENYTSSFFIR DIIKPDPPKN LQLKPLKNSR
QVEVSWEYPD TWSTPHSYFS LTFCVQVQGK SKREKKDRVF TDKTSATVIC
RKNASISVRA QDRYYSSSWS EWASVPCS
High Affinity Human IL-2 amino acid variant (1-164)
P60568 20 amino signal peptide
[SEQ ID NO: 9]
MYRMQLLSCI ALSLALVTNS APTSSSTKKT QLQLEHLLLD LQMILNGINN
YKNPKLTRML TFKFYMPKKA TELKHLQCLE EELKPLEEVL NLAQSKNFHF
DPRDVVSNIN VFVLELKGSE TTFMCEYADE TATIVEFLNR WITFCQSIIS
TLTAAAHHHH HHHH

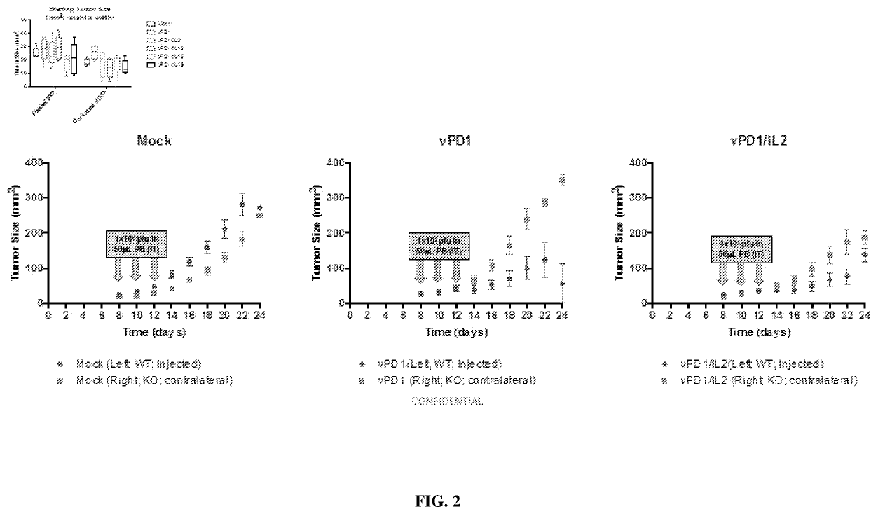

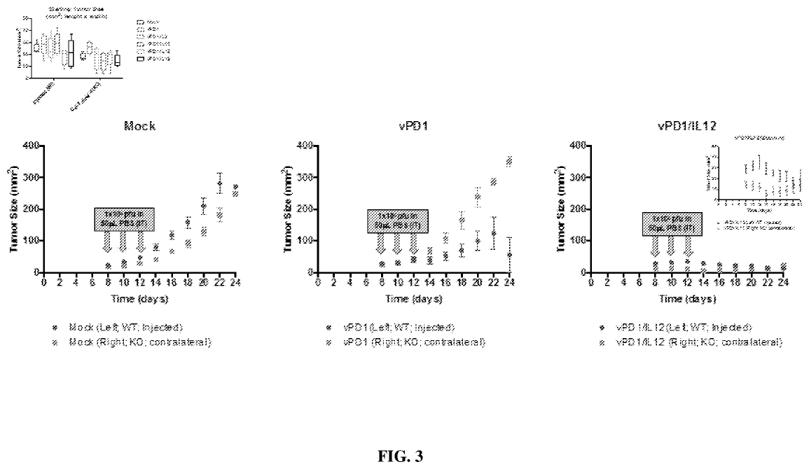

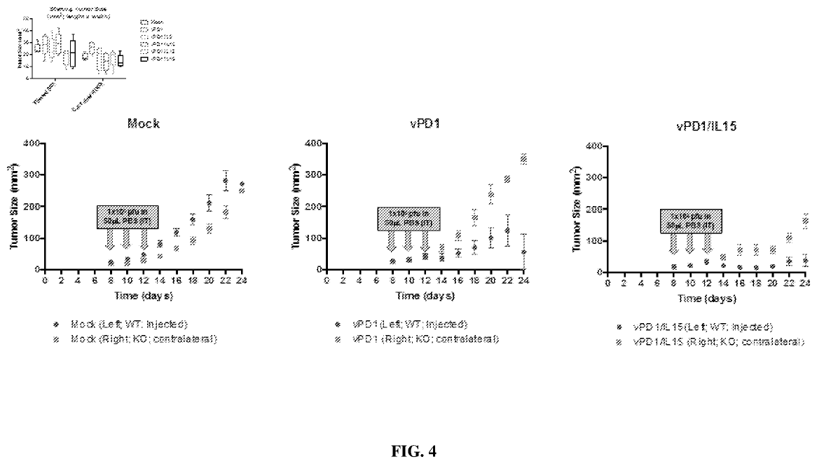

FIG. 2 shows a vPD1-IL2 efficacy study in subcutaneous B16F10 (B16F10 PD1L-KO) contralateral xenograft model. FIG. 3 shows a vPD1-IL12 efficacy study in subcutaneous B16F10 (B16F10 PD1L-KO) contralateral xenograft model. FIG. 4 shows the results of a vPD1-IL15 efficacy study in subcutaneous B16F10 (B16F10 PD1L-KO) contralateral xenograft model. Likewise, FIG. 5 shows results from a vPD1-IL18 efficacy study in subcutaneous B16F10 (B16F10 PD1L-KO) contralateral xenograft model. Taken together, IL-2 and IL-15 appear to show modest reductions in tumor size, while IL-12 provides the greatest reduction. IL-18 appears to have the least effect.

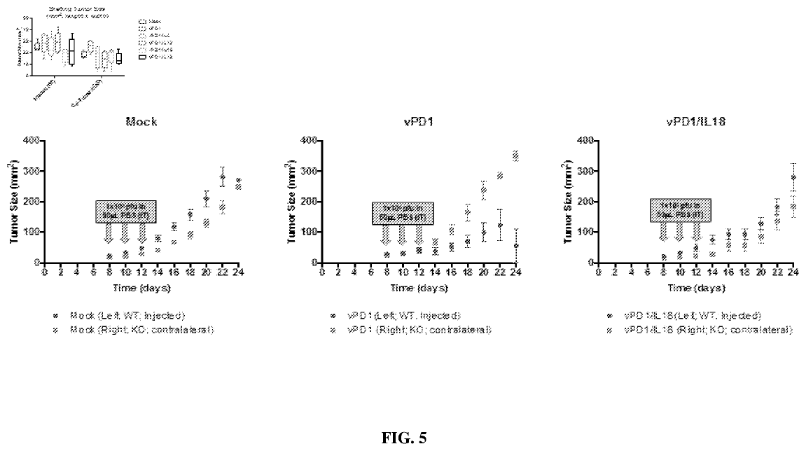

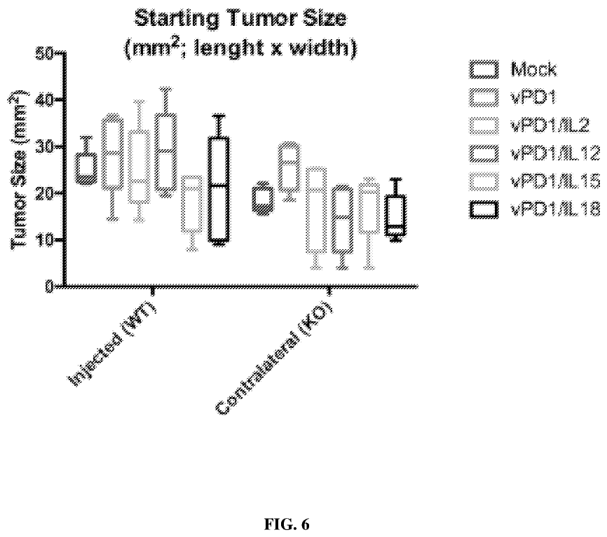

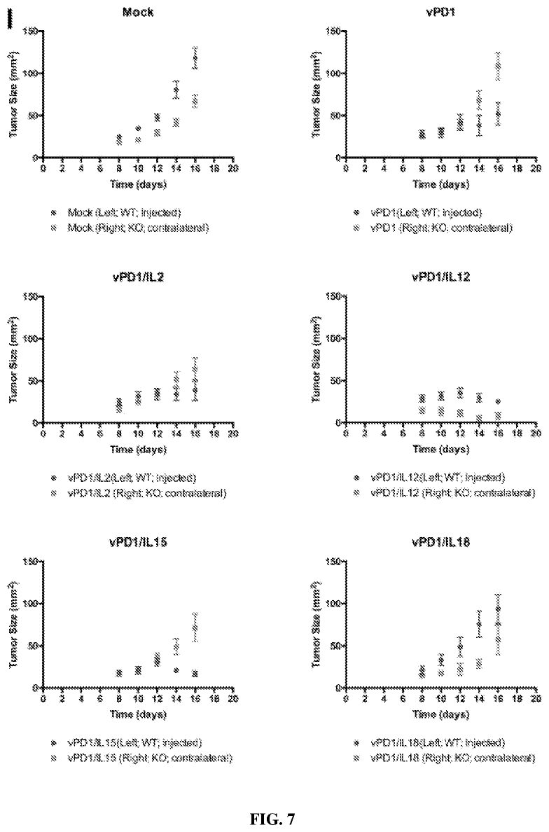

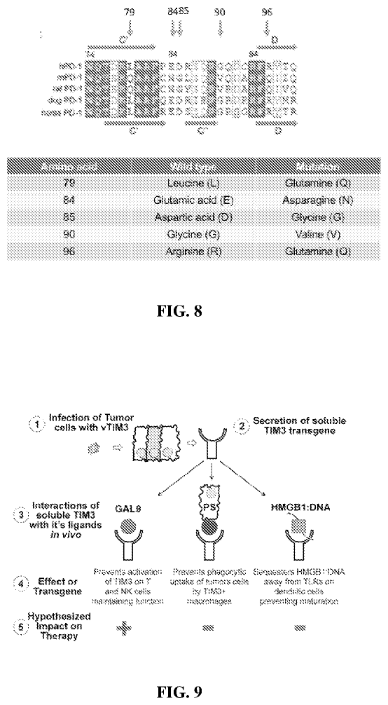

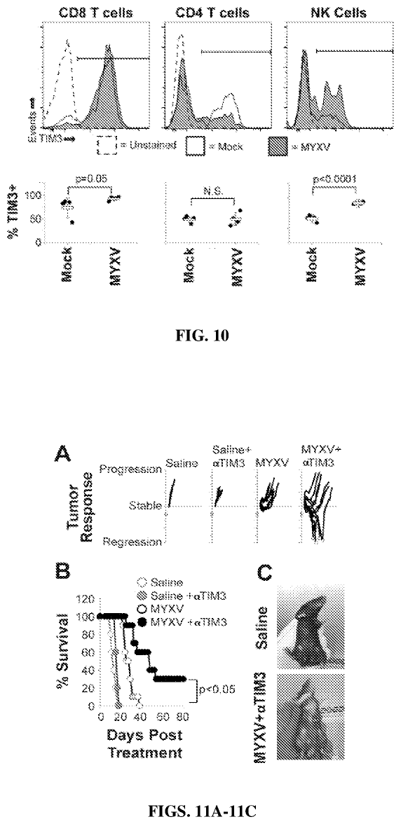

As shown in FIGS. 6 and 7, an in vivo subcutaneous (SC) contralateral mouse model was employed to test various viral constructs. Three intratumoral injections were made two days apart on the left side (WT-B16/F10) while the right side (PDL1-KO-B16/F10) was untreated. Injected tumors (left) and contralateral non-injected tumors (right) seem to respond to vPD1/IL12 and vPD1/IL2 treatments. vPD1/IL15 showed a modest response as well, whereas vPD/IL18 show negligible effect. Taken together, IL-12 surprisingly shows a greater reduction in the size of both the injected and the contralateral tumors than do the other constructs tested.

TABLE 1
RAW VIRUS TREATMENT DATA

| Treatment | | Animal | Admin | Day 8 | Day 10 | Day 12 | Day 14 | Day 16 |
|---|---|---|---|---|---|---|---|---|
| Mock | 1L | WT | Injected | 23.52 | 35.96 | 57.6 | 91.18 | 112.11 |
| Mock | 2L | WT | Injected | 22.96 | 32.83 | 43.56 | 113.46 | 151.51 |
| Mock | 3L | WT | Injected | 24.44 | 37.12 | 35.99 | 55.2 | 77.7 |
| Mock | 4L | WT | Injected | 22.05 | 26.95 | 49.64 | 69.3 | 123.42 |
| Mock | 5L | WT | Injected | 31.92 | 40.8 | 52.65 | 74.25 | 126.26 |
| Mock | 1R | KO | Contralateral | 17.22 | 16.92 | 29.15 | 45.14 | 85.36 |
| Mock | 2R | KO | Contralateral | 15.58 | 17.63 | 26.5 | 32.76 | 56.21 |
| Mock | 3R | KO | Contralateral | 20.16 | 19.68 | 21.12 | 31.92 | 57 |
| Mock | 4R | KO | Contralateral | 17.16 | 23 | 30.09 | 42.78 | 53.13 |
| Mock | 5R | KO | Contralateral | 22.05 | 28.05 | 42.48 | 56.7 | 83.66 |
| vPD1 | 1L | WT | Injected | 34.79 | 38.5 | 60.75 | 78.54 | 92.13 |
| vPD1 | 2L | WT | Injected | 14.43 | 11.22 | 11.22 | 6 | 11.55 |
| vPD1 | 3L | WT | Injected | 27.93 | 37.26 | 45.99 | 46.08 | 62.64 |
| vPD1 | 4L | WT | Injected | 28.62 | 34.22 | 58.855 | 33.6 | 55.68 |
| vPD1 | 5L | WT | Injected | 36.6 | 25.97 | 32.76 | 27.44 | 38.35 |
| vPD1 | 1R | KO | Contralateral | 22.95 | 23.5 | 36.58 | 63.75 | 105.06 |
| vPD1 | 2R | KO | Contralateral | 18.48 | 20.16 | 26.52 | 40.32 | 61.6 |
| vPD1 | 3R | KO | Contralateral | 26.52 | 35.4 | 31.8 | 51.84 | 86.49 |
| vPD1 | 4R | KO | Contralateral | 30.6 | 42.25 | 55.44 | 96.03 | 147.84 |
| vPD1 | 5R | KO | Contralateral | 29.7 | 36 | 51.12 | 90.25 | 141.52 |
| VPD1/IL2 | 1L | WT | Injected | 22 | 32.86 | 44.53 | 55.25 | 74.48 |
| VPD1/IL2 | 2L | WT | Injected | 26.5 | 38.86 | 37.8 | 21.5 | 17.22 |
| VPD1/IL2 | 3L | WT | Injected | 14.28 | 17.2 | 10.56 | 15.84 | 11.2 |
| VPD1/IL2 | 4L | WT | Injected | 22.5 | 23.52 | 34.1 | 39.2 | 49.14 |
| VPD1/IL2 | 5L | WT | Injected | 39.68 | 46.8 | 43.55 | 39.04 | 40.3 |
| VPD1/IL2 | 1R | KO | Contralateral | 25.3 | 29.5 | 48.84 | 66.36 | 88.35 |
| VPD1/IL2 | 2R | KO | Contralateral | 24.99 | 25.38 | 32.94 | 40.8 | 50.05 |
| VPD1/IL2 | 3R | KO | Contralateral | 10.88 | 30.09 | 41.58 | 68.06 | 94.05 |
| VPD1/IL2 | 4R | KO | Contralateral | 4 | 16.4 | 20.7 | 23.52 | 26.01 |
| VPD1/IL2 | 5R | KO | Contralateral | 20.7 | 20.68 | 36 | 61.2 | 63.18 |
| VPD1/IL12 | 1L | WT | Injected | 19.5 | 20.24 | 34.2 | 15.54 | 28.52 |
| VPD1/IL12 | 2L | WT | Injected | 29 | 29.76 | 23.97 | 36.54 | 22.88 |
| VPD1/IL12 | 3L | WT | Injected | 22.09 | 24.99 | 24.01 | 19.35 | 15.64 |
| VPD1/IL12 | 4L | WT | Injected | 31.27 | 44.1 | 54.72 | 39.53 | 30 |
| VPD1/IL12 | 5L | WT | Injected | 42.25 | 39.76 | 39.76 | 36.5 | 28.91 |
| VPD1/IL12 | 1R | KO | Contralateral | 4 | 0 | 14.26 | 0 | 0 |
| VPD1/IL12 | 2R | KO | Contralateral | 14.8 | 12.48 | 0 | 0 | 22.05 |
| VPD1/IL12 | 3R | KO | Contralateral | 20.4 | 22.96 | 14.35 | 0 | 0 |
| VPD1/IL12 | 4R | KO | Contralateral | 21.42 | 22.95 | 22.09 | 18.62 | 11.84 |
| VPD1/IL12 | 5R | KO | Contralateral | 10.85 | 7.56 | 7.54 | 7 | 6 |
| VPD1/IL15 | 1L | WT | Injected | 7.84 | 13.02 | 24.96 | 11.88 | 8.99 |
| VPD1/IL15 | 2L | WT | Injected | 21.12 | 21.56 | 33.06 | 19.8 | 14.04 |
| VPD1/IL15 | 3L | WT | Injected | 15.96 | 10.15 | 19.78 | 18.48 | 14 |
| VPD1/IL15 | 4L | WT | Injected | 23.46 | 25.5 | 46.8 | 28.6 | 23.22 |
| VPD1/IL15 | 5L | WT | Injected | 23.46 | 35.4 | 29.12 | 25.44 | 24.48 |
| VPD1/IL15 | 1R | KO | Contralateral | 4 | 9.8 | 18.33 | 23.5 | 40.26 |
| VPD1/IL15 | 2R | KO | Contralateral | 20.7 | 24.5 | 38.5 | 48.51 | 64.6 |
| VPD1/IL15 | 3R | KO | Contralateral | 20.24 | 19.27 | 39.65 | 48.3 | 63.99 |
| VPD1/IL15 | 4R | KO | Contralateral | 23 | 30.24 | 48.18 | 80.84 | 134.82 |
| VPD1/IL15 | 5R | KO | Contralateral | 19.27 | 27.5 | 38.4 | 43.55 | 53.25 |
| VPD1/IL18 | 1L | WT | Injected | 21.62 | 36.58 | 55.89 | 116 | |
| VPD1/IL18 | 2L | WT | Injected | 36.6 | 52.56 | 83.7 | 101.01 | 122.72 |
| VPD1/IL18 | 3L | WT | Injected | 26.95 | 39.04 | 56.07 | 80.64 | 123.12 |
| VPD1/IL18 | 4L | WT | Injected | 10.88 | 20.68 | 24.5 | 39.65 | 57.72 |
| VPD1/IL18 | 5L | WT | Injected | 9 | 16.81 | 24.08 | 40.87 | 71.2 |
| VPD1/IL18 | 1R | KO | Contralateral | 15.6 | 15.99 | 20.09 | 27.56 | |
| VPD1/IL18 | 2R | KO | Contralateral | 12.95 | 17.766 | 17.55 | 22.36 | 51.62 |
| VPD1/IL18 | 3R | KO | Contralateral | 12.24 | 12.58 | 10.56 | 18.92 | 33.63 |
| VPD1/IL18 | 4R | KO | Contralateral | 9.9 | 14.4 | 12.8 | 24.36 | 34.8 |
| VPD1/IL18 | 5R | KO | Contralateral | 22.95 | 27.54 | 50.37 | 49.56 | 108.81 |

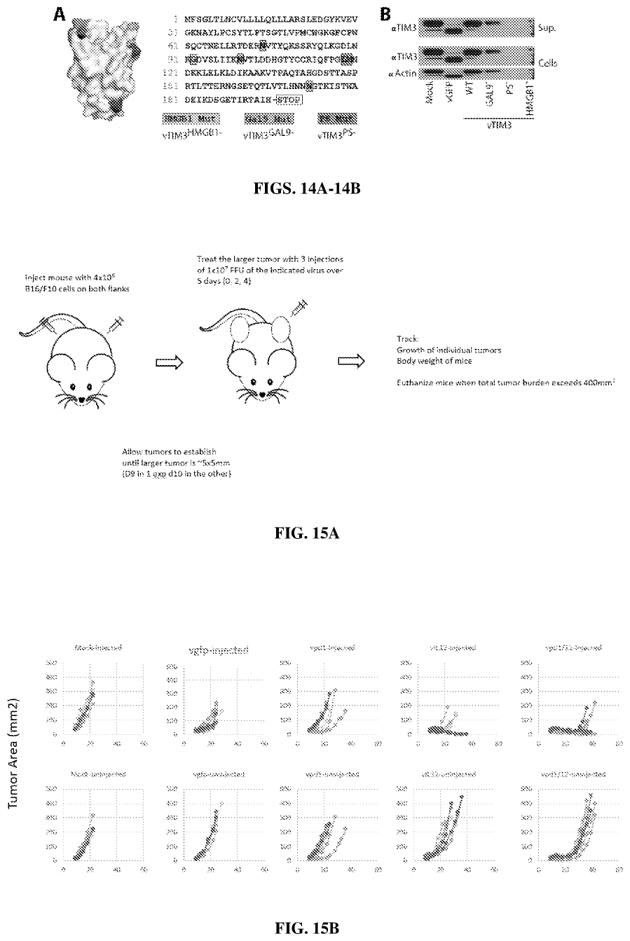

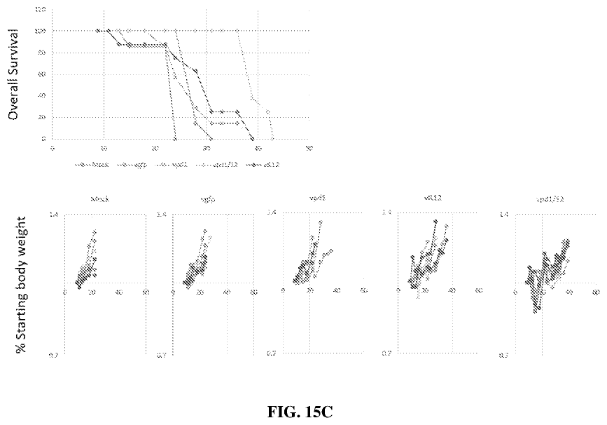

As shown in FIG. 15, a mouse study was performed to assess the efficacy of vPD1 alone, vIL12 alone, and the combination of vPD1+IL12. Mice were injected with 4×10⁶ B16/F10 cells on both flanks. After tumors were established, the larger tumor was treated with 3 injections of the indicated virus over 5 days (Day 0, 2, and 4). The growth of the tumors and the body weight of the mice were monitored until the mice were euthanized when the total tumor burden exceeded 400 mm². It was found that the combination of vPD1+IL12 had the most significant effect on decreasing tumor growth and increasing overall survival of the mice.

Further development of the virus comprised the addition of a transmembrane domain to the IL12. The transmembrane domain prevents the IL12 from leaking into the blood.

Transmembrane and cytosolic domain
(SEQ ID NO: 12)
CTTGTGCTCTTTGGGGCAGGATTCGGCGCAGTAATAACAGTCGTCGTCATC
GTTGTCATCATCAAATGCTTCTGTAAGCACAGAAGCTGTTTCAGAAGAAATGAGG
CAAGCAGAGAAACAAACAACAGCCTTACCTTCGGGCCTGAAGAAGCATTAGCTG
AACAGACCGTCTTCCTT
PD1-IL12-transmembrane domain construct
(SEQ ID NO: 13)
AGCGCCCAATACGCAAACCGCCTCTCCCCGCGCGTTGGCCGATTCATTAA
TGCAGCTGGCACGACAGGTTTCCCGACTGGAAAGCGGGCAGTGAGCGCAACGCA
ATTAATGTGAGTTAGCTCACTCATTAGGCACCCCAGGCTTTACACTTTATGCTTCC
GGCTCGTATGTTGTGTGGAATTGTGAGCGGATAACAATTTCACACAGGAAACAG
CTATGACCATGATTACGCCAAGCTCGAAATTAACCCTCACTAAAGGGAACAAAA
GCTGGAGCTCCACCGCGGTGGCGGCCGCATAAACGCGTTTAAACAGTCCCCCGT
ACGCGGTACATCGTACGCACACTTCACTAACGATGTCGTACATCGATTACACAAA
GAAGTAGAGTCATACGACGTACGTTTCCCTATAAAATCGGTAAACCTAGACGCG
GTGTTTCTATCCATAAACGTAACACGTGTACGTCTACGTTGGAAGATACCCTTGA
CCGAACACAATCCTTATCAGACGGCCTACGGATGTTCTAACGACAGATTATACAG
CTACAACGAGTACGCTTTTTCTCATTTAAAACAAGACCGTGTAAAGATCATAGAA
CTCCCATGTGACGACGATTACAGCGTCGTGTTAATCACACACGATAGCCGTTCGA
CTATTACACCGGATAAAGTGACCGGGTGGCTGCGCACGACCCGTCTACGTTACGT
AAACGTATCCCTACCCAAGGGTTCCACGGAAACGGGACACAACGTAACGTGTCT
AACTCCCACACACGTCAATCTATGTCATCGTTGTCGTATAACGATTACCAAAACG
GGCGTGGACGCAACCGCGTTCTCATGCGTCGACGGCGATACATGCACCGAACAC
GACACGACCGCGTCAACGTGTACGATTATTATAAAAACGACGGGACTGGACTTT
TTGTTTATGGGGAAACTCTAAAAAAAATTGTCAATTAAAGTAACTGCAGATCGAT
CGCATATGAAAATTGAAATTTTATTTTTTTTTTTTGGAATATAAATAATGGTGAGC
AAGGGCGAGGAGGTCATCAAAGAGTTCATGCGCTTCAAGGTGCGCATGGAGGGC
TCCATGAACGGCCACGAGTTCGAGATCGAGGGCGAGGGCGAGGGCCGCCCCTAC
GAGGGCACCCAGACCGCCAAGCTGAAGGTGACCAAGGGCGGCCCCCTGCCCTTC
GCCTGGGACATCCTGTCCCCCCAGTTCATGTACGGCTCCAAGGCGTACGTGAAGC
ACCCCGCCGACATCCCCGATTACAAGAAGCTGTCCTTCCCCGAGGGCTTCAAGTG
GGAGCGCGTGATGAACTTCGAGGACGGCGGTCTGGTGACCGTGACCCAGGACTC
CTCCCTCCAAGACGGCACGCTGATCTACAAGGTGAAGATGCGCGGCACCAACTT
CCCCCCCGACGGCCCCGTAATGCAGAAGAAGACCATGGGCTGGGAGGCCTCCAC
CGAGCGCCTGTACCCCCGCGACGGCGTGCTGAAGGGCGAGATCCACCAGGCCCT
GAAGCTGAAGGACGGCGGCCACTACCTGGTGGAGTTCAAGACCATCTACATGGC
CAAGAAGCCCGTGCAACTGCCCGGCTACTACTACGTGGACACCAAGCTGGACAT
CACCTCCCACAACGAGGACTACACCATCGTGGAACAGTACGAGCGCTCCGAGGG
CCGCCACCACCTGTTCCTGGGGCATGGCACCGGCAGCACCGGCAGCGGCAGCTC
CGGCACCGCCTCCTCCGAGGACAACAACATGGCCGTCATCAAAGAGTTCATGCG
CTTCAAGGTGCGCATGGAGGGCTCCATGAACGGCCACGAGTTCGAGATCGAGGG
CGAGGGCGAGGGCCGCCCCTACGAGGGCACCCAGACCGCCAAGCTGAAGGTGA
CCAAGGGCGGCCCCCTGCCCTTCGCCTGGGACATCCTGTCCCCCCAGTTCATGTA
CGGCTCCAAGGCGTACGTGAAGCACCCCGCCGACATCCCCGATTACAAGAAGCT
GTCCTTCCCCGAGGGCTTCAAGTGGGAGCGCGTGATGAACTTCGAGGACGGCGG
TCTGGTGACCGTGACCCAGGACTCCTCCCTCCAAGACGGCACGCTGATCTACAAG
GTGAAGATGCGCGGCACCAACTTCCCCCCCGACGGCCCCGTAATGCAGAAGAAG
ACCATGGGCTGGGAGGCCTCCACCGAGCGCCTGTACCCCCGCGACGGCGTGCTG
AAGGGCGAGATCCACCAGGCCCTGAAGCTGAAGGACGGCGGCCACTACCTGGTG
GAGTTCAAGACCATCTACATGGCCAAGAAGCCCGTGCAACTGCCCGGCTACTAC
TACGTGGACACCAAGCTGGACATCACCTCCCACAACGAGGACTACACCATCGTG
GAACAGTACGAGCGCTCCGAGGGCCGCCACCACCTGTTCCTGTACGGCATGGAC
GAGCTGTACAAGTAACCCGGGAAAAATTGAAATTTTATTTTTTTTTTTTGGAATAT
AAATAACCATGTGTCCTCAGAAGCTAACCATCTCCTGGTTTGCCATCGTTTTGCT
GGTGTCTCCACTCATGGCCATGTGGGAGCTGGAGAAAGACGTTTATGTTGTAGAG
GTGGACTGGACTCCCGATGCCCCTGGAGAAACAGTGAACCTCACCTGTGACACG
CCTGAAGAAGATGACATCACCTGGACCTCAGACCAGAGACATGGAGTCATAGGC
TCTGGAAAGACCCTGACCATCACTGTCAAAGAGTTTCTAGATGCTGGCCAGTACA
CCTGCCACAAAGGAGGCGAGACTCTGAGCCACTCACATCTGCTGCTCCACAAGA
AGGAAAATGGAATTTGGTCCACTGAAATTTTAAAAAATTTCAAAAACAAGACTTT
CCTGAAGTGTGAAGCACCAAATTACTCCGGACGGTTCACGTGCTCATGGCTGGTG
CAAAGAAACATGGACTTGAAGTTCAACATCAAGAGCAGTAGCAGTTCCCCTGAC
TCTCGGGCAGTGACATGTGGAATGGCGTCTCTGTCTGCAGAGAAGGTCACACTG
GACCAAAGGGACTATGAGAAGTATTCAGTGTCCTGCCAGGAGGATGTCACCTGC
CCAACTGCCGAGGAGACCCTGCCCATTGAACTGGCGTTGGAAGCACGGCAGCAG
AATAAATATGAGAACTACAGCACCAGCTTCTTCATCAGGGACATCATCAAACCA
GACCCGCCCAAGAACTTGCAGATGAAGCCTTTGAAGAACTCACAGGTGGAGGTC
AGCTGGGAGTACCCTGACTCCTGGAGCACTCCCCATTCCTACTTCTCCCTCAAGT
TCTTTGTTCGAATCCAGCGCAAGAAAGAAAAGATGAAGGAGACAGAGGAGGGG
TGTAACCAGAAAGGTGCGTTCCTCGTAGAGAAGACATCTACCGAAGTCCAATGC
AAAGGCGGGAATGTCTGCGTGCAAGCTCAGGATCGCTATTACAATTCCTCATGCA
GCAAGTGGGCATGTGTTCCCTGCAGGGTCCGATCCGGTGGCGGTGGCTCGGGCG
GTGGTGGGTCGGGTGGCGGCGGATCTAGGGTCATTCCAGTCTCTGGACCTGCCAG
GTGTCTTAGCCAGTCCCGAAACCTGCTGAAGACCACAGATGACATGGTGAAGAC
GGCCAGAGAAAAACTGAAACATTATTCCTGCACTGCTGAAGACATCGATCATGA
AGACATCACACGGGACCAAACCAGCACATTGAAGACCTGTTTACCACTGGAACT
ACACAAGAACGAGAGTTGCCTGGCTACTAGAGAGACTTCTTCCACAACAAGAGG
GAGCTGCCTGCCCCCACAGAAGACGTCTTTGATGATGACCCTGTGCCTTGGTAGC
ATCTATGAGGACTTGAAGATGTACCAGACAGAGTTCCAGGCCATCAACGCAGCA
CTTCAGAATCACAACCATCAGCAGATCATTCTAGACAAGGGCATGCTGGTGGCC
ATCGATGAGCTGATGCAGTCTCTGAATCATAATGGCGAGACTCTGCGCCAGAAA
CCTCCTGTGGGAGAAGCAGACCCTTACAGAGTGAAAATGAAGCTCTGCATCCTG
CTTCACGCCTTCAGCACCCGCGTCGTGACCATCAACAGGGTGATGGGCTATCTGA
GCTCCGCCCTTGTGCTCTTTGGGGCAGGATTCGGCGCAGTAATAACAGTCGTCGT
CATCGTTGTCATCATCAAATGCTTCTGTAAGCACAGAAGCTGTTTCAGAAGAAAT
GAGGCAAGCAGAGAAACAAACAACAGCCTTACCTTCGGGCCTGAAGAAGCATTA
GCTGAACAGACCGTCTTCCTTTGAGAATTCACGAATCGAATAAAAACCCGTGTAC
ACACGGACGTTAATTTTTTTTGTGGTTTAAAAAATGACCACATTTACGCTTTTTTT
TAACGCGTTATATAAGGTATCTCGTTTGTCTATAACAAAGATCGTAACTGACC
TTTTTTATATCGAGAAAACATACGTTTAGTTCATCCTCAAACGTAACACCGTAAC
TGCCTCGGACATCCTCCTTGTTGTCGTACACAAACATACTAATCGGATGCGTGAA
ATGAGGATTCACTTTAATCGGATTGGTTTCTAGGTTAACACATGTTACACAAGAT
CCTAAGATGGTTATGGACACATCCTTGTTGTGATGTAACGAGTCGGGAAGTTGAT
TGCCGTAGTTGCCCACGTCGCCCTCCGGTTCCAGACACGTAATGGTTAGGTATAT
ATCCGAATACTTCGTCAACGGATGAGTCGTAAATAACATGATGGATAGCTTGTTC
CCATCTCCTGCACCAGCACTGGCCGCCACAAATCGTTGTACCACGTTAGTAATCG
TAATGTTTATCATAAGCCCGTACCCGGTTAATATGAGCGTGGACGTTTTATGATC
GTATCGTTCCTTCATGTGACATTCTCCCATAACCGTTTCGACGTACCGATTTAACC
CGATGGTTAGCTCGGCGGCTAAGTGCCAGTGGATCCCCCAATTCGATATCAAGCT
TATCGATACCGTCGACCTCGAGGGGGGGCCCGGTACCCAATTCGCCCTATAGTGA
GTCGTATTACAATTCACTGGCCGTCGTTTTACAACGTCGTGACTGGGAAAACCCT
GGCGTTACCCAACTTAATCGCCTTGCAGCACATCCCCCTTTCGCCAGCTGGCGTA
ATAGCGAAGAGGCCCGCACCGATCGCCCTTCCCAACAGTTGCGCAGCCTGAATG
GCGAATGGGACGCGCCCTGTAGCGGCGCATTAAGCGCGGCGGGTGTGGTGGTTA
CGCGCAGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAAATCGGG
GGCTCCCTTTAGGGTTCCGATTTAGTGCTTTACGGCACCTCGACCCCAAAAAACT
TGATTAGGGTGATGGTTCACGTAGTGGGCCATCGCCCTGATAGACGGTTTTTCGC
CCTTTGACGTTGGAGTCCACGTTCTTTAATAGTGGACTCTTGTTCCAAACTGGAA
CAACACTCAACCCTATCTCGGTCTATTCTTTTGATTTATAAGGGATTTTGCCGATT
TCGGCCTATTGGTTAAAAAATGAGCTGATTTAACAAAAATTTAACGCGAATTTTA
ACAAAATATTAACGCTTACAATTTAGGTGGCACTTTTCGGGGAAATGTGCGCGGA
ACCCCTATTTGTTTATTTTTCTAAATACATTCAAATATGTATCCGCTCATGAGACA
ATAACCCTGATAAATGCTTCAATAATATTGAAAAAGGAAGAGTATGAGTATTCA
ACATTTCCGTGTCGCCCTTATTCCCTTTTTTGCGGCATTTTGCCTTCCTGTTTTTGC
TCACCCAGAAACGCTGGTGAAAGTAAAAGATGCTGAAGATCAGTTGGGTGCACG
AGTGGGTTACATCGAACTGGATCTCAACAGCGGTAAGATCCTTGAGAGTTTTCGC
CCCGAAGAACGTTTTCCAATGATGAGCACTTTTAAAGTTCTGCTATGTGGCGCGG
TATTATCCCGTATTGACGCCGGGCAAGAGCAACTCGGTCGCCGCATACACTATTC
TCAGAATGACTTGGTTGAGTACTCACCAGTCACAGAAAAGCATCTTACGGATGG
CATGACAGTAAGAGAATTATGCAGTGCTGCCATAACCATGAGTGATAAGACTGC
GGCCAACTTACTTCTGACAACGATCGGAGGACCGAAGGAGCTAACCGCTTTTTTG
CACAACATGGGGGATCATGTAACTCGCCTTGATCGTTGGGAACCGGAGCTGAAT
GAAGCCATACCAAACGACGAGCGTGACACCACGATGCCTGTAGCAATGGCAACA
ACGTTGCGCAAACTATTAACTGGCGAACTACTTACTCTAGCTTCCCGGCAACAAT
TAATAGACTGGATGGAGGCGGATAAAGTTGCAGGACCACTTCTGCGCTCGGCCC
TTCCGGCTGGCTGGTTTATTGCTGATAAATCTGGAGCCGGTGAGCGTGGGTCTCG
CGGTATCATTGCAGCACTGGGGCCAGATGGTAAGCCCTCCCGTATCGTAGTTATC
TACACGACGGGGAGTCAGGCAACTATGGATGAACGAAATAGACAGATCGCTGAG
ATAGGTGCCTCACTGATTAAGCATTGGTAACTGTCAGACCAAGTTTACTCATATA
TACTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAGC
GTCAGACCCCGTAGAAAAGATCAAAGGATCTTCTTGAGATCCTTTTTTTCTGCGC
GTAATCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGAGCGCA
GATACCAAATACTGTTCTTCTAGTGTAGCCGTAGTTAGGCCACCACTTCAAGAAC
TCTGTAGCACCGCCTACATACCTCGCTCTGCTAATCCTGTTACCAGTGGCTGCTGC
CAGTGGCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTTGGAG
CGAACGACCTACACCGAACTGAGATACCTACAGCGTGAGCTATGAGAAAGCGCC
ACGCTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAG
GGGGGCGGAGCCTATGGAAAAACGCCAGCAACGCGGCCTTTTTACGGTTCCTGG
CCTTTTGCTGGCCTTTTGCTCACATGTTCTTTCCTGCGTTATCCCCTGATTCTGTGG
ATAACCGTATTACCGCCTTTGAGTGAGCTGATACCGCTCGCCGCAGCCGAACGAC
CGAGCGCAGCGAGTCAGTGAGCGAGGAAGCGGAAG

### Example 2—Generation of vMYX-TIM3 Constructs

vTIM3 was generated by homologously recombining unmodified myxoma virus (strain Lausanne) with pBS-M135/M136-sE/L GFP+TIM3, a plasmid which contains the following critical elements:
pBluescript plasmid backbone
Region of myxoma genome homologous to M134/M135
eGFP driven by the consensus poxviral synthetic early/late promoter
aa1-195 of murine TIM3 driven by a second consensus poxviral synthetic early/late promoter
Region of myxoma genome homologous to M136

TIM3-GFP Construct
(SEQ ID NO: 10)
NNNNNNNNNNCNCGNGGNGGCGGCCGCTCTAGTAGGATTACCTGGTCTATATAG
ATAACAAAACCTACGTACGTATAAACGAGACCGTTGTACCGGAGAACGAGTATC
TGGCAGCGAAGGCCCCGCGAGTGACCTGTTTCCACACGGACTTGATCCCCATTAC
GGACGAAGAGACACAACGACGTTTTGAGAAAATGATTGTACAGGCGGCGTTAGA
GGACGCCCTAACGAGCATCTTTGAGGAGCACGACAATAACGTAACCGATTACTT
CGCGGAATACATGCGATCCCTCCAAATGGCGAATAAAAGTCATACGAATAATAT
TATCGCGGTCGCTTTAGCGGGGATAATCGTCATTGTAACGACCTACGTGTTTACT
AGATTACGCACTAAGCAAAAAAAAGGAAATTATAACGTACGTAATAAGATAGAT
AATTCCATACAGAAAGAGATTCAGTTGGACGGTGTATATACTACTGACAACGTTT
TTATATAAACATGGTGTTTATATTTATTATCACCTGTGTATGTTTGGTGACGAGAT
CCTGTGGGGGTGGGTTAGAAGACGATATAGATCGCATATTTCAAAAACGATACA
ACGAACTGAGCCAGCCGATTAANNNCAATATGCGTACACTGTGCAAGTTTAGAG
GAATTACCGCGACTATGTTTACGGAAGGAGAATCTTACCTTATTCAATGTCCCAT
AATTCACGATTACGTGCTACGGGCGCTGTATGACTTAGTGGAAGGAAGTTACAC
GGTACGCTGGGAACGCGAAACGGAAGACGATGTTGAGTCGGTAGATCCGAAGTT
AGTCAAAGGGACGCTATTATACCTCCAACCTAACGCGTCCAGTATAGGAACGTA
TCTATGTACCTTACACGATAACCGAGGTATGTGTTATCAATCTGTCGCGCACGTC
ATCCGACGTCCGAAGATGCAATGCGTGAAACATGCACATACGACATCGGACAGC
AACCTGTGGATATACCTCGCCATTTTAGCAGTTTTGATATCCTTAGGCGTCCTGTA
AAGGAAACGCGCCAGACTCCGGAACTATGAAGGATTTATCACTGTATACAGACT
CCGACGTACGAAGGATAATCACGACGTAACTCGAACTCTGCAGGTCGACTCTAG
AGGATCTACTAGTCATATGGATTTAAAAATAGCGGAGCTTAAAAATTGAAATTTT
ATTTTTTTTTTTTGGAATATAAATAAGCTCGAAGTCGACAGATCTAGGCCTGGTA
CCCGATCCACCGGTCGCCACCATGGTGAGCAAGGGCGAGGAGCTGTTCACCGGG
GTGGTGCCCATCCTGGTCGAGCTGGACGGCGACGTAAACGGCCACAAGTTCAGC
GTGTCCGGCGAGGGCGAGGGCGATGCCACNTACGGCAAGCTGACCCTGAAGTTC
ATCTGCACCACCGGCAAGCTGCCCGTGCCCTGGCCCACCCTCGTGACCACCCTGA
CCTACGGCGTGCAGTGCTTCAGCCGCTACCCCGACCACATGAAGCAGCACGACTT
CTTCAAGTCCGCCATGCCCGAAGGCTACGTCCAGGAGCGCACCATCTTCTTCAAG
GACGACGGCAACTACAAGACCCGCGCCGAGGTGAAGTTCGAGGGCGACACCCTG
GTGAACCGCATCGAGCTGAAGGGCATCGACTTCAAGGAGGACGGCAACATCCTG
GGGCACAAGCTGGAGTACAACTACAACAGCCACAACGTCTATATCATGGCCGAC
AAGCAGAAGAACGGCATCAAGGTGAACTTCAAGATCCGCCACAACATCGAGGAC
GGCAGCGTGCAGCTCGCCGACCACTACCAGCAGAACACCCCCATCGGCGACGGC
CCCGTGCTGNTGCCCGACAACCACTACCTGAGCACCCAGTCCGCCCTGAGCAAA
GACCCCAACGAGAAGCGCGATCACATGGTCCTGCTGGAGTTCGTGACCGCCGCC
GGGATCACTCTCGGCATGGACGAGCTGTACAAGTAAAGCGGCCGGGTAATTACC
CGGGATGTTTTCAGGTCTTACCCTCAACTGTGTCCTGCTGCTGCTGCAACTACTAC
TTGCAAGGTCATTGGAAAATGCTTATGTGTTTGAGGTTGGTAAGAATGCCTATCT
GCCCTGCAGTTACACTCTATCTACACCTGGGGCACTTGTGCCTATGTGCTGGGGC
AAGGGATTCTGTCCTTGGTCACAGTGTACCAACGAGTTGCTCAGAACTGATGAAA
GAAATGTGACATATCAGAAATCCAGCAGATACCAGCTAAAGGGCGATCTCAACA
AAGGAGACGTGTCTCTGATCATAAAGAATGTGACTCTGGATGACCATGGGACCT
ACTGCTGCAGGATACAGTTCCCTGGTCTTATGAATGATAAAAAATTAGAACTGAA
ATTAGACATCAAAGCAGCCAAGGTCACTCCAGCTCAGACTGCCCATGGGGACTC
TACTACAGCTTCTCCAAGAACCCTAACCACGGAGAGAAATGGTTCAGAGACACA
GACACTGGTGACCCTCCATAATAACAATGGAACAAAAATTTCCACATGGGCTGA
TGAAATTAAGGACTCTGGAGAAACGATCAGAACTGCTATCCACTAGGAATTCTA
ACATTTTTTAAAACAATTTCGTTATGTTAAATTATGGAACGGTCGCCCACTTACAC
GGTACACGATAAACGCTTTTCTATCGTCGCACTAAACGGACAATACGACATGGTG
GACGATTTTGGTCTTAGTTTTTCTTACACAGCGATCGACGATATTTCTAAAAATCA
TTCCATCAAACACGTTTTAGAAGAATACTTTTCATGGCGCGCGTATATAGGCCGG
GTATGTATCATACCGAATCACGTGGGAAAGCTCTACATCAAACTTACAAAGTTGG
ACACCACGGCGAAGAACAAACTAGGCAATCTAGATATATTGTTATGCGACGTGT
TAAAAATAGACGAGGACGGAGGCAACGAGAAACTGTTTCAATTCATACGGTCGC
GGATCCCCCAATTCGATATCAAGCTTATCGATACCGTCGACCTCG
Soluble TIM3
(SEQ ID NO: 11)
ATGTTTTCAGGTCTTACCCTCAACTGTGTCCTGCTGCTGCTGCAACTACTACTTGC
AAGGTCATTGGAAAATGCTTATGTGTTTGAGGTTGGTAAGAATGCCTATCTGCCC
TGCAGTTACACTCTATCTACACCTGGGGCACTTGTGCCTATGTGCTGGGGCAAGG
GATTCTGTCCTTGGTCACAGTGTACCAACGAGTTGCTCAGAACTGATGAAAGAAA
TGTGACATATCAGAAATCCAGCAGATACCAGCTAAAGGGCGATCTCAACAAAGG
AGACGTGTCTCTGATCATAAAGAATGTGACTCTGGATGACCATGGGACCTACTGC
TGCAGGATACAGTTCCCTGGTCTTATGAATGATAAAAAATTAGAACTGAAATTAG
ACATCAAAGCAGCCAAGGTCACTCCAGCTCAGACTGCCCATGGGGACTCTACTA
CAGCTTCTCCAAGAACCCTAACCACGGAGAGAAATGGTTCAGAGACACAGACAC
TGGTGACCCTCCATAATAACAATGGAACAAAAATTTCCACATGGGCTGATGAAA
TTAAGGACTCTGGAGAAACGATCAGAACTGCTATCCAC

pBS-M135/M136-sE/L GFP+TIM3 was transfected into BSC40 cells which were then infected with unmodified myxoma virus (strain Lausanne). Cells were cultured for 72 hours which produces recombinant viruses in which the untranslated region of the viral genome between M135 and M136 is replaced by a cassette expressing both eGFP and soluble TIM3 (FIG. 12A). Recombinant virus was then quadruple plaque purified on BSC40 cells by selecting GFP⁺ clones. Clonality of the final virus (vTIM3) was then confirmed using PCR.

In vitro Characterization of vTIM3: vTIM3 secretes soluble PD1 from infected cells. Secretion of soluble PD1 from virally infected cells was confirmed by western blotting supernatants from B16/F10 melanoma cells infected with either saline (mock), vGFP (control virus), or vTIM3 after 24 hours of infection. A strong band consistent with the soluble portion of TIM3 was observed specifically in the supernatant of cell infected with vTIM3 (FIG. 12D).

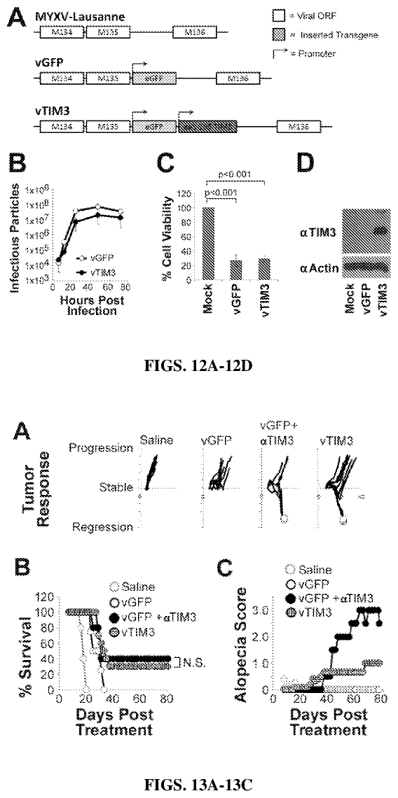

vTIM3 displays normal replication and oncolytic capacity in vitro. To determine whether insertion of the TIM3 transgene would alter MYXV replication, single step growth curves were performed on both vGFP and vTIM3 in a variety of cells. It was observed that both viruses displayed identical replication in all tested cell types (FIGS. 12B and 12C). To further test whether secretion of the TIM3 transgene would alter MYXV's ability to kill directly infected cells, it was next asked how effective both vGFP and vTIM3 were at killing B16/F10 melanoma cells. B16/F10 cells were infected with either vGFP or vTIM3 at the indicated multiplicities of infection. After 24 hours, cellular viability was analyzed using MTT assay. It was observed that both vGFP and vTIM3 displayed an identical capacity to kill infected melanoma cell in vitro (FIG. 13).

Oncolytic potential against melanoma in vivo. To test whether vTIM3 displayed increased oncolytic capacity in vivo, its ability to regress established melanoma tumors was tested in mice. C57/B6 mice were implanted subcutaneously with 5×10⁵ B16/F10 melanoma cells. Treatment was initiated seven days after injection of tumor cells (when tumors are approximately 15-20 mm²). Treatment consisted of two intratumoral injections of either saline, 1×10⁷ FFU of vGFP, or 1×10⁷ FFU of vTIM3 given on days 7 and 12. Animals were then monitored daily for tumor size and euthanized when tumors reached 150 mm in any direction Animals treated with vGFP displayed reduced tumor growth, however, the majority of tumors in these animals still progressed eventually requiring euthanasia (FIG. 13). In contrast, many mice treated with vTIM3 displayed rapid regression of established tumors resulting in complete durable remissions in 7/12 animals.

A series of viruses were constructed expressing variants of the soluble TIM3 protein in which previously validated binding sites for each TIM3 ligand have been removed through mutagenesis (FIG. 14). Each of these viruses are tested for their ability to induce a tumor immunity and eradicate established tumors in vivo. This identifies the mechanisms involved in vTIM3-based checkpoint blockade as well as by allowing for the construction of a next generation vTIM3 construct with improved therapeutic efficacy.

### Example 3—vMYX-Therapy in Metastatic Disease

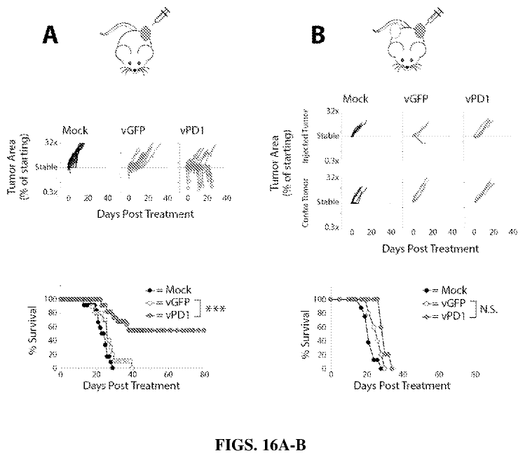

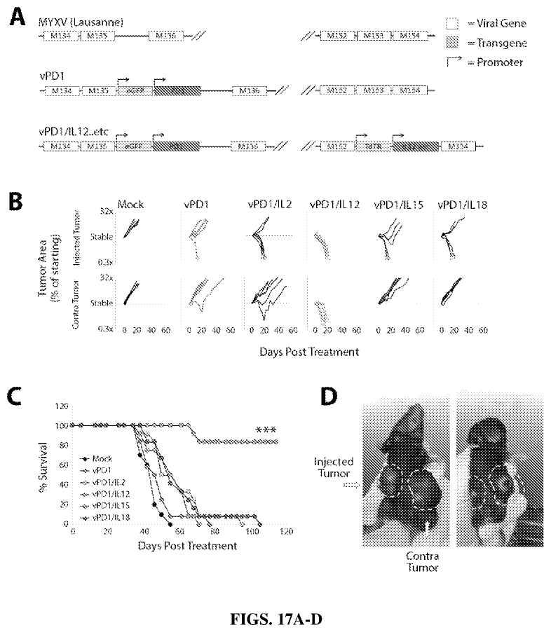

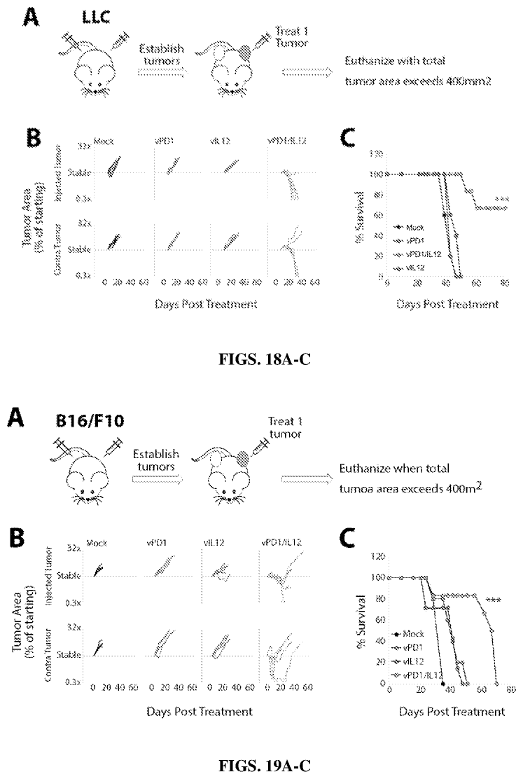

A recombinant MYXV which expresses a secreted form of soluble PD1 (vPD1) was also studied to determine its activity relative to metastatic cancers. Unfortunately, while vPD1 is extremely effective at eradicating localized disease (e.g., through the maintenance of anti-tumor immunity) see, e.g., FIG. 16, additional experiments have indicated that it has reduced effectiveness relative to non-injected, metastatic tumors (see FIG. 16B). Due to the inability of vPD1 to effectively regress non-treated lesions, to advance the clinical potential of this virus additional modifications were studied that could be added to the vPD1 backbone which would result in improved systemic efficacy. Additional recombinant viruses which expressed both soluble PD1 and either additional soluble T cell checkpoint proteins or a series of proinflammatory cytokines (see, FIG. 17A). Each of these viruses was then purified to clonality and tested for its ability to regress both injected and non-injected Lewis Lung Carcinomas (LLC) in a standard contralateral tumor model. The results indicated that, of the molecules tested, only inclusion of IL-12 (encoded as a p40/p35 fusion protein) significantly improved efficacy of the vPD1 backbone against non-injected lesions (FIG. 17B-C). Impressively, however, vPD1/IL12 was able to fully regress both injected and non-injected lesions in virtually all treated mice (durable complete response lasting >120 days in 10/12 animals) resulting in an effective ‘cure rate’ of almost 90%. Remarkably, even in animals with bulky, well-established disease (animals from the initial ‘mock’ cohort displaying a total tumor burden between 350-400 mm²), treatment of a single tumor with vPD1/IL12 could cause both complete elimination of the treated tumors as well as significant regression in the non-treated tumors (FIG. 17D). These studies demonstrate that vPD1/IL12 virus represented a novel therapeutic agent with strong clinical potential against even late stage, metastatic disease.

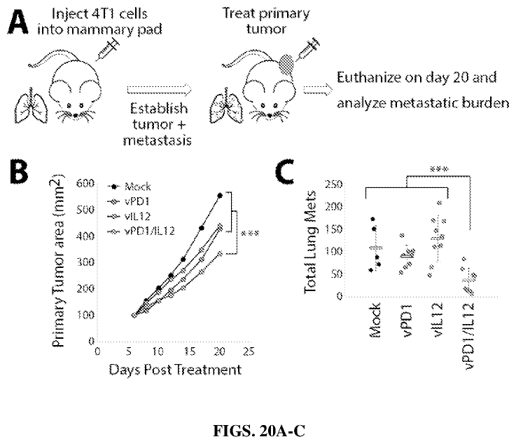

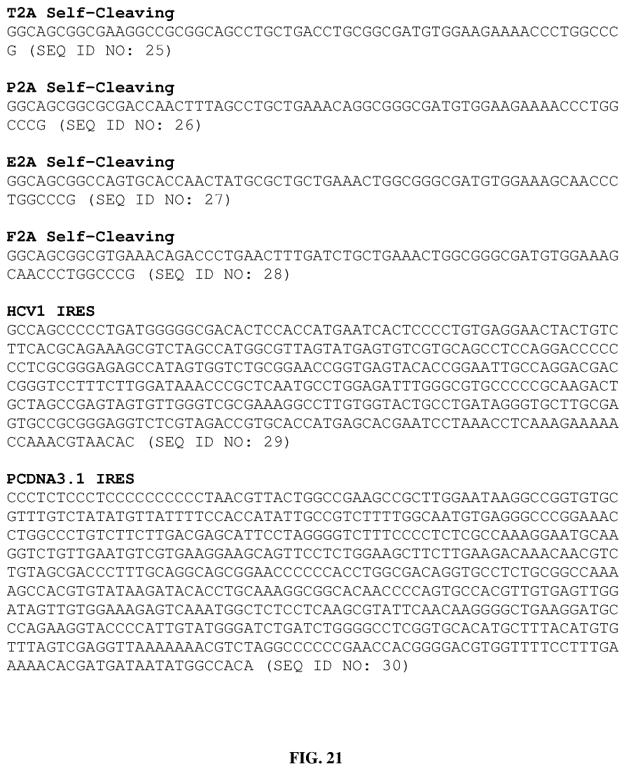

To advance on this exciting initial finding, the therapeutic breadth of the vPD1/IL12 virus was further studied. To accomplish this, a single recombinant MYXV expressing an IL12 fusion protein (vIL12) was compared the efficacy of this virus to that of both vPD1 and vPD1/IL12 in preclinical models of metastatic disease: LLC lung cancer (SQ contralateral tumor model), B16/F10 melanoma (SQ contralateral tumor model), 4T1 and triple negative breast cancer (single SQ tumor spontaneously metastatic to the lung). The results from all models clearly demonstrated that: 1) the vPD1/IL12 virus was capable of regressing both treated and non-treated lesions from a wide range of different malignancies including tumors representing both immunologically ‘hot’ (LLC and B16/F10) and immunologically ‘cold’ (4T1 and ID8) forms of disease. 2) This clinical efficacy was not observed in any model following treatment with either singly recombinant virus (vPD1 or vIL12) indicating that that vPD1/IL12's therapeutic effect is due to a unique form of combinatorial synergy (FIGS. 18-20).

### Example 4

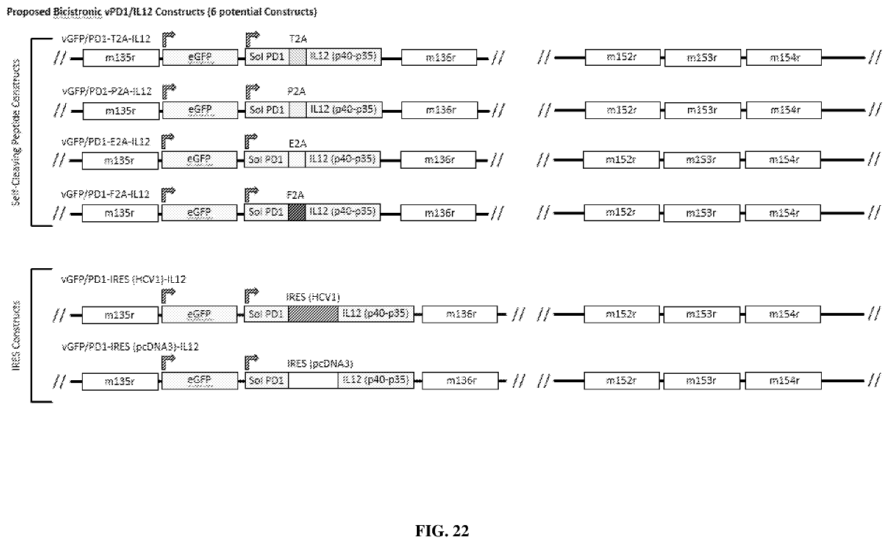

Plasmids encoding five proposed biscistronic constructs (vPBS-135-GFP-PD1-E2A-IL12-136, vPBS-135-GFP-PD1-F2A-IL12-136, vPBS-135-GFP-PD1-P2A-IL12-136, vPBS-135-GFP-PD1-HCV^{IRES}-IL12-136, vPBS-135-GFP-PD1-pcDNA3^{IRES}-IL12-136; FIG. 22) were commercially synthesized. Each plasmid was then transfected into BSC40 cells. 24 hours after transfection, cells were infected with myxoma virus (Strain Lausanne). 48 hours after infection, both the cells and supernatant were harvested. Samples were subsequently analyzed for expression of both the PD1 and IL12 transgenes via western blot.

Western blot analysis of the cellular fraction with anti-IL12 resulted in two discrete bands corresponding to the predicted molecular weight of the IL12 fusion protein and the uncleaved PD1/IL12 protein. The relative abundance of these two products predicts the efficacy of cleavage from the bicistronic plasmids utilizing self-cleaving peptides (E2A, F2A, and P2A) and suggests that P2A is cleaved more efficiently than the other two constructs. Western blot of the secreted fractions with anti-IL12 indicates that secretion of IL12 corresponds fairly well with production of the IL12 fusion protein and cleavage from the self-cleaving peptide constructs.

Western blot of the cellular fraction with anti-PD1 demonstrates strong staining for PD1 in the HCV and pcDNA constructs, indicating high expression of PD1 but possibly inefficient secretion. Possible uncleaved fusion protein is observed as a high molecular weight band in E2A and F2A. Western blot of the secreted fractions with anti-PD1 indicates high level section from the P2A, HCV, and pcDNA constructs.

The E2A and F2A constructs produce high levels of the fusion protein, but appear to have relatively inefficient cleavage from the self-cleaving peptides resulting in poor transgene secretion. The fact that HCV expresses PD1, but not IL12 suggests the IRES in this construct is not functioning. Both P2A and pcDNA constructs appear to be functioning well.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. References

The following references, to the extent that they provide exemplary procedural or other details supplementary to those set forth herein, are specifically incorporated herein by reference.
Ausubel et al., Current Protocols in Molecular Biology, Greene Publ. Assoc. Inc. & John Wiley & Sons, Inc., MA, 1996.
Bartee et al., 2017. Cancer Research 77, 2952-2963.
Cameron et al., Virology, 264:298-318, 1999.
Chen and Okayama, Mol. Cell Biol., 7(8):2745-2752, 1987.
Cheng et al. “Membrane-Tethered Proteins for Basic Research, Imaging, and Therapy” Medicinal Research Reviews, Vol. 28, No. 6, 885-928, 2008.
Fechheimer et al., Proc Natl. Acad. Sci. USA, 84:8463-8467, 1987.
Fraley et al., Proc. Natl. Acad. Sci. USA, 76:3348-3352, 1979.
Frankel and Pabo, Cell, 55(6):1189-1193, 1988.
Gopal, Mol. Cell Biol., 5:1188-1190, 1985.
Graham and Van Der Eb, Virology, 52:456-467, 1973.
Harland and Weintraub, J. Cell Biol., 101(3):1094-1099, 1985.
Johnson and Win, 2017. Oncoimmunology, Combination therapy with PD-1/PD-L1 blockade: An overview of ongoing clinical trials. DOI:10.1080/2162402X.2017.1408744
Johnston and McFadden, J. Virology, 77:6093-6100, 2003.
Kaeppler et al., Plant Cell Rep., 8:415-418, 1990.
Kaneda et al., Science, 243:375-378, 1989.
Kato et al, J. Biol. Chem., 266:3361-3364, 1991.
Kaufman, et al., 2015. Nature Rev. Drug Discov 14, 642-662.
Kerr and McFadden, Viral Immunology, 15:229-246, 2002.
Levin et al. (2012) Nature 484:529-533
Liu et al., J. Clin. Investig., 105:1613-1621, 2000.
Lucas and McFadden, J. Immunol., 173:4765-47742004.
Na et al., 2016. Cell Res 27:147-150.
Nabel et al., Science, 244(4910):1342-1344, 1989.
Nicolau and Sene, Biochim. Biophys. Acta, 721:185-190, 1982.
Nicolau et al., Methods Enzymol., 149:157-176, 1987.
Pardoll, 2012. Nature Reviews: Cancer 12:252-264.
PCT Application No. WO 04/078206.
PCT Application No. WO 94/09699.
PCT Application No. WO 95/06128.
Potrykus et al., Mol. Gen. Genet., 199(2):169-177, 1985.
Potter et al., Proc. Natl. Acad. Sci. USA, 81:7161-7165, 1984.
Rippe, et al., Mol. Cell Biol., 10:689-695, 1990.
Sambrook and Russell, Molecular Cloning: A Laboratory Manual, 3^{rd} Ed. Cold Spring Harbor Lab. Press, 2001.
Sambrook et al., In: Molecular Cloning: A Laboratory Manual, Vol. 1, Cold Spring Harbor Laboratory Press, Cold Spring Harbor, N.Y., (7)7:19-17.29, 1989.
Smallwood, S. E. et al., Curr Protoc Microbiol., Unit-14A.1, 2010.
Sypula et al., Gene Ther Mol Biol., 8:103-1142004.
Tan, et al., 2017. Nature Communications 8:14369-10.
Topalian, S. L., et al., 2015. Cancer Cell 27:450-461.
Tosic et al., PLoS One, e109801, 2014.
Tur-Kaspa et al., Mol. Cell Biol., 6:716-718, 1986.
U.S. Patent Application No. 20090035276.
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,945,100
U.S. Pat. No. 5,981,274
U.S. Pat. No. 5,994,624
U.S. Pat. No. 8,613,915
Wilson et al., Science, 244:1344-1346, 1989.
Wong et al., Gene, 10:87-94, 1980.
Wu and Wu, Biochemistry, 27: 887-892, 1988.
Wu and Wu, J. Biol. Chem., 262:4429-4432, 1987.

1. A recombinant oncolytic myxoma virus comprising expression cassettes encoding:
(a) a soluble form of programmed cell death protein 1 (PD1);
(b) interleukin 12 (IL-12); and
(c) a tumor antigen or a cytokine/chemokine other than interleukin 12 (IL-12), wherein the myxoma virus is replication competent and wherein two of said expression cassettes are provided a dicistronic expression cassette.

2. The oncolytic myxoma virus of claim 1, wherein the virus comprises a cytokine/chemokine selected from the group consisting of IL-2, IL-4, IL-15, IL-17, IL-18 (mutated), IL-23, IL-35, IL-36, IFN-γ, IFN-β, RANTES/CCL5, GM-CSF, cGAS, or Ebola GP (aa1-298).

3. The oncolytic myxoma virus of claim 1, wherein the virus comprises a tumor antigen selected from the group consisting of p53, MUC1, PSMA, mRAS or S100P.

4. A recombinant myxoma oncolytic virus comprising one or more expression cassettes encoding a (a) mutant soluble form of PD1 (mutPD1), (b) interleukin 12 (IL-12), and (c) a cytokine/chemokine other than interleukin 12 (IL-12) or a tumor antigen, wherein the myxoma virus is replication competent, and wherein the mutPD1 prevents recognition of mutPD1 by an anti-PD1 antibody.

5. The oncolytic myxoma virus of claim 4, wherein the virus comprises a cytokine/chemokine selected from the group consisting of IL-2, IL-4, IL-15, IL-17, IL-18 (mutated), IL-23, IL-35, IL-36, IFN-γ, IFN-β, RANTES/CCL5, GM-CSF, cGAS, or Ebola GP (aa1-298).

6. The oncolytic myxoma virus of claim 4, wherein the virus comprises a tumor antigen selected from the group consisting of p53, MUC1, PSMA, mRAS or S100P.

7. The oncolytic myxoma virus of claim 4, wherein the mutPD1 contains a mutation in the CD loop that prevents antibody recognition by anti-PD1 antibodies.

8. The oncolytic myxoma virus of claim 7, wherein the mutPD1 contains a point mutation in the CD loop comprising D85G.

9. The oncolytic myxoma virus of claim 8, wherein the mutPD1 is not recognized by pembrolizumab.

10. The oncolytic myxoma virus of claim 1, wherein the soluble PD1/mutant soluble PD1 comprises an extracellular region of human PD1.

11. The oncolytic myxoma virus of claim 1, wherein the soluble PD1/mutant soluble PD1 and the IL-12 are encoded in the dicistronic expression cassette.

12. The oncolytic myxoma virus of claim 1, wherein the soluble PD1/mutant soluble PD1 and the IL-12 are encoded in distinct expression cassettes.

13. The oncolytic myxoma virus of claim 1, wherein the soluble PD1/mutant soluble PD1 and the chemokine/cytokine are encoded in the dicistronic expression cassette.

14. The oncolytic myxoma virus of claim 1, wherein the soluble PD1/mutant soluble PD1 and the chemokine/cytokine are encoded in distinct expression cassettes.

15. The oncolytic myxoma virus of claim 1, wherein the soluble PD1/mutant soluble PD1 and the tumor antigen are encoded in the dicistronic expression cassette.

16. The oncolytic myxoma virus of claim 1, wherein the soluble PD1/mutant soluble PD1 and the tumor antigen are encoded in distinct expression cassettes.

17. The oncolytic myxoma virus of claim 1, wherein the IL-12 and the tumor antigen are encoded in the dicistronic expression cassette.

18. The oncolytic myxoma virus of claim 1, wherein the IL-12 and the tumor antigen are encoded in distinct expression cassettes.

19. The oncolytic myxoma virus of claim 12, wherein a first expression cassette is inserted in the intergenic region between the m135r and m136r ORFs and a second expression cassette is inserted between the m152r and m154r ORFs and replaces the mR153r ORF.

20. The oncolytic myxoma virus of claim 14, wherein a first expression cassette is inserted in the intergenic region between the m135r and m136r ORFs and a second expression cassette is inserted between the m152r and m154r ORFs and replaces the mR153r ORF.

21. The oncolytic myxoma virus of claim 16, wherein a first expression cassette is inserted in the intergenic region between the m135r and m136r ORFs and a second expression cassette is inserted between the m152r and m154r ORFs and replaces the mR153r ORF.

22. The oncolytic myxoma virus of claim 18, wherein a first expression cassette is inserted in the intergenic region between the m135r and m136r ORFs and a second expression cassette is inserted between the m152r and m154r ORFs and replaces the mR153r ORF.

23. The oncolytic myxoma virus of claim 1, wherein the dicistronic expression cassette comprises an internal ribosome entry site (IRES) between the coding sequences of the expression cassettes.

24. The oncolytic myxoma virus of claim 23, wherein the IRES is a cellular IRES.

25. The oncolytic myxoma virus of claim 24, wherein the IRES is an IRES from eIF4G, BCL2, BiP, or c-IAP1.

26. The oncolytic myxoma virus of claim 23, wherein the IRES is a viral IRES.

27. The oncolytic myxoma virus of claim 26, wherein the IRES is an IRES from poliovirus (PV), encephalomyelocarditis virus (EMCV), classical swine-fever virus (CSFV), foot-and-mouth disease virus (FMDV), human immunodeficiency virus (HIV), bovine viral diarrhea virus (BVDV), hepatitis C virus (HCV) or cricket paralysis virus (CrPV).

28. The oncolytic myxoma virus of claim 27, wherein the IRES is an IRES from HCV.

29. The oncolytic myxoma virus of claim 1, wherein the dicistronic expression cassette comprises a polyprotein of the coding sequences of the expression cassettes.

30. The oncolytic myxoma virus of claim 29, wherein the polyprotein comprises a protease cleavage site between the proteins encoded by the two expression cassettes.

31. The oncolytic myxoma virus of claim 30, wherein the protease cleavage site is cleaved by cellular protease.

32. The oncolytic myxoma virus of claim 30, wherein the protease cleavage site is a self-cleaving peptide.

33. The oncolytic myxoma virus of claim 32, wherein the self-cleaving peptide is a viral self-cleaving peptide.

34. The oncolytic myxoma virus of claim 33, wherein the self-cleaving peptide is a T2A, P2A, E2A or F2A peptide.

35. The oncolytic myxoma virus of claim 1, wherein the expression cassette(s) is/are under the control of one or more viral promoters.

36. The oncolytic myxoma virus of claim 35, wherein the one or more viral promoters is/are synthetic early/late poxvirus promoter.

37. The oncolytic myxoma virus of claim 35, wherein the synthetic early/late poxvirus promoter is at least 90% identical to AAAATTGAAATTTTATTTTTTTTTTTTGGAATATAAATA (SEQ ID NO: 14).

38. The oncolytic myxoma virus of claim 15, further comprising a marker gene.

39. The oncolytic myxoma virus of claim 1, wherein IL-12 is fused to a transmembrane domain.

40. The oncolytic myxoma virus of claim 39, wherein the transmembrane domain is encoded by SEQ ID NO: 12.

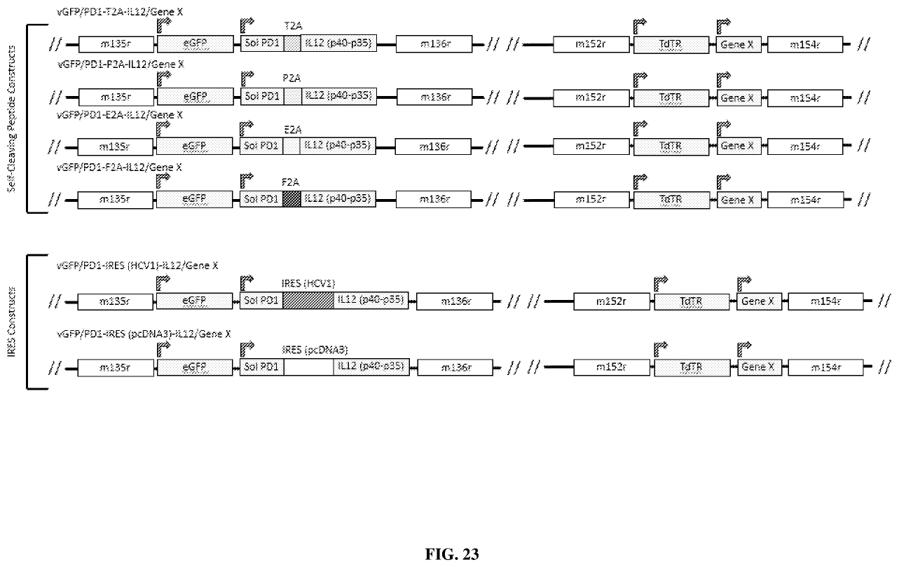

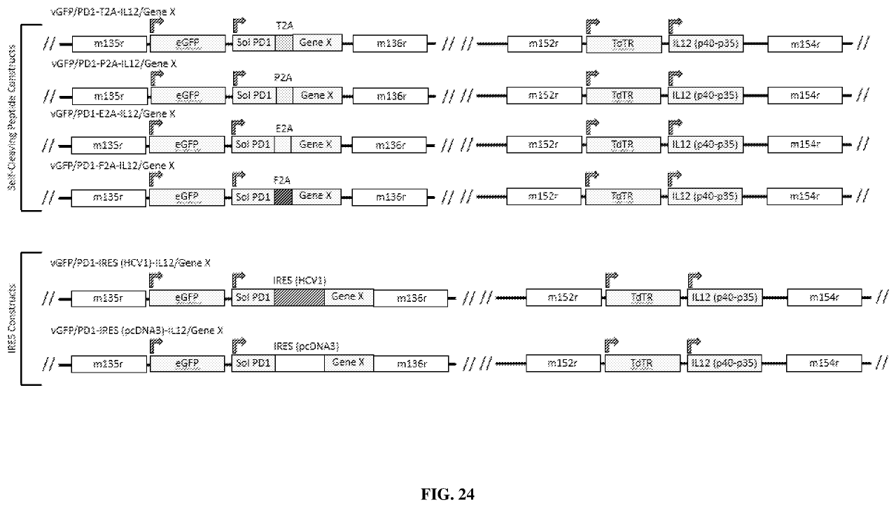

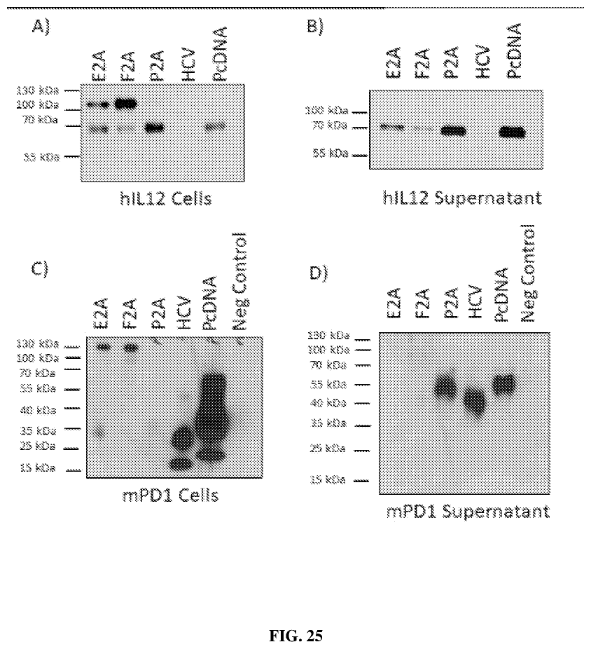

41. The oncolytic myxoma virus of claim 1, wherein the oncolytic myxoma virus comprises a construct according to one of FIGS. 22-24.

42. A pharmaceutical composition of the oncolytic myxoma virus of claim 1.

43. A method of treating a disease in a subject in need thereof comprising administering an effective amount of the oncolytic myxoma virus of claim 1.

44. The method of claim 43, wherein the disease is cancer.

45. The method of claim 44, wherein the cancer has increased expression of programmed death-ligand 1 (PDL1).

46. The method of claim 44, wherein the subject has been determined to have a cancer that expresses increased PDL1.

47. The method of claim 44, wherein the cancer does not have increased expression of PDL1.

48. The method of claim 44, wherein the cancer is melanoma, kidney cancer, colorectal cancer, breast cancer, lung cancer, head and neck cancer, brain cancer, leukemia, prostate cancer, bladder cancer, and ovarian cancer.

49. The method of claim 44, wherein the cancer is melanoma.

50. The method of claim 49, wherein the melanoma is metastatic melanoma.

51. The method of claim 43, wherein the oncolytic myxoma virus is administered intra-arterially, intravenously, intraperitoneally, or intratumorally.

52. The method of claim 43, wherein the oncolytic myxoma virus is administered two or more times.

53. The method of claim 43, further comprising administering at least a second anti-cancer therapy to the subject.

54. The method of claim 53, wherein the second anti-cancer therapy is administered concurrently or sequentially with the recombinant virus.

55. The method of claim 53, wherein the second anti-cancer therapy is an immunomodulator.

56. The method of claim 53, wherein the second anti-cancer therapy is immunotherapy, chemotherapy, radiotherapy, gene therapy, surgery, hormonal therapy, anti-angiogenic therapy or cytokine therapy.

57. The method of claim 56, wherein the immunotherapy is immune checkpoint inhibitor therapy.

58. The method of claim 57, wherein the immune checkpoint inhibitor therapy comprises treatment with an antibody directed to PD1, PDL1, or CTLA4.

59. The method of claim 58, wherein the antibody is Pembrolizumab, Nivolumab, Atezolizumab, Avelumab, Durvalumab, or Ipilimumab.

60. The method of claim 59, wherein the antibody is Pembrolizumab.

61. A method of treating a disease in a subject in need thereof comprising:
(a) testing the subject for overexpression of PDL1; and
(b) administering to a subject with increased expression of PDL1 a therapeutically effective amount of the oncolytic myxoma virus of claim 1.

62. The method of claim 61, wherein the disease is cancer.

63. The method of claim 62, wherein the cancer has increased expression of programmed death-ligand 1 (PDL1).

64. The method of claim 62, wherein the cancer does not have increased expression of PDL1.

65. The method of claim 62, wherein the cancer is melanoma, kidney cancer, colorectal cancer, breast cancer, lung cancer, head and neck cancer, brain cancer, leukemia, prostate cancer, bladder cancer, and ovarian cancer.

66. The method of claim 62, wherein the cancer is melanoma.

67. The method of claim 64, wherein the melanoma is metastatic melanoma.

68. The method of claim 61, wherein the oncolytic myxoma virus is administered intra-arterially, intravenously, intraperitoneally, or intratumorally.

69. The method of claim 61, wherein the oncolytic myxoma virus is administered two or more times.

70. The method of claim 61, further comprising administering at least a second anti-cancer therapy to the subject.

71. The method of claim 70, wherein the second anti-cancer therapy is administered concurrently or sequentially with the oncolytic myxoma virus.

72. The method of claim 70, wherein the second anti-cancer therapy is an immunomodulator.

73. The method of claim 70, wherein the second anti-cancer therapy is chemotherapy, immunotherapy, radiotherapy, gene therapy, surgery, hormonal therapy, anti-angiogenic therapy or cytokine therapy.

74. The method of claim 73, wherein the immunotherapy is immune checkpoint inhibitor therapy.

75. The method of claim 74, wherein the immune checkpoint inhibitor therapy comprises treatment with an antibody directed to PD1, PDL1, or CTLA4.

76. The method of claim 75, wherein the antibody is Pembrolizumab, Nivolumab, Atezolizumab, Avelumab, Durvalumab, or Ipilimumab.


SEQUENCE LISTING (30 sequences)

SEQ ID NO: 1 - DNA, 73 bp, Artificial sequence
  Synthetic primer
atcgcccgggaaaaattgaaattttattttttttttttggaatataaataaccatgcagatcccacaggcgcc

SEQ ID NO: 2 - DNA, 33 bp, Artificial sequence
  Synthetic primer
atcggaattctcaggtttggaactggccggctg

SEQ ID NO: 3 - DNA, 504 bp, Homo sapiens
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaacc

SEQ ID NO: 4 - protein, 168 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQT

SEQ ID NO: 5 - protein, 168 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEGRSQPGQDCRFRVTQLPNGRDFHMSVVRARRNDSGTYLCGAISLAPKAQIKESLRAELRVTERRAEVPTAHPSPSPRPAGQFQT

SEQ ID NO: 6 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 8 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - protein, 164 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLTAAAHHHHHHHH

SEQ ID NO: 10 - DNA, 3042 bp, Artificial sequence
  Synthetic construct
  misc_feature (1)..(10): n is a, c, g, or t
  misc_feature (12)..(12): n is a, c, g, or t
  misc_feature (15)..(15): n is a, c, g, or t
  misc_feature (18)..(18): n is a, c, g, or t
  misc_feature (622)..(624): n is a, c, g, or t
  misc_feature (1391)..(1391): n is a, c, g, or t
  misc_feature (1860)..(1860): n is a, c, g, or t
nnnnnnnnnncncgnggnggcggccgctctagtaggattacctggtctatatagataacaaaacctacgtacgtataaacgagaccgttgtaccggagaacgagtatctggcagcgaaggccccgcgagtgacctgtttccacacggacttgatccccattacggacgaagagacacaacgacgttttgagaaaatgattgtacaggcggcgttagaggacgccctaacgagcatctttgaggagcacgacaataacgtaaccgattacttcgcggaatacatgcgatccctccaaatggcgaataaaagtcatacgaataatattatcgcggtcgctttagcggggataatcgtcattgtaacgacctacgtgtttactagattacgcactaagcaaaaaaaaggaaattataacgtacgtaataagatagataattccatacagaaagagattcagttggacggtgtatatactactgacaacgtttttatataaacatggtgtttatatttattatcacctgtgtatgtttggtgacgagatcctgtgggggtgggttagaagacgatatagatcgcatatttcaaaaacgatacaacgaactgagccagccgattaannncaatatgcgtacactgtgcaagtttagaggaattaccgcgactatgtttacggaaggagaatcttaccttattcaatgtcccataattcacgattacgtgctacgggcgctgtatgacttagtggaaggaagttacacggtacgctgggaacgcgaaacggaagacgatgttgagtcggtagatccgaagttagtcaaagggacgctattatacctccaacctaacgcgtccagtataggaacgtatctatgtaccttacacgataaccgaggtatgtgttatcaatctgtcgcgcacgtcatccgacgtccgaagatgcaatgcgtgaaacatgcacatacgacatcggacagcaacctgtggatatacctcgccattttagcagttttgatatccttaggcgtcctgtaaaggaaacgcgccagactccggaactatgaaggatttatcactgtatacagactccgacgtacgaaggataatcacgacgtaactcgaactctgcaggtcgactctagaggatctactagtcatatggatttaaaaatagcggagcttaaaaattgaaattttattttttttttttggaatataaataagctcgaagtcgacagatctaggcctggtacccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacntacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgntgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgggtaattacccgggatgttttcaggtcttaccctcaactgtgtcctgctgctgctgcaactactacttgcaaggtcattggaaaatgcttatgtgtttgaggttggtaagaatgcctatctgccctgcagttacactctatctacacctggggcacttgtgcctatgtgctggggcaagggattctgtccttggtcacagtgtaccaacgagttgctcagaactgatgaaagaaatgtgacatatcagaaatccagcagataccagctaaagggcgatctcaacaaaggagacgtgtctctgatcataaagaatgtgactctggatgaccatgggacctactgctgcaggatacagttccctggtcttatgaatgataaaaaattagaactgaaattagacatcaaagcagccaaggtcactccagctcagactgcccatggggactctactacagcttctccaagaaccctaaccacggagagaaatggttcagagacacagacactggtgaccctccataataacaatggaacaaaaatttccacatgggctgatgaaattaaggactctggagaaacgatcagaactgctatccactaggaattctaacattttttaaaacaatttcgttatgttaaattatggaacggtcgcccacttacacggtacacgataaacgcttttctatcgtcgcactaaacggacaatacgacatggtggacgattttggtcttagtttttcttacacagcgatcgacgatatttctaaaaatcattccatcaaacacgttttagaagaatacttttcatggcgcgcgtatataggccgggtatgtatcataccgaatcacgtgggaaagctctacatcaaacttacaaagttggacaccacggcgaagaacaaactaggcaatctagatatattgttatgcgacgtgttaaaaatagacgaggacggaggcaacgagaaactgtttcaattcatacggtcgcggatcccccaattcgatatcaagcttatcgataccgtcgacctcg

SEQ ID NO: 11 - DNA, 585 bp, Artificial sequence
  Synthetic construct
atgttttcaggtcttaccctcaactgtgtcctgctgctgctgcaactactacttgcaaggtcattggaaaatgcttatgtgtttgaggttggtaagaatgcctatctgccctgcagttacactctatctacacctggggcacttgtgcctatgtgctggggcaagggattctgtccttggtcacagtgtaccaacgagttgctcagaactgatgaaagaaatgtgacatatcagaaatccagcagataccagctaaagggcgatctcaacaaaggagacgtgtctctgatcataaagaatgtgactctggatgaccatgggacctactgctgcaggatacagttccctggtcttatgaatgataaaaaattagaactgaaattagacatcaaagcagccaaggtcactccagctcagactgcccatggggactctactacagcttctccaagaaccctaaccacggagagaaatggttcagagacacagacactggtgaccctccataataacaatggaacaaaaatttccacatgggctgatgaaattaaggactctggagaaacgatcagaactgctatccac

SEQ ID NO: 12 - DNA, 177 bp, Artificial sequence
  Synthetic construct
cttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctt

SEQ ID NO: 13 - DNA, 7620 bp, Artificial sequence
  Synthetic construct
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgcataaacgcgtttaaacagtcccccgtacgcggtacatcgtacgcacacttcactaacgatgtcgtacatcgattacacaaagaagtagagtcatacgacgtacgtttccctataaaatcggtaaacctagacgcggtgtttctatccataaacgtaacacgtgtacgtctacgttggaagatacccttgaccgaacacaatccttatcagacggcctacggatgttctaacgacagattatacagctacaacgagtacgctttttctcatttaaaacaagaccgtgtaaagatcatagaactcccatgtgacgacgattacagcgtcgtgttaatcacacacgatagccgttcgactattacaccggataaagtgaccgggtggctgcgcacgacccgtctacgttacgtaaacgtatccctacccaagggttccacggaaacgggacacaacgtaacgtgtctaactcccacacacgtcaatctatgtcatcgttgtcgtataacgattaccaaaacgggcgtggacgcaaccgcgttctcatgcgtcgacggcgatacatgcaccgaacacgacacgaccgcgtcaacgtgtacgattattataaaaacgacgggactggactttttgtttatggggaaactctaaaaaaaattgtcaattaaagtaactgcagatcgatcgcatatgaaaattgaaattttattttttttttttggaatataaataatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctccaagacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctccaagacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaacccgggaaaaattgaaattttattttttttttttggaatataaataaccatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcccttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttgagaattcacgaatcgaataaaaacccgtgtacacacggacgttaattttttttgtggtttaaaaaatgaccacatttacgcttttttttaacgcgttatataaggtatctcgtttgtctataacaaagatcgtaactgaccttttttatatcgagaaaacatacgtttagttcatcctcaaacgtaacaccgtaactgcctcggacatcctccttgttgtcgtacacaaacatactaatcggatgcgtgaaatgaggattcactttaatcggattggtttctaggttaacacatgttacacaagatcctaagatggttatggacacatccttgttgtgatgtaacgagtcgggaagttgattgccgtagttgcccacgtcgccctccggttccagacacgtaatggttaggtatatatccgaatacttcgtcaacggatgagtcgtaaataacatgatggatagcttgttcccatctcctgcaccagcactggccgccacaaatcgttgtaccacgttagtaatcgtaatgtttatcataagcccgtacccggttaatatgagcgtggacgttttatgatcgtatcgttccttcatgtgacattctcccataaccgtttcgacgtaccgatttaacccgatggttagctcggcggctaagtgccagtggatcccccaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 14 - DNA, 39 bp, Artificial sequence
  Synthetic promoter
aaaattgaaattttattttttttttttggaatataaata

SEQ ID NO: 15 - protein, 21 aa, Artificial sequence
  Synthetic peptide
  MISC_FEATURE (1)..(3): May or may not be present
GSGEGRGSLLTCGDVEENPGP

SEQ ID NO: 16 - protein, 22 aa, Artificial sequence
  Synthetic peptide
  MISC_FEATURE (1)..(3): May or may not be present
GSGATNFSLLKQAGDVEENPGP

SEQ ID NO: 17 - protein, 23 aa, Artificial sequence
  Synthetic peptide
  MISC_FEATURE (1)..(3): May or may not be present
GSGQCTNYALLKLAGDVESNPGP

SEQ ID NO: 18 - protein, 25 aa, Artificial sequence
  Synthetic peptide
  MISC_FEATURE (1)..(3): May or may not be present
GSGVKQTLNFDLLKLAGDVESNPGP

SEQ ID NO: 19 - protein, 26 aa, Artificial sequence
  Synthetic peptide
NQTDKLAAFPEDRSQPGQDCRFRVTQ

SEQ ID NO: 20 - protein, 26 aa, Artificial sequence
  Synthetic peptide
NQTEKQAAFCNGLSQPVQDARFQIIQ

SEQ ID NO: 21 - protein, 26 aa, Artificial sequence
  Synthetic peptide
NQTEKQAAFCNGYSQPVRDARFQIVQ

SEQ ID NO: 22 - protein, 26 aa, Artificial sequence
  Synthetic peptide
NQTDKLAAFQEDRIEPGRDRRFRVMR

SEQ ID NO: 23 - protein, 26 aa, Artificial sequence
  Synthetic peptide
NQTDKLAAFREDSSQPGRSGRFRVTR

SEQ ID NO: 24 - protein, 194 aa, Artificial sequence
  Synthetic polypeptide
MFSGLTLNCVLLLLQLLLARSLEDGYKVEVGKNAYLPCSYTLPTSGTLVPMCWGKGRCPWSQCTNELLRTDERNVTYQKSSRYQLKGDLNKGDVSLIIKNVTLDDHGTYCCRIQFPGLMNDKKLELKLDIKAAKVTAQTAHGDSTTASPRTLTTERNGSETQTLVTLHNNNGTKISTWADEIKDSGETIRTAIH

SEQ ID NO: 25 - DNA, 63 bp, Artificial sequence
  Synthetic nucleic acid
ggcagcggcgaaggccgcggcagcctgctgacctgcggcgatgtggaagaaaaccctggcccg

SEQ ID NO: 26 - DNA, 66 bp, Artificial sequence
  Synthetic nucleic acid
ggcagcggcgcgaccaactttagcctgctgaaacaggcgggcgatgtggaagaaaaccctggcccg

SEQ ID NO: 27 - DNA, 69 bp, Artificial sequence
  Synthetic nucleic acid
ggcagcggccagtgcaccaactatgcgctgctgaaactggcgggcgatgtggaaagcaaccctggcccg

SEQ ID NO: 28 - DNA, 75 bp, Artificial sequence
  Synthetic nucleic acid
ggcagcggcgtgaaacagaccctgaactttgatctgctgaaactggcgggcgatgtggaaagcaaccctggcccg

SEQ ID NO: 29 - DNA, 385 bp, Artificial sequence
  Synthetic nucleic acid
gccagccccctgatgggggcgacactccaccatgaatcactcccctgtgaggaactactgtcttcacgcagaaagcgtctagccatggcgttagtatgagtgtcgtgcagcctccaggaccccccctcgcgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccgcgggaggtctcgtagaccgtgcaccatgagcacgaatcctaaacctcaaagaaaaaccaaacgtaacac

SEQ ID NO: 30 - DNA, 583 bp, Artificial sequence
  Synthetic nucleic acid
ccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca